(19) United States
(12) Patent Application Publication
(10) Pub. No.: US 2014/0193449 A1
(43) Pub. Date: Jul. 10, 2014

(54) COMPOSITIONS AND METHODS FOR CANCER TREATMENT

(71) Applicant: University Health Network, Toronto (CA)

(72) Inventors: Jeffrey A. Medin, North York (CA); Andrea McCart, Toronto (CA); Severine Loisel-Meyer, Montpellier (FR)

(73) Assignee: University Health Network, Toronto (CA)

(21) Appl. No.: 14/149,911

(22) Filed: Jan. 8, 2014

Related U.S. Application Data
(63) Continuation of application No. 12/598,874, filed on Oct. 11, 2010, now abandoned.
(60) Provisional application No. 60/916,136, filed on May 4, 2007.

(51) Int. Cl. A61K 39/21; A61K 39/00

(52) U.S. Cl. CPC A61K 39/21; A61K 39/0011
USPC 424/187.1; 424/277.1; 435/320.1; 435/235.1; 435/375

(57) ABSTRACT

Compositions and methods for delivering tumor associated antigens and immune modulatory molecules to result in a therapeutic effect are disclosed. The compositions and methods use stably integrating lentiviral delivery systems. The methods are useful for therapeutically and prophylactically treating cancer such as colon cancer.

### CROSS-REFERENCE TO RELATED APPLICATIONS

This application is a continuation of U.S. patent application Ser. No. 12/598,874, which is a National Stage application based on International Application No. PCT/CA2008/000848, filed May 5, 2008, which claims priority to U.S. Patent Application No. 60/916,136, filed May 4, 2007, the disclosures of which are incorporated herein by reference.

### INCORPORATION OF SEQUENCE LISTING

A computer readable form of the Sequence Listing “10723-P4537US03_SequenceListing.txt” (73,008 bytes), submitted via EFS-WEB and amended on Jan. 7, 2013, is herein incorporated by reference.

### FIELD

The disclosure relates generally to compositions and methods for therapeutically and prophylactically treating cancer. In particular, the present disclosure pertains to lentiviral vectors for transducing cells with immunotherapeutic molecules and use of the transduced cells for cancer immunotherapy.

### BACKGROUND

Cancer immunotherapy aims to overcome the inability of the immune system to efficiently protect against the establishment of tumors or reject established tumors. Dendritic cells (DCs) are potent antigen-presenting cells that have been widely used to initiate or enhance tumor-associated antigen (TAA)-specific immune responses in animal models and clinical settings. Numerous reports show that modifying DCs via TAA peptide- or tumor lysate-pulsing can induce anti-tumor immunity [1-5]. Transfecting DCs with nucleic acid sequences encoding TAAs carries the advantage of inducing immunity towards a larger repertoire of naturally-derived MHC class I and II compatible peptides. Comparative studies have shown that by transfecting DCs with RNA, stronger anti-tumor effects can be achieved than by pulsing DCs with peptides [6-9].

Viral transduction of DCs offers similar advantages as RNA transfection, with the added potential benefits of more efficient transgene delivery and stable transgene expression, depending on the choice of virus. Retroviruses, including oncoretroviruses and lentiviruses, can also be used to effectively transduce DCs with one or more genes. Their integration into the host genome provides a way to generate long-term stable transgene expression. In recent reports, lentiviruses have been used to efficiently transduce murine and human DCs with TAAs [10-14]. Lentiviruses are well-suited for transducing DCs because they are capable of efficiently transducing slowly-dividing cells.

### Cancer

Colorectal cancer is the 2^{nd} leading cause of cancer death in Canada and the US. There are minimal treatment options and metastatic disease is often incurable. Two thirds of patients will develop recurrent or metastatic disease. Development of effective immunotherapy should prevent relapses and reduce the burden of metastatic disease in colon cancer and other metastatic cancers.

### Dendritic Cells (DCs)

DCs are the most potent antigen presenting cells (APCs). Derived from primitive CD34⁺ hematopoietic cells, they elicit B and T cell immune responses. By presenting tumor associated antigens, dendritic cells can elicit powerful immune responses directed towards tumors expressing the presented tumor associated antigen. DCs express MHC and accessory co-stimulatory/adhesive molecules. A major advantage in using DCs for the development of clinical gene therapies is that no DC-based leukemias have been reported.

### Vaccination Strategies

Vaccination strategies have been unsuccessfully attempted for the tumor associated antigen (TAA) carcinoembryonic antigen or CEA. CEA has been recognized as an optimal TAA. The most popular strategies have delivered CEA as a plasmid vaccine (1), a recombinant protein (2,3), or expressed it from poxviruses (4-6). Trials of peptide-loaded DCs have also been done (7-9). Using adenoviruses (Ad) expressing the entire CEA protein to infect DCs attempted to overcome some of the limitations of other strategies(10,11). However, many patients have pre-existing immunity to the Ad strains used. Phase I clinical trials of various vaccination strategies have been attempted (12-15) but in general the clinical results have been unsatisfactory.

### Lentiviral Vectors (LVs)

LVs are efficient gene transfer agents. They are stable and can be concentrated by ultracentrifugation to high titers. Compared to Ad, for example, they generate little immune consequences on their own reducing responses against transduced DCs. Articles that have used LV in relation to treatment of cancer cells include: the ex vivo transduction of DCs with melanoma TAAs (Metharom, P. et al., 2001; Firat, H. et al., 2002), the induction of DCs (Esslinger, C. et al., 2003) and antigen presentation for CTL responses (Breckpot, K. et aL, 2003; Esslinger, C. et al., 2003), and the transduction of CD34+ cells differentiated into DCs towards HIV/AIDS immunotherapy DCs (Gruber, A. et al., 2003). There remains a need for a useful LV cancer vaccination strategy.

### Interleukin-12

Immunotherapies are being developed to provide novel approaches to treat cancer, either alone or in addition to conventional treatments. These treatments are based on the notion that cancer cells express antigens that can be targeted by immune mechanisms and recognized by the acquired immune system. However, despite the presence of such antigens, tumors are generally not readily recognized and eliminated by the host, as evidenced by the development of disease. The inability of the immune system to protect against tumors may be due to mechanisms of evasion, active suppression, or sub-optimal activation of the response.

Cytokines are integral to both the innate and acquired immune systems. They can alter the balance of cellular and humoral responses, alter class switching of B lymphocytes and modify innate responses. These traits have made a number of cytokines interesting candidates for cancer innnnunotherapies.¹⁻³ Among these, IL-12 has been tested for its ability to promote immune recognition and response against tumors.

Interleukin-12 is a heterodimeric cytokine with multiple biological effects on the immune system. It is composed of two subunits, p35 and p40, both of which are required for the secretion of the active form of IL-12, p70. Interleukin-12 acts on dendritic cells (DC), leading to increased maturation and antigen presentation, which can allow for the initiation of a T cell response to tumor specific antigens. It also drives the secretion of IL-12 by DCs, creating a positive feedback mechanism to amplify the response. Once a response is initiated, IL-12 plays a fundamental role in directing the immune system towards a Th1 cytokine profile, inducing CD4⁺ T cells to secrete interferon-gamma (IFN-γ) and leading to a CD8⁺ cytotoxic T cell response.⁴ However, IL-12 is also a strong pro-inflammatory cytokine that leads to the secretion of other cytokines including tumor necrosis factor-alpha (TNF-α) which, combined with IFN-γ, is a prerequisite for the development of CD4⁺ cytotoxic T lymphocytes (CTL).⁵ Furthermore, IL-12 can promote the activation of innate immune cells such as macrophages and eosinophils through its induction of IFN-γ and other cytokines. This activation then leads to IL-12 secretion by these cells and further amplification of both the innate and acquired responses.⁴ However, high levels of IL-12, and consequently IFN-γ, have also been associated with induction of antagonistic molecules such as IL-10 and the depletion of signalling molecules downstream of IL-12, such as STAT4.⁶⁻⁸

Innovative gene therapy strategies may accelerate the development of prophylactic immunotherapy against cancer.

### SUMMARY

The application provides compositions for delivering tumor associated antigens and immune modulatory molecules to result in a therapeutic effect. In one embodiment, the composition comprises a stably integrated delivery vector, a tumor associated antigen cassette and a lysosomal targeting cassette, which is operably linked to the tumor associated antigen cassette.

In one aspect of the application, the delivery vector comprises a retroviral vector, optionally a lentiviral vector. In one embodiment, the lentiviral vector comprises one or more of a: 5′-Long terminal repeat (LTR), HIV signal sequence, HIV Psi signal 5′-splice site (SD), delta-GAG element, Rev Responsive Element (RRE), 3′-splice site (SA), Elongation factor (EF) 1-alpha promoter and 3′-Self inactivating LTR (SIN-LTR). In another embodiment, the lentiviral vector comprises a central polypurine tract and/or a woodchuck hepatitis virus post-transcriptional regulatory element; optionally wherein the cPPT comprises SEQ ID NO:2 and/or the WPRE comprises SEQ ID NO:3; or, optionally wherein the cPPT comprises at least 70% sequence identity to SEQ ID NO:2 and/or a the WPRE comprises at least 70% sequence identity to SEQ ID NO:3. In yet another embodiment, the lentiviral vector comprises the nucleotides corresponding to a pHR′ vector backbone. In yet a further embodiment, the delivery vector is a clinical grade vector.

In another aspect of the application, the tumor associated antigen cassette comprises all or part of a carcinoembryonic antigen polynucleotide. In one embodiment, the carcinoembryonic antigen polynucleotide comprises at least 70% sequence identity to a sequence selected from the group comprising CEA sequences in the application or incorporated by reference herein.

In another aspect of the application, the tumor associated antigen cassette comprises all or part of a HER-2/neu polynucleotide. In one embodiment, the carcinoembryonic antigen polynucleotide comprises at least 70% sequence identity to a HER-2/neu polynucleotide.

In another aspect of the application the lysosomal targeting cassette comprises a LAMP1 lysosomal targeting polynucleotide. In one embodiment the LAMP1 lysosomal targeting polynucleotide is selected from the group consisting of SEQ ID NO:1 or a polynucleotide having at least 70% sequence identity to SEQ ID NO:1 which maintains lysosomal targeting activity.

In aspect of the application, the composition of the disclosure comprising an activator polynucleotide encoding a polypeptide that converts a prodrug to a drug, optionally a modified tmpk polynucleotide and/or a tmpk polynucleotide with at least 80% sequence identity to a modified tmpk polynucleotide described herein.

In another aspect of the application, a composition of the disclosure comprises a detection cassette. In a further aspect, the detection cassette is selected from the group consisting of CD19, truncated CD19, CD20, human CD24, murine HSA, human CD25 (huCD25), a truncated form of low affinity nerve growth factor receptor (LNGFR), truncated CD34 or erythropoietin receptor (EpoR) polynucleotides and/or a polynucleotide comprising at least 70% sequence identity to a CD19, truncated CD19, CD20, human CD24, murine HSA, CD25, a truncated form of low affinity nerve growth factor receptor (LNGFR), truncated CD34 or erythropoietin receptor (EpoR)polynucleotide.

In another aspect of the application, the composition of the disclosure comprises an immune modulatory cassette. In another embodiment, the immune modulatory cassette comprises a polynucleotide selected from the group comprising IL-12 p35, IL-12 p40, IL-12 fusion, IL-15, RANKL, CD40L, IFNg and TNFα polynucleotides and combinations thereof. In a further embodiment, the immune modulatory cassette encodes a protein that modulates dendritic cells or a protein that modulates T cells, optionally CD4+ T cells. In yet a further embodiment, the immune modulatory cassette comprises IL-12 fusion polynucleotide, optionally a mammalian IL-12 polynucleotide. Optionally, the IL-12 polynucleotide comprises at least 70% sequence identity to a sequence described herein.

In another aspect of the application, the composition of the disclosure is a pharmaceutical composition and further comprises a pharmaceutically acceptable carrier.

The application also provides a vector construct to be used in accordance with the disclosure. In one embodiment, the vector construct comprises a stably integrating delivery vector, a tumor associated antigen cassette, and a lysomal targeting cassette, wherein the lysosomal targeting cassette is operatively linked to the tumor associated antigen cassette.

In another aspect of the application, an isolated virus, optionally a lentivirus, comprising the vector construct or the composition of the disclosure is provided,

The application also relates to an isolated cell transduced with the composition of the disclosure, the vector construct of the disclosure, or the virus of the disclosure. The cell is optionally an antigen presenting cell, optionally a stem cell, immune cell, hematopoietic cell, dendritic cell, or an immature dendritic cell and/or a population of cells comprising the isolated cell.

Another aspect of the application relates to methods for delivering tumor associated antigens and immune modulatory molecules to result in a therapeutic effect. In one embodiment, the method relates to expressing a tumor associated antigen in a mammalian cell comprising contacting the mammalian cell with a composition of the disclosure, a vector construct of the disclosure, or a virus of the disclosure. In one embodiment, the transduced cell is growth arrested or irradiated prior to administering to the subject. In another embodiment, the virus or cells are introduced by intravenous injection, IP injection, subcutaneously or intradermally.

Another aspect of the application relates to a method of expressing a tumor associated antigen and an immune modulatory protein in a mammalian cell comprising contacting the mammalian cell with a composition of the disclosure, a vector construct of the disclosure, or a virus of the disclosure.

Another aspect of the application relates to a method of expressing a tumor associated antigen, an immune modulatory polynucleotide, an activator polynucleotide and a targeting polynucleotide in a mammalian cell comprising contacting the mammalian cell with a composition of the disclosure a vector construct of the disclosure or a virus of the disclosure.

In an aspect of the application, the method includes expression in a mammalian cell selected from the group consisting of a stem cell, an immune cell, a hematopoietic cell, an antigen presenting cell, a cancer cell and a dendritic cell. In one embodiment, the transduced cell is a dendritic cell, optionally, an immature dendritic cell. In another embodiment, the cell transduced is a subject autologous dendritic cell. In another embodiment, the method of expressing a cell further comprises a step of detecting expression of the tumor associated antigen in the transduced cell. In yet another embodiment, the tumor associated antigen expression is detected in a lysosomal fraction of the transduced cell. In a further embodiment, the method further comprises a step of treating the transduced cell with a cell maturing agent. Optionally, the cell maturing agent is TNFα. In yet a further embodiment, the method further comprises a step of isolating the transduced cells or a step wherein the isolated mammalian cell is transplanted in a mammal.

Another aspect of the application relates to a method of treating a subject in need thereof, optionally a subject with cancer or an increased risk of developing cancer, comprising administering to the subject in need thereof a composition the disclosure, a vector of the disclosure, a virus of the disclosure, or the traduced cell or population cells of the disclosure. Optionally, the transduced cell is an antigen presenting cell and the cancer is colon cancer, rectal cancer, stomach cancer, pancreatic cancer, non-small cell lung cancer, metastatic pancreatic cancer, ovarian cancer or breast cancer. In one embodiment, the transduced cell is a dendritic cell, optionally, an immature dendritic cell. In another embodiment, the cell transduced is a subject autologous dendritic cell.

Another aspect of the application is a method of reducing cancer burden in a subject having a CEA or HER-2/neu positive cancer comprising vaccinating the subject with a composition of the disclosure, a vector construct of the disclosure, a virus of the disclosure, or a transuded cell or population of cells of the disclosure. In one embodiment, the transduced cell is a dendritic cell, optionally, an immature dendritic cell. In another embodiment, the cell transduced is a subject autologous dendritic cell. In one embodiment, the method further comprises a step of monitoring cancer progression. Optionally, the cancer is colon cancer, rectal cancer, stomach cancer, pancreatic cancer, non-small cell lung cancer, metastatic pancreatic cancer, ovarian cancer or breast cancer.

Another aspect of the application relates to a method of inducing or enhancing an immune response in a subject in need thereof comprising administering to the subject a composition of the disclosure, a vector construct of the disclosure, an isolated virus of the disclosure, or a transduced cell or population of cells of the disclosure. In one embodiment, the immune response comprises a CD4+ mediated immune response.

Another aspect of the application relates to a method of inducing or enhancing a memory immune response in a subject in need thereof comprising administering to the subject in need thereof a composition of the disclosure, a vector construct of the disclosure, an isolated virus of the disclosure, or a transduced cell or population of the disclosure. In one embodiment, the immune response comprises a CD4+ mediated immune response.

Another aspect of the application relates to uses of a composition of the disclosure, a vector construct of the disclosure, or a transduced cell or a population of cells of the disclosure. In one embodiment, the use is treating a subject in need thereof, optionally a subject with cancer or an increased risk of developing cancer. In another embodiment, the use is reducing cancer burden in a subject having a CEA or HER-2/neu positive cancer. In another embodiment, the use for reducing cancer burden, further comprising a step of monitoring cancer progression. In yet another embodiment, the cancer is colon cancer, rectal cancer, stomach cancer, pancreatic cancer, non-small cell lung cancer, metastatic pancreatic cancer, ovarian cancer or breast cancer. In yet a further embodiment, the transduced cell is a dendritic cell. Optionally, the dendritic cell is an immature dendridic cell or a subject autologous dendritic cell. In yet another embodiment, the transduced cell is growth arrested or irradiated. In yet a further embodiment, the cells are suitable for intravenous injection, IP injection, subcutaneous administration or intradermal administration.

Another aspect of the application relates to the use of a composition, vector construct, virus, transduced cell or population of cells of the disclosure for inducing or enhancing an immune response in a subject in need. Another aspect relates to the use of a composition, vector construct, transduced cell or population of cells of the disclosure for inducing or enhancing a memory immune response in a subject in need thereof. In one embodiment, the immune response comprises a CD4+ mediated immune response. In yet another embodiments, the transduced cell is growth arrested or irradiated. In a further embodiment, the cells are suitable for intravenous injection, IP injection, subcutaneous administration or intradermal administration.

Another aspect of the application relates to the use of a composition, a vector construct, a virus, a transduced cell or population of cells of the disclosure for manufacture of a medicament for treating a subject in need thereof, optionally a subject with cancer or an increased risk of developing cancer. In another aspect of the application a composition, vector construct, virus, transduced cell or population of transduced cells of the disclosure are used for manufacture of a medicament for reducing cancer burden in a subject having a CEA or HER-2/neu positive cancer.

Another aspect of the application relates to the use of a composition, vector construct, virus, transduced cell or population of cells of the disclosure for manufacture of a medicament for enhancing an immune response in a subject in need thereof.

A further aspect of the application provides the use of a composition, vector construct, virus, transduced cell or population of cells of the disclosure for manufacture of a medicament for inducing or enhancing a memory immune response in a subject in need thereof.

In further embodiments of the application, the methods and uses of the application involve administering cells of the disclosure, wherein the number of cells administered ranges from 10⁵ cells to 10⁹ cells, optionally about 10⁵ cells, about 10⁶ cells, about 10⁷, cells, about 10⁸ cells, or about 10⁹ cells.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE FIGURES

The following non-limiting examples are illustrative of the present invention:

FIG. 1. A) Flow cytometry and B) Western blot confirmation of antibody specificity for CEA (180 kDa). The positive control LoVo is a human colon cancer cell line overexpressing CEA. 293T cells do not express CEA.

FIG. 2. A shows the induction of CEA expression by the transduction (right) compared to the basal level of CEA on NT cells (left). B shows that DCs marker expression is not affected. C shows a higher CD86 expression in transduced cells.

FIG. 3. Schematic diagram of lentiviral vector (LV) constructs used in these studies. LTR, long-terminal repeat; SD, splice donor site; SA, splice acceptor sites; ψ, RNA packaging signal; EF1α, elongation factor 1 α promoter; SIN LTR, Self-inactivating LTR; CMV, cytomegalovirus promoter; Gag, viral structural proteins; RRE, Rev response element.

FIG. 4. Transduction efficiency of LV/erb and LV/enGFP on DCs. BM cells were cultured in the presence of GM-CSF and IL-4. DC maturation was induced with TNF-α on culture day 8. Transductions were performed on culture day 3 using either erbB2tr- or enGFP-encoding lentiviruses. Transgene expression on DCs used for the first (A) and the second (B) scheduled immunizations was monitored by flow cytometry. Numbers indicate the percentage of transgene-positive cells for each histogram.

FIG. 5. Transducing DCs with LV/erb and LV/enGFP does not affect DC phenotype or allostimulatory capacity. BM-derived DCs were transduced on culture day 3 and matured on culture day 8. Transduced and non-transduced (NT) immature (day 5 and/or 7) and mature (day 9) DCs prepared for the first (A) and the second (B) immunizations were stained with antibodies recognizing CD11c, I-A^{b}, CD80, and CD86 and analyzed by flow cytometry. Numbers indicate the percentage of positive cells in the indicated density plot quadrants. Mature day 9 DCs from C57BL/6 mice were also co-cultured with [³H]thymidine-pulsed syngeneic (C57BL/6) or allogeneic (BALB/c) splenocytes to compare the allostimulatory capacity of non-transduced DCs to either (C) erbB2tr- or (D) enGFP-transduced DCs. Co-cultures were plated in triplicate and the mean±SD values are shown.

FIG. 6. Immunization with low doses of DC-erbB2tr generates potent anti-tumor responses. Cell lines RM1-erbB2tr and non-transduced RM1 (RM1-NT) were stained with anti-erbB2 antibody and analyzed by flow cytometry (A). Mice were immunized twice with either 2×10⁵ (B) or 2×10³ (C) DCs and then inoculated with RM1-erbB2tr and RM1-NT cells on opposite flanks. Plotted values are the mean±SEM of 5 or 6 mice per group, except for the positive control group (n=3). *P<0.05, vs DC-erbB2tr vaccination; **P<0.005, vs DC-erbB2 vaccination.

FIG. 7. DC-erbB2tr immunization dose of 2×10⁵ cells causes a sustained erbB2-specific humoral response. Plasma samples from naïve and immunized mice were pooled according to cohort and quantitatively tested for the presence of anti-erbB2 antibodies by a flow cytometry-based ELISA. Graphs show a comparison between anti-erbB2tr levels from control groups and either (A) 2×10⁵ or (B) 2×10³ DC dose groups. Plotted values are the mean±SEM of three independent assays. *P<0.01, vs vaccination with DC-NT, DC-enGFP, or no vaccination.

FIG. 8. Immunization using DCs transduced with LV-erb offers antigen-specific Th1 immunity. Splenocytes from naïve and immunized mice were co-cultured in triplicate with LV-transduced or non-transduced DCs for 24 hours and supernatants were analyzed for IL-2, IFN-γ, and TNF-α by Bio-Plex multiplex sandwich immunoassays. Specificity index values were calculated by normalizing cytokine concentration values from each 24-hr re-stimulation condition to the values obtained from re-stimula

FIG. 9. Representative tumors growth curves after subcutaneous injection of 0.6 million cells to huCEA transgenic mice (A) mGC8 cells (dark curves) or mGC4CEA cells (light hatched curves) (B) Representative tumor size after injection of cells in huCEA transgenic mice.

FIG. 10. PB-derived human DCs were stained with specific markers of these cells (A) and infected with the LV/huCEA with an MOI of 8.8 (B, right) or not (B, left) and stained with an antibody against CEA conjugated to PE and an antibody against HLA-DR FITC-conjugated (FL-1).

FIG. 11. Immunization with LV-huCEA induces CEA-expressing tumor regression. In this representative experiment, CEA transgenic mice were subcutaneously injected with mGC4CEA cells in the flank. Mice were then vaccinated in the footpad with either PBS (5 mice, empty squares) or LV-huCEA (6 mice, filled circles) or LV-enGFP (5 mice, empty diamonds). Tumors were measured over time by using a caliper.

* P<0.005 compared to PBS and GFP controls

FIG. 12. Immunization with LV-huCEA induces anti-huCEA antibody secretion in huCEA transgenic mice sera. In these experiments, huCEA transgenic mice bearing mGC4CEA tumors were vaccinated with either PBS (5 and 4 mice in A and B respectively, empty squares) or LV-huCEA (6 mice in A and 5+4 mice in B, filled circles and triangles) or LV-enGFP (5 mice in both A and B experiments, empty diamonds). Anti-huCEA antibodies were titered by ELISA. Fold induction were calculated relative to the value of the pre-immune serum.

FIG. 13. Immunization with LV-huCEA induces a general immune response. In these tumor rejection experiments, splenocytes from huCEA transgenic mice sacrificed at day 28 were cultured and secreted cytokines were detected by luminex at 24 (open bars) or 48 h (shaded bars). Part A, B, C, and D represent the fluorescence intensity (FI) measured for IFN-γ IL-4, IL-2, and IL-10 detection respectively. Left histograms show data from the first experiment. Right histograms show data from the second experiment.

### * P<0.05; ** P<0.005; *** P<0.0005 (for CEA vs GFP)

FIG. 14. Quantification of specific IFN-γ secretion in the presence of huCEA-expressing target cells. At the end of the second tumor rejection experiment, pools of splenocytes from CEA transgenic mice were cultured alone (open bars) or with mGC4CEA cells (shaded bars). For the co-cultures, cells were cultured with an effector:target ratio of 20:1 and IFN-γ secretion was measured by ELISA.

** P<0.005 compared to PBS and GFP controls; * P<0.05 for CEA with mGC4CEA compared to without mGC4CEA.

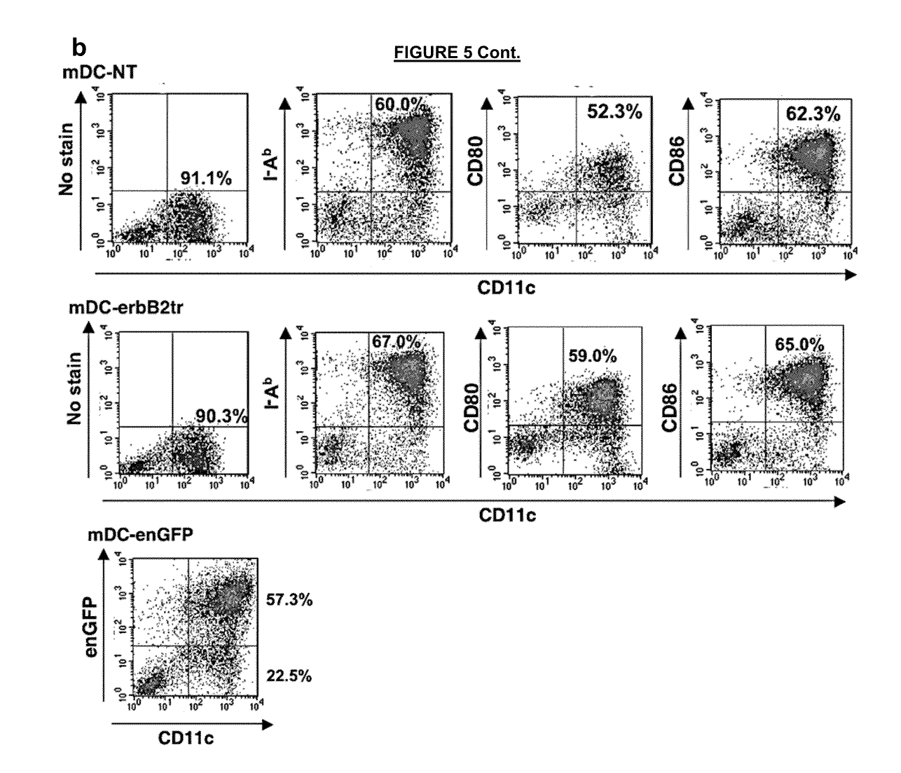

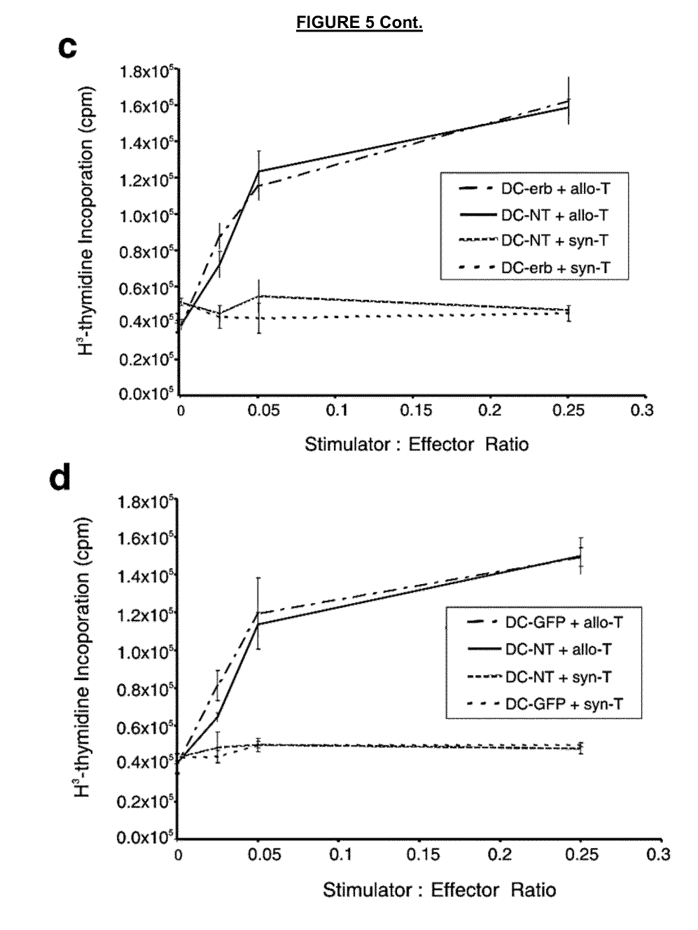

FIG. 15. Immunization with LV-huCEA induces the production of a CD8⁺ population specific for a CEA peptide. After the second tumor rejection experiment, pooled splenocytes from huCEA transgenic mice were stained either with a APC-conjugated anti-CD8 antibody and a PE-conjugated CEA/H-2Db-tetramer (shown here) or with the matched isotype controls (flat curves, not shown). FIG. 5 represents the number of cells relative to the FL-2 fluorescence, gated on CD8⁺ cells. The continuous thick line is the curve representative for splenocytes from LV-huCEA-vaccinated mice, whereas thin dotted lines correspond to the staining of the control groups splenocytes.

FIG. 16. Immunization with LV-huCEA induces CD8⁺ and CD4⁺ cell infiltrates in tumors. After the second tumor rejection experiment, huCEA transgenic mice tumors were harvested. Sections were made and stained with DAPI as well as either an anti-CD4 antibody or an anti-CD8 antibody. For each area, the ratios between the positive area fractions obtained with Alexafluor488 and DAPI was evaluated using imagej software and designated “AD ratios”. Average was calculated per mouse and then the background was deduced by substracting the ratio obtained for the negative control. The result was designated as “absolute AD ratios”. FIG. 5A represents the “absolute” AD ratios obtained in the different groups. FIG. 5B shows pictures of different patterns of DAPI and Alexafluor488 overlays obtained in one PBS-vaccinated mouse and one LV-huCEA vaccinated mouse after no primary, anti-CD4 or anti-CD8 stainings.

FIG. 17. The anti-tumor immune response induced by vaccination with LV-huCEA does not persist long-term. In the second tumor rejection experiment, one group of LV-huCEA-vaccinated mice received a third vaccination at day 28 with a lower dose (0.1×10⁶ TU) and was followed for 51 days. Similar immuno-analyses were performed on two mice sacrificed at day 57. Naïve mice were used as controls. Part A shows the tumor sizes data, part B represents the antibody response, and part C shows the specific IFN-γ secretion. *** P<0.005 for CEA compared to naïve mice.

### DETAILED DESCRIPTION

The application describes novel lentivrial constructs comprising a tumor associated antigen (TAA) cassette for expressing a tumor associated antigen or a fragment or variant thereof and methods for therapeutically and prophylacticly treating a subject with a tumor expressing the tumor associated antigen encoded by the tumor associated antigen cassette.

The inventors have utilized a recombinant lentiviral vector construct that engineers expression of tumor associated antigens such as human carcinoembryonic antigen (CEA) to transduce dendritic cells (DCs) for use as novel cancer prophylactic and therapeutic vaccines.

The inventors have also synthesized a novel lentiviral vector construct encoding a kinase-deficient form of erbB2 (erbB2tr) to efficiently transduce DCs. Murine erbB2 models a clinically relevant tumor-associated self-antigen; its human homolog (HER-2/neu) is overexpressed in breast cancer and 80% of metastatic prostate cancers.

The inventors disclose vector constructs capable of targeting TAA to the lysosomes of transduced cells. Such vector constructs are useful for transducing antigen presenting cells such as dendritic cells. Optimizing organelle specific antigen localization can improve loading and presentation of TAA peptides which can affect MHC Class I versus Class II stimulation. Lysosomal targeting of TAA in TAA transduced antigen presenting cells such as dendritic cells is useful for augmenting the CD4+ arm of the immune response and increasing the anti-tumor response against tumors expressing the TAA. The inventors also disclose lentiviral constructs comprising an IL-12 cassette in combination with a TAA cassette and optionally a lysosome targeting sequence cassette and transduction of APC with such vector constructs to produce APCs expressing IL-12 and TAA which are useful for tumor immunotherapy. The combined expression is optionally obtained using multicistronic lentiviral constructs expressing a TAA and IL-12 or separate constructs which are simultaneously introduced into the host cell.

### I. DEFINITIONS

The term “a cell” as used herein includes a plurality of cells.

The term “allogenic” also referred to as “allogeneic” as used herein means cells, tissue, DNA, or factors taken or derived from a different subject of the same species. For example in the context where allogenic transduced cancer cells are administered to a subject in need thereof, cancer cells removed from a subject that is not the subject, are transduced or transfected with a vector that directs the expression of IL-12 and the transduced cells are administered to the subject. The phrase “directs expression” refers to the polynucleotide comprising a sequence that encodes the molecule to be expressed. The polynucleotide may comprise additional sequence that enhances expression of the molecule in question.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include without limitations Fab, Fab′, F(ab′)2, scFv, dsFv, ds-scFv, dimers, minibodies, diabodies, and multimers thereof, multispecific antibody fragments and Domain Antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques. The term also includes antibodies or antibody fragments that bind to the detecting cassette polypeptides disclosed herein.

The term “autologous” as used herein refers to cells, tissue, DNA or factors taken or derived from an individual's own tissues, cells or DNA. For example in the context where autologous transduced cancer cells are administered to a subject in need thereof, cancer cells removed from the subject are transduced or transfected with a vector construct and the transduced cells are administered to the subject.

The term “cancer” as used herein means any proliferative disorder wherein the cells abnormally divide and optionally invade or spread to nearby and/or distant tissues. Cancers of virtually every tissue are known. The phrase “cancer burden” refers to the quantum of cancer cells or cancer volume in a subject. Reducing cancer burden accordingly refers to reducing the number of cancer cells or the cancer volume in a subject.

The phrase “cancer that is characterized by periods of remission” refer to cancers that may respond to a treatment but wherein the cancer recurs at some later time suggesting that not all cancer cells were eradicated by the treatment.

The term “cancer cell” as used herein refers to any cell that is a cancer cell or is derived from a cancer cell e.g. clone of a cancer cell.

The term “cassette” as used herein refers to a polynucleotide sequence that is to be expressed. The cassette can be inserted into a vector. The cassette optionally includes regulatory sequence to direct or modify its expression.

The phrase “cell surface protein” or “cell surface polypeptide” as used herein refers to a polypeptide that is expressed, in whole or in part on the surface of a cell. This optionally includes polypeptide fragments that are presented on cells as well as polypeptides or fragments thereof that are naturally found on the surface of a cell. In the context of a cell modified to express a vector construct comprising a detection cassette polypeptide, wherein the detection cassette polypeptide is a cell surface polypeptide, the cell surface marker need not be native to the cell it is being expressed on.

The term “clinical grade vector” as used herein refers to a method of preparing a virus or vector such that it is prepared under near-GMP and GMP (good manufacturing practice) conditions and is quality assurance certified free of any contamination (including bacteria, other viruses,) and produced at a defined level of purity, titre, safe for injection into humans.

A “conservative amino acid substitution” as used herein, is one in which one amino acid residue is replaced with another amino acid residue without abolishing the protein's desired properties. Conservative amino acid substitutions are known in the art. For example, conservative substitutions include substituting an amino acid in one of the following groups for another amino acid in the same group: alanine (A), serine (S), and threonine (T); aspartic acid (D) and glutamic acid (E); asparagine (N) and glutamine (Q); arginine (R) and lysine (L); isoleucine (I), leucine (L), methionine (M), valine (V); and phenylalanine (F), tyrosine (Y), and tryptophan (W).

The term “detection cassette” as used herein refers to a polynucleotide that directs expression of a molecule that is useful for enriching, sorting, tracking and/or killing cells in which it is expressed. The detection cassette encodes a polypeptide that is expressed in the transduced or transfected cell and can as a result be used to detect and/or isolate transduced or transfected cells. The detection cassette is optionally used to determine the efficiency of cell transduction or transfection.

As used herein, the phrase “effective amount” or “therapeutically effective amount” or a “sufficient amount” of a compound or composition of the present application is a quantity sufficient to, when administered to the subject, including a mammal, for example a human, effect beneficial or desired results, to prevent or treat a disorder, such as cancer. Also, as used herein, a “therapeutically effective amount” of a compound of the present disclosure is an amount to prevent or treat a disorder in a subject, such as cancer, as compared to a control. As defined herein, a therapeutically effective amount of a compound of the present disclosure may be readily determined by one of ordinary skill by routine methods known in the art. Dosage regime may be adjusted to provide the optimum therapeutic response.

An “immune modulatory cassette” as used herein, means a polynucleotide that directs expression of a molecule or polypeptide that enhances or augments the anti-tumor immune response.

The term “immune response” as used herein refers to activation of the immune system (either cellular or humoral) directed against one or more specific antigens. The phrase “inducing an immune response” as used herein refers to a method whereby an immune response is activated. The phrase “enhancing an immune response” refers to augmenting an existing but immune response.

The term “immunotoxin” as used herein means an antibody or fragment thereof that is cytotoxic and/or an antibody or fragment thereof that is fused to a toxic agent.

The term “increased risk of cancer” as used herein means a subject that has a higher risk of developing a particular cancer than the average risk of the population. A subject may have a higher risk due to previously having had said particular cancer and/or having a genetic risk factor for said particular cancer and/or other risk factors.

The term “Lysomal targeting sequence” as used herein means any nucleotide or protein sequence, naturally occurring or non-naturally occurring, that, when linked to a molecule that is not normally targeted to the lysosome, directs that molecule to the lysosome.

The term “polynucleotide” and/or “nucleic acid sequence” as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine.

The term “polypeptide” as used herein refers to a sequence of amino acids consisting of naturally occurring residues, and/or non-naturally occurring residues. The term “polypeptide” and “peptide” are used interchangeably herein.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). The percent identity between two sequences can be determined using techniques similar to those described above, with or without allowing gaps. In calculating percent identity, typically only exact matches are counted.

The term “subject” as used herein includes all members of the animal kingdom including mammals, suitably humans. The term subject also refers to patients.

The term “subject in need thereof” refers to a subject that could benefit from the method, and optionally refers to a subject in need thereof, such as a subject with cancer, or optionally a subject with increased risk of cancer, such as a subject previously having cancer, a subject with a precancerous syndrome or a subject with a strong genetic disposition.

The term “transduction” as used herein refers to a method of introducing a vector construct or a part thereof into a cell. Wherein the vector construct is comprised in a virus such as for example a lentivirus, transduction refers to viral infection of the cell and subsequent transfer and integration of the vector construct or part thereof into the cell genome.

The term “treating” or “treatment” as used herein means administering to a subject a therapeutically effective amount of the compositions, cells or vector constructs of the present application and may consist of a single administration, or alternatively comprise a series of applications.

As used herein, and as well understood in the art, “treatment” or “treating” is also an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment. Further any of the treatment methods or uses described herein can be formulated alone or for contemporaneous administration with other agents or therapies.

“Tumor associated antigen” as used herein means an antigen that is expressed by tumors in greater amounts than normal cells. Examples of tumor associated antigens include carcinoembryonic antigen (CEA), prostate-specific antigen (PSA), prostate-specific membrane antigen (PSMA), melanoma associated MART-1 and HER-2/neu.

The term “Tumor associated antigen cassette” or “TAA cassette” as used herein means a polynucleotide that encodes a tumor associated antigen, a fragment or variant thereof. A fragment can be any length of a polynucleotide that encodes an antigenic peptide.

The term “vector construct” alternatively referred to as “construct” as used herein refers to a recombinant polynucleotide comprising a vector, alternatively referred to as a vector backbone, and at least one coding cassette. A vector construct is optionally comprised in a virus, such as a lentivirus. The term “vector” has used herein refers to a means by which polynucleotides can be introduced into a cell or host.

### II. VECTOR CONSTRUCTS AND VIRUS

The application provides in one aspect a vector construct or virus comprising the vector construct, the vector construct comprising a delivery vector and a tumor associated antigen (TAA) cassette.

### a) Tumor Associated Antigens (TAA)

Tumor cells often express antigens that highly correlate with the tumor cell and which are not normally associated with normal cells or are expressed at much lower levels in normal cells. Examples of tumor associated antigens include carcinoembryonic antigen (CEA), prostate-specific antigen (PSA), prostate-specific membrane antigen (PSMA), melanoma associated MART-1 and HER-2/neu.

In certain embodiments, the TAA cassette encodes a fragment of a TAA that is antigenic. A fragment can be any length of a polynucleotide that encodes an antigenic peptide. Dendritic cells present peptides that are typically 8-12 amino acids. In one embodiment the TAA cassette comprises a polyncucleotide that encodes a peptide of at least 8 amino acids that is antigenic. Preferably multiple peptides are presented. In a preferred embodiment the tumor associated antigen cassette comprises a polynucleotide that encodes a substantial portion (e.g. more than 20%, more than 30% more than 40%, more than 50%, more than 60%, more than 70%, more than 80%, more than 90%) or the full length protein. In another embodiment, the TAA cassette encodes a TAA variant. A variant of a tumor associated antigen comprises any modified TAA that is capable of eliciting an immune response to the unmodified TAA. In one embodiment, the variant comprises one or more conservative amino acid substitutions.

i. Carcinoembryonic Antigen

Carcinoembryonic Antigen (CEA) is an oncofetal protein expressed normally during fetal development. Human CEA belongs to a family of 29 genes of which 18 are expressed. The family consists of three subgroups: the CEA subgroup and the pregnancy specific glycoprotein subgroup. Of the 18 expressed genes, 7 belong to the first and 11 belong to the latter subgroup. A third subgroup comprises 6 pseudogenes. (Hammarstrom S. Seminars in Cancer Biology 9:67-81 1999). CEA is expressed at low levels in normal gut epithelium but may be upregulated in colon cancer, non-small cell lung cancers, breast cancers, gastric cancers, prostate cancer, pancreatic cancers and cancers of the small intestine. CEA is expressed on the majority of colon, rectal, stomach and pancreatic tumors (Muraro et al., Cancer Res., 45:5769, 1985) as well as in breast (Steward et al., Cancer, 33:1246, 1974); and lung (Vincent and Chu, J. Thor. Cardiovasc. Surg., 66:320, 1978) tumors.

Clinically, it is used as a screening tool for the detection and treatment of early recurrent or metastatic disease. CEA is a favourable tumor-associated antigen (TAA) for use in immunotherapeutic strategies because: 1) it is over-expressed in colon cancer and other cancers but minimally in normal tissues; 2) it is not lost as tumours progress, in fact it is often further up-regulated in metastatic disease; 3) it is expressed both on the tumour cell surface and as a soluble protein; 4) the ability to overcome self tolerance to CEA; 5) commercially available kits (ELISA, antibodies) are available for its detection.

Accordingly, in one embodiment the TAA cassette encodes a CEA. Human CEA is in one embodiment encoded by the cDNA disclosed in Genbank accession number: M17303, E01630 or M29540 (18022). In this sequence, the ATG start codon is at position 115 and the stop codon TAG is at 2223.

The complete cDNA sequence encoding the human CEA protein codes for a polypeptide of 702 amino acids (Beauchemin N., Benchimol S., Cournoyer D., Fuks A. and Stunners C. P. “Isolation and characterization of full-length functional cDNA clones for human carcinoembryonic antigen”, Mol. Cell. Biol., 7, 3221-3230, 1987). In one embodiment the TAA cassette comprises a polynucleotide encoding the full-length protein of a CEA. CEA molecules have multiple antigenic epitopes that are known in the art. Minimally a single antigenic fragment or epitope of CEA is useful. In one embodiment the TAA cassette comprises a polynucleotide encoding a fragment of a CEA. The polynucleotide encoding the fragment is minimally 24 nucleotides of the coding sequence of a CEA. In one embodiment the TAA cassette comprises a polyncucleotide that encodes a peptide of at least 8 amino acids that is antigenic. The polynucleotide is optionally, 24-50, 50-100, 100-200 amino acids. Suitable epitopes and peptides are disclosed in EP1561817, U.S. Pat. No. 6,602,510, WO0142270, and WO2004055183 which are incorporated herein by reference. In another embodiment, the TAA cassette encodes a CEA variant. In one embodiment the CEA variant comprises one or more conservative amino acid substitutions. Sequences having at least: 70%, 80%, 90%, 95%, 98% or 99% identity to the CEA human cDNA or polypeptide are also useful. Sequence identity is optionally measured using BLAST (default parameters).

ii. HER-2/neu

HER-2/neu is the human homolog of murine erbB2 antigen. HER-2/neu is overexpressed in 20% of primary prostate tumors and 80% of patients with metastatic prostate cancer, making this TAA a clinical target for immunotherapy [17]. HER-2/neu is also overexpressed in other malignancies including breast, ovarian, and lung tumors [18-20]. A naturally occurring kinase-truncated variant of HER-2/neu has also been described in human tumor cells [21]. Accordingly in one embodiment, the TAA cassette encodes a HER-2/neu. In another embodiment, the TAA cassette encodes a fragment thereof. In a further embodiment, the TAA cassette encodes a variant of HER-2/neu. In certain embodiments, the variant comprises one or more conservative amino acid substitutions.

### b) Lysosomal Targeting of Tumor Associated Antigens.

Augmenting the CD4⁺ T cell response enhances the antitumor effect against TAA-expressing tumor cells such as CEA-expressing tumor cells or HER-2/neu expressing tumor cells. Enhanced CD4+ T cell responses are augmented by targeting a TAA expressed in an APC to APC lysosomes. Lysosome targeting is accomplished using a lysosomal targeting sequence to redirect the translated TAAs such as a CEA or HER-2/neu product into the Class II presentation pathway.

Accordingly, in certain embodiments, the composition or vector construct comprises a lysosomal targeting sequence cassette. Lysosome targeting sequences are found in proteins that are directed to lysosomes and include proteins alpha-galactosidase A, beta-glucoronidase, glucocerebrosidase and acid ceramidase and other lysomla hydrolases. In a preferred embodiment the lysosome targeting sequence cassette comprises the lysosome targeting sequence of alpha-galactosidase A. The amino acid sequence of the alpha-galactisidase A lysosomal targeting sequence is reported in Bishop et al. 1987 (SEQ ID NO: 1). The lysosome targeting sequence cassette is operatively linked to the TAA cassette such that an in frame fusion is produced is produced.

Accordingly in one embodiment, the composition or vector construct comprises a lysosmal targeting cassette. In another embodiment, the TAA cassette is fused or operatively linked to a lysomal targeting cassette. In certain embodiments, the lysosomal targeting cassette comprises a variant of a lysosomal targeting sequence. In certain embodiments, the variant comprises one or more conservative amino acid substitutions. In a further embodiment, the lysosomal targeting cassette comprises SEQ ID NO:1 or a variant polynucleotide with at least 70%, 70-80%, 80-90%, 90-95%, 95-99%, or 99-99.9% sequence identity to SEQ ID NO:1.

### c) Immune Modulatory Cassette

Enhanced anti-tumor effect is obtainable with the use of specific immune modulatory molecules. One class of immune regulatory molecules is cytokines. Cytokines are integral to both the innate and acquired immune systems. They can alter the balance of cellular and humoral responses, alter class switching of B lymphocytes and modify innate responses. These traits have made a number of cytokines interesting candidates for cancer innnnunotherapies.¹⁻³ Among these, IL-12 has been tested for its ability to promote immune recognition and response against tumors.

i. Interleukin-12 (IL-12)

Interleukin-12 is a heterodimeric cytokine with multiple biological effects on the immune system. It is composed of two subunits, p35 and p40, both of which are required for the secretion of the active form of IL-12, p70. Interleukin-12 acts on dendritic cells (DC), leading to increased maturation and antigen presentation, which can allow for the initiation of a T cell response to tumor specific antigens.

In one embodiment the immune modularity cassette comprises a polynucleotide that expresses IL-12. In one embodiment the polynucleotide comprises the sequence of both IL-12 subunits separated by an IRES sequence which permits expression of multiple transgenes from a single transcript. In other embodiments, the IL-12 is a fusion molecule that retains IL-12 activity.

ii. Other Cytokines

A second cytokine that is useful for promoting anti-tumor effect is RANKL. RANKL is a molecule that extends the lifespan of DCs in an autocrine fashion.

CD40L which enhances the stimulatory capacity of DCs, is also useful for promoting the anti-tumor effect of DC and tumor cell vaccines. TNFα is also useful as it promotes DC maturation. Further IFN-γ and IL-7 are also useful. Interleukin 15 (IL-15) is a cytokine with strong anti-tumor properties and has potential use in tumor immunotherapy. IL-15 exerts its effect on innate and acquired immunity with the most prominent action in NK cells and CD8+ memory T cells. Other useful immunomodulatory cytokines include TNF alpha, type I and II IFNs, IL 2, IL12, IL 15 and IL 18. These cytokines are optionally used in combination.

A person skilled in the art would recognize that other immune modulatory molecules, including molecules that promote APC function are suitable for use in constructs of the present disclosure.

### d) Delivery Vectors

It will be appreciated by one skilled in the art that a variety of delivery vectors and expression vehicles are usefully employed to introduce a modified DNA molecule into a cell. Vectors that are useful comprise lentiviruses, oncoretroviruses, expression plasmids, adenovirus, and adeno-associated virus. Other delivery vectors that are useful comprise herpes simplex viruses, transposons, vaccinia viruses, human papilloma virus, Simian immunodeficiency viruses, HTLV, human foamy virus and variants thereof. Further vectors that are useful comprise spumaviruses, mammalian type B retroviruses, mammalian type C retroviruses, avian type C retroviruses, mammalian type D retroviruses, HTLV/BLV type retroviruses, and lentiviruses.

Vectors such as those listed above have been employed to introduce DNA molecules into cells for use in gene therapy. Examples of vectors used to express DNA in cells include: Kanazawa T, Mizukami H, Okada T, Hanazono Y, Kume A, Nishino H, Takeuchi K, Kitamura K, Ichimura K, Ozawa K. Suicide gene therapy using AAV-HSVtk/ganciclovir in combination with irradiation results in regression of human head and neck cancer xenografts in nude mice. Gene Ther. 2003 January; 10(1):51-8. Fukui T, Hayashi Y, Kagami H, Yamamoto N, Fukuhara H, Tohnai I, Ueda M, Mizuno M, Yoshida J Suicide gene therapy for human oral squamous cell carcinoma cell lines with adeno-associated virus vector. Oral Oncol. 2001 April; 37(3):211-5.

i. Retroviral Vectors

The safety facet of suicide gene therapy relies on efficient delivery and stable, consistent expression of both the therapeutic and the safety component genes. LVs, a subset of retroviruses, transduce a wide range of dividing and non-dividing cell types with high efficiency, conferring stable, long-term expression of the transgene²⁵⁻²⁷.

The vector is optionally a lentiviral vector that has a pHR′ backbone and comprises 5′-Long terminal repeat (LTR), HIV signal sequence, HIV Psi signal 5′-splice site (SD), delta-GAG element, Rev Responsive Element (RRE), 3′-splice site (SA), Elongation factor (EF) 1-alpha promoter and 3′-Self inactivating LTR (SIN-LTR). Optionally, one makes vectors with the CMV promoter. The lentiviral vector optionally comprises a central polypurine tract (cPPT; SEQ ID NO: 2) and a woodchuck hepatitis virus post-transcriptional regulatory element (WPRE; SEQ ID NO: 3),

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, incapsidation, and expression, in which the sequences to be expressed are inserted.

In one embodiment the Lentigen lentiviral vector described in Lu, X. et al. Journal of gene medicine (2004) 6:963-973 is used to express the DNA molecules.

In one embodiment the application describes a lentiviral vector expressing a TAA cassette and/or an immune modulatory cassette molecule. In one embodiment the lentiviral vector comprises a 5′-Long terminal repeat (LTR), HIV signal sequence, HIV Psi signal 5′-splice site (SD), delta-GAG element, Rev Responsive Element (RRE), 3′-splice site (SA), Elongation factor (EF) 1-alpha promoter and 3′-Self inactivating LTR (SIN-LTR). It will be readily apparent to one skilled in the art that optionally one or more of these regions is substituted with another region performing a similar function.

Gene treatment requires the transgene product to be expressed at sufficiently high levels. Transgene expression is driven by a promoter sequence. The polymerase drives transcription of the transgene.

Enhancer elements can be used to increase expression of modified DNA molecules or increase the lentiviral integration efficiency. In one embodiment the lentiviral vector further comprises a nef sequence. In a preferred embodiment the lentiviral further comprises a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. In an alternate preferred embodiment, the lentiviral vector further comprises a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to lentiviral vector results in a substantial improvement in the level of transgene expression from several different promoters, both in vitro and in vivo. In a further preferred embodiment, the lentiviral vector comprises both a cPPT sequence and WPRE sequence. In one embodiment the construct comprises a TAA cassette and optionally a lysosomal targeting cassette operatively linked to the TAA cassette. In one embodiment the TAA cassette is a polynucleotide encoding a CEA protein, variant or fragment thereof. In another embodiment, the TAA cassette is a polynucleotide encoding a HER/neu protein, variant or fragment thereof. In another embodiment the construct comprises an immune modulatory cassette. In one embodiment the immune modulatory cassette is a polynucleotide that encodes IL-12. In a further embodiment, the construct comprises a TAA cassette and an immune modulatory cassette and optionally comprises a lysosomal targeting sequence cassette. In yet a further embodiment the TAA cassette is a polynucleotide encoding a CEA protein, variant or fragment thereof or HER-2/neu protein, variant or fragment thereof, the immune modulatory cassette is a polynucleotide encoding IL-12 and the lysosomal targeting sequence is derived from alpha-galactosidase A.

The vector also comprises in an alternate embodiment an internal ribosome entry site (IRES) sequence that permits the expression of multiple polypeptides from a single promoter. Accordingly in one embodiment the construct comprises a TAA cassette and optionally a lysosomal targeting cassette operatively linked to the TAA cassette, and an immune modulatory cassette incorporated into pHR′-cppt-EF-IRES-W-SIN. In one embodiment the TAA cassette comprises a polynucleotide that encodes for CEA. In another embodiment the TAA cassette comprises a polynucleotide that encodes HER/neu. The TAA cassette optionally encodes a full length TAA, a fragment or variant thereof. In certain embodiments the immune modulatory cassette is a polynucleotide that encodes IL-12 subunits p35 and p40 and or an IL-12 fusion such as the fusion cDNA obtainable from Invitrogen.

In another embodiment the lentiviral vector comprises a detection cassette. A detection cassette as used herein means a polynucleotide that encodes a protein that is expressed, that is preferably a cell surface protein that is optionally useful for detecting transduced cells, isolating transduced cells by methods such as flow cytometry or clearing transduced cells by targeting transduced cells with an immunotoxin recognizing the targeting/detection cassette encoded protein. In another embodiment, the detection cassette comprises a CD19 molecule or fragment thereof. In another preferred embodiment the construct comprises a targeting polynucleotide incorporated into pHR′-cppt-EF-IRES-W-SIN, pHR′-cppt-EF-huCEA-IRES-hCD19-W-SIN or pHR′-cppt-EF-HER/neuIRES-hCD19-W-SIN. Additionally it will be readily apparent to one skilled in the art that optionally one or more of these elements can be added or substituted with other regions performing similar functions.

In addition to IRES sequences, other elements which permit expression of multiple polypeptides are useful. In one embodiment the vector comprises multiple promoters that permit expression more than one polypeptide. In another embodiment the vector comprises a protein cleavage site that allows expression of more than one polypeptide. Examples of protein cleavage sites that allow expression of more than one polypeptide comprise those listed in the following articles which are incorporated by reference: Retroviral vector-mediated expression of HoxB4 in hematopoietic cells using a novel coexpression strategy. Klump H, Schiedlmeier B, Vogt B, Ryan M, Ostertag W, Baum C. Gene Ther. 200; 8(10):811-7; A picornaviral 2A-like sequence-based tricistronic vector allowing for high-level therapeutic gene expression coupled to a dual-reporter system Mark J. Osborn, Angela Panoskaltsis-Mortari, Ron T. McElmurry, Scott K. Bell, Dario A. A. Vignali, Martin D. Ryan, Andrew C. Wilber, R. Scott Mclvor, Jakub Tolar and Bruce R. Blazar. Molecular Therapy 2005; 12 (3), 569-574; Development of 2A peptide-based strategies in the design of multicistronic vectors. Szymczak A L, Vignali D A. Expert Opin Biol Ther. 2005; 5(5):627-38; Correction of multi-gene deficiency in vivo using a single ‘self-cleaving’ 2A peptide-based retroviral vector. Szymczak A L, Workman C J, Wang Y, Vignali K M, Dilioglou S, Vanin E F, Vignali D A. Nat Biotechnol. 2004; 22(5):589-94. It will be readily apparent to one skilled in the art that other elements that permit expression of multiple polypeptides which identified in the future are useful and may be utilized in the vectors of the disclosure.

ii. Viral Regulatory Elements

The viral regulatory elements are components of vehicles used to introduce nucleic acid molecules into a host cell. The viral regulatory elements are optionally retroviral regulatory elements. For example, the viral regulatory elements may be the LTR and gag sequences from HSC1 or MSCV. The retroviral regulatory elements may be from lentiviruses or they may be heterologous sequences identified from other genomic regions.

One skilled in the art would also appreciate that as other viral regulatory elements are identified, these may be used with the nucleic acid molecules of the disclosure.

### e) Detection Cassette

In certain embodiments, the vector construct comprises a detection cassette. The term “detection cassette” as used herein refers to a polynucleotide that encodes a molecule that is useful for enriching, sorting, tracking and/or killing cells wherein it is expressed. The detection cassette encodes a polypeptide that can be used to detect and/or isolate transduced or transfected cells. The detection cassette is optionally used to determine the efficiency of cell transduction or transfection.

In one embodiment, the detection cassette encodes a polypeptide that protects from a selection drug such as neomycin phosphotransferase or G418. In another embodiment, the detection cassette encodes a fluorescent protein such as GFP. In a further embodiment, the detection cassette is a cell surface marker such as human CD24, murine HSA, human CD25 (huCD25), a truncated form of LNGFR, and truncated CD34.

### f) Safety Components

i. The Cell Surface Protein—Immunotoxin for Killing Transduced Cells

In certain embodiments, the detection cassette is a cell surface protein (marker), such as CD19, CD20 HSA, truncated LNGFR, CD34, CD24 or CD25 which is delivered into target cells. The cell surface expression allows for selective clearance of these cells in vitro and in vivo by administering an immunotoxin (antibody conjugated to a toxin) directed against the cell surface protein. In certain embodiments the detection cassette polypeptide is substantially overexpressed in transduced cells such that these cells are preferentially targeted.

Immunotoxins are described in this application and known in the art, for example, in US patent application no. 20070059275.

Many immunotoxins are approved for use in humans. In one embodiment the immunotoxin is a murine anti-Tac (AT) monoclonal antibodyl9 fused to saporin (SAP)¹⁰⁰ a toxin that irreversibly damages ribosomes by cleaving adenine molecules from ribosomal RNA.21 The inventors have demonstrated both in vitro and in vivo that the AT-SAP (ATS) complex specifically target and kill retrovirally transduced cells that express huCD25. Use of immunotoxins to kill transduced cells are described in CA application Vector Encoding Therapeutic Polypeptide and Safety Elements to Clear Transduced Cells, filed Mar. 27, 2007 which is incorporated herein by reference.

ii. Activator Polynucleotides

Other safety components that can be introduced into the constructs of the disclosure are described in U.S. application Ser. No. 11/559,757, THYMIDYLATE KINASE MUTANTS AND USES THEREOF which is incorporated herein by reference.

In one embodiment, the lentiviral construct further comprises an activator polynucleotide encoding a polypeptide that converts a prodrug to a drug, optionally a modified tmpk polynucleotide. In one embodiment, the activator polynucleotide comprises a tmpk polynucleotide with at least 70%, 70-80%, 80-90%, 90-95%, 95-99%, or 99-99.9%-sequence identity to a modified tmpk polynucleotide, optionally a se

### g) Cassette Variants and Analogs

In the context of a polypeptide, the term “analog” as used herein includes any polypeptide having an amino acid residue sequence substantially identical to any of the wild type polypeptides expressed by the cassettes described herein, for example the TAA cassette for example, CEA, in which one or more residues have been conservatively substituted with a functionally similar residue and which displays the ability of the wildtype molecule for example in the context of CEA, displays a CEA antigenic response similar to wild-type CEA.

Examples of conservative substitutions include the substitution of one non-polar (hydrophobic) residue such as alanine, isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. The phrase “conservative substitution” also includes the use of a chemically derivatized residue in place of a non-derivatized residue provided that such polypeptide displays the requisite activity.

In the context of a polypeptide, the term “derivative” as used herein refers to a polypeptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids. For examples: 4-hydroxyproline may be substituted for proline; 5 hydroxylysine may be substituted for lysine; 3-methylhistidine may be substituted for histidine; homoserine may be substituted for serine; and ornithine may be substituted for lysine. Polypeptides of the present disclosure also include any polypeptide having one or more additions and/or deletions or residues relative to the wild type sequence, so long as the requisite activity is maintained.

The methods of making recombinant proteins are well known in the art and are also described herein.

The nucleic acids described herein can also comprise nucleotide analogs that may be better suited as therapeutic or experimental reagents. The nucleic acid can also contain groups such as reporter groups, a group for improving the pharmacokinetic properties of a nucleic acid.

The nucleic acid molecules may be constructed using chemical synthesis and enzymatic ligation reactions using procedures known in the art. The nucleic acid molecules of the disclosure or a fragment thereof, may be chemically synthesized using naturally occurring nucleotides or variously modified nucleotides designed to increase the biological stability of the molecules.

### h) Virus

The retroviral and lentiviral constructs are in one embodiment, packaged into viral particles. Methods for preparing virus are known in the art and described herein.

Methods of isolating virus are also known in the art and further described herein.

### III. COMPOSITIONS

Another aspect, relates to compositions, including pharmaceutical compositions. The pharmaceutical compositions of this disclosure used in one embodiment to induce or enhance an immune response. In another embodiment, the compositions described herein are used to treat patients having diseases, disorders or abnormal physical states could include an acceptable carrier, auxiliary or excipient.

The pharmaceutical compositions are optionally administered by ex vivo and in vivo methods such as electroporation, DNA microinjection, liposome DNA delivery, and virus vectors that have RNA or DNA genomes including retrovirus vectors, lentivirus vectors, Adenovirus vectors and Adeno-associated virus (AAV) vectors, Semliki Forest Virus. Derivatives or hybrids of these vectors are also useful.

Dosages to be administered depend on patient needs, on the desired effect and on the chosen route of administration. The expression cassettes are optionally introduced into the cells or their precursors using ex vivo or in vivo delivery vehicles such as liposomes or DNA or RNA virus vectors. They are also optionally introduced into these cells using physical techniques such as microinjection or chemical methods such as coprecipitation.

The pharmaceutical compositions are typically prepared by known methods for the preparation of pharmaceutically acceptable compositions which are administered to patients, and such that an effective quantity of the nucleic acid molecule is combined in a mixture with a pharmaceutically acceptable vehicle. Suitable vehicles are described, for example in Remington's Pharmaceutical Sciences (Remington's Pharmaceutical Sciences, Mack Publishing Company, Easton, Pa., USA).

On this basis, the pharmaceutical compositions could include an active compound or substance, such as a nucleic acid molecule, in association with one or more pharmaceutically acceptable vehicles or diluents, and contained in buffered solutions with a suitable pH and isoosmotic with the physiological fluids. The methods of combining the expression cassettes with the vehicles or combining them with diluents is well known to those skilled in the art. The composition could include a targeting agent for the transport of the active compound to specified sites within cells.

### IV. METHODS AND USES

i. Expressing TAA in Cells

In another aspect, the application describes methods of expressing a TAA in a cell, in one embodiment in an APC cell such as a DC cell.

In one embodiment, the method comprises contacting a cell with a composition, vector construct or virus described herein.

The TAA polynucleotide may be incorporated into an appropriate expression vector which ensures good expression of the TAA and/or the cassettes described herein. For example, vectors described herein are suitable.

Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule and regulatory sequences selected on the basis of the host cells to be used for expression, which is operatively linked to the nucleic acid molecule. Operatively linked or operably linked is intended to mean that the nucleic acid is linked to regulatory sequences in a manner which allows expression of the nucleic acid.

The application therefore includes a recombinant expression vector containing a nucleic acid molecule disclosed herein, or a fragment thereof, and the necessary regulatory sequences for the transcription and translation of the inserted protein-sequence.

Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press, San Diego, Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

Recombinant expression vectors can be introduced into host cells to produce a transformed host cell. The terms “transformed with”, “transfected with”, “transformation” “transduced” and “transfection” are intended to encompass introduction of nucleic acid (e.g. a vector or vector construct) into a cell by one of many possible techniques known in the art. The phrase “under suitable conditions that permit transduction or transfection of the cell” refers to for example for ex vivo culture conditions, such as selecting an appropriate medium, agent concentrations and contact time lengths which are suitable for transfecting or transducing the particular host. Suitable conditions are known in the art and/or described herein. The term “transformed host cell” or “transduced host cell” as used herein is intended to also include cells capable of glycosylation that have been transformed with a recombinant expression vector disclosed herein. Prokaryotic cells can be transformed with nucleic acid by, for example, electroporation or calcium-chloride mediated transformation. For example, nucleic acid can be introduced into mammalian cells via conventional techniques such as calcium phosphate or calcium chloride co-precipitation, DEAE-dextran mediated transfection, lipofectin, electroporation or microinjection. Suitable methods for transforming and transfecting host cells can be found in Sambrook et al. (Molecular Cloning: A Laboratory Manual, 3rd Edition, Cold Spring Harbor Laboratory Press, 2001), and other laboratory textbooks. Suitable methods for transducing cells are known in the art and are also described herein.

In one embodiment, vector constructs are introduced into cells that are used for transplant or introduced directly in vivo in mammals, preferably a human. The vector constructs are typically introduced into cells ex vivo using methods known in the art. Methods for introducing vector constructs comprise transfection, infection, electroporation. These methods optionally employ liposomes or liposome like compounds. Introduction in vivo optionally includes intravenous injection and/or intratumoral injection and/or intranodal injection. These methods are described more fully elsewhere.

In certain embodiments, the cell is contacted with a composition vector construct and/or isolated virus described herein, for example an isolated virus comprising a lentiviral vector and a TAA cassette, under conditions that permit transduction or transfection of the cell. Methods of transducing cells are well known in the art. Methods of transducing cells with lentivirus are also described herein.

In certain embodiments, a cell maturing agent is added. In certain embodiments, TNF α or IL-12 is added to the cell.

In one aspect of the present disclosure, methods for expressing a vector or construct of the disclosure in cells for immunotherapy are provided.

ii. Cell Types for Transduction and Isolated Cells

Antigen presenting cells are particularly useful for transduction with the compositions of the disclosure. APCs, as indicated by their name present antigens to cells and function in eliciting an immune response. APCs presenting tumor associated antigens, either as a result of endogenous mechanisms or exogenous introduction of a polynucleotide encoding a TAA or fragment or variant thereof, promotes an anti-tumor response against tumors expressing the particular antigen presented. Immune modulatory molecules such as IL-12 expressed in the APC presenting the tumor associated antigen aids in enhancing the immune response and optionally the anti-tumor effect.

In particular dendritic antigen presenting cells are preferably transduced. Dendritic Cells (DCs) are the most potent antigen presenting cells. They are derived from primitive CD34⁺ hematopoietic cells. DCs have a ‘veiled’ morphology and elicit B cell and T cell immune responses, especially from quiescent effector cells. DCs express MHC genes and accessory co-stimulatory/adhesive molecules, and can migrate to T cell areas of lymphoid tissue to recruit and incite immune effector cells. Lastly, DCs have a major advantage over other hematopoietic cells for development of clinical gene therapy. That advantage is the fact that DC-based leukemias have not been reported. Thus DCs are inherently resistant to transformation events. The transduced DC cells are useful as a DC vaccine.

Other APCs include B cells and mesenchymal stem cells and engineered artificial APC based on 293 or 3T3 cells or the like. The APC are in a preferred embodiment, autologous dendritic cells. In certain embodiments, the DC cells are autologous DC, or derived and/or or propagated from autologous DC.

Cell types that are useful in one embodiment of the present disclosure include, but are not limited to, antigen presenting cells, particularly DC cells, stem cells (both embryonic and of later ontogeny), cord blood cells, and immune cells such as T cells, bone marrow cells and peripheral blood mononuclear cells. T-cells are optionally CD4 positive, CD8 positive or double positive. In addition, DC and T cells are optionally mature T cells. In one embodiment DC cells are transduced with a vector construct or virus of the disclosure, isolated and administered to a host. In another embodiment the DC cells are mature DC cells. In an alternate embodiment stem cells are transduced, isolated and administered to a host.

Methods for isolating DC and other APC are known in the art and are further described herein.

Cell lines are optionally transduced. For example human T cell leukemia Jurkat T cells, human erythro-leukemic K562 cells, human prostate cell lines DU145 and PC3 cells are optionally transduced or transfected with polynucleotides of the disclosure.

Compositions and vector constructs described herein are usefully introduced into any cell type ex vivo. The compositions and vector constructs described herein may also be introduced into any cell type in vivo.

Accordingly, the disclosure also provides in one aspect a cell (including for example an isolated cell in vitro, a cell in vivo, or a cell treated ex vivo and returned to an in vivo site) expressing a TAA cassette, for example a CEA. In one embodiment, the cell is transduced with a composition, vector construct or virus described herein.

iii. Methods of Isolating Cells

After transduction or transfection with vectors comprising elements such as the TAA, targeting polynucleotide, cells expressing these molecules are optionally isolated by a variety of means known in the art. In certain embodiments, the cells are isolated by cell sorting or flow cytometry using an antibody to the targeting/detection cassette encoded selection marker. Additionally cell sorting is useful to isolate modified cells where the targeting cassette is a fluorescent protein such as EGFP.

Cells expressing polynucleotides of the disclosure are, in an alternate embodiment, isolated using magnetic sorting. Additionally, cells may be isolated by drug selection. In one embodiment, a vector comprising a drug resistance gene and a polynucleotides of the disclosure is introduced into cells. Examples of drug resistance genes include, but are not limited to, neomycin resistance gene, blasticidin resistance gene (Bsr), hygromycin resistance gene (Hph), puromycin resistance gene (Pac), Zeocin resistance gene (Sh ble), FHT, bleomycin resistance gene and ampicillin resistance gene. After transduction or transfection, modified cells including the drug resistance gene are selected by adding the drug that is inactivated by the drug resistance gene. Cells expressing the drug resistance gene survive while non-transfected or non-transduced cells are killed. A person skilled in the art would be familiar with the methods and reagents required to isolate cells expressing the desired polynucleotides.

In one embodiment cells are isolated from the transduction or transfection medium and/or the viral preparation. For example the cells may be spun down and/or washed with a buffered saline solution. Accordingly, the cells can comprise a population of cells comprising transduced and untransduced cells. In certain embodiments, the population of cells comprises at least 1%, 2-5%, 5-10%, 10-15%, 15-20%, 20-25%, 25-30%, 30-40%, 40-50%, 50-60%, 60-70%, 70-80%, 80-90%, 90-95%, 95-99% or more than 99% vector construct transduced or transfected cells.

ii. Inducing/Enhancing an Immune Response

In another aspect, the application describes methods of inducing or enhancing an immune response in a subject comprising administering a composition, vector construct, isolated virus, isolated or transduced cell, and/or population of cells described herein. In one embodiment, the application provides a method of inducing or enhancing a memory immune response in a subject in need thereof comprising administering a composition, vector construct, isolated virus, isolated or transduced cell, and/or population of cells described herein.

In another embodiment, the application provides use of a composition, vector construct, isolated viruses, isolated or transduced cells and/or population of cells described herein for inducing or enhancing an immune response.

In another embodiment, the application provides use of a composition, vector construct, isolated viruses, isolated or transduced cells and/or population of cells described herein for the manufacture of a medicament for inducing or enhancing an immune response in a subject.

In one embodiment, the immune response induced or enhanced comprises a CD4+ mediated immune response.

In one embodiment, transduced cells, a population of cells and/or a composition comprising said cells are administered to a subject. In another embodiment, the cells, population of cells and/or composition are administered with an adjuvant. For example, LPS, KLH, CpG, GM-CSF, Montanide ISA-51, or QS21, etc. In addition, the cells, population of cells and/or composition is administered once, or repeated. For example, the cells and or population of cells are administered a second time to boost the immune response.

In certain embodiments, the cells are introduced by intravenous injection, IP injection, subcutaneously or intradermally. Other suitable methods are described elsewhere.

In one embodiment, dendritic cells are obtained from a subject, and genetically modified to express a TAA. The transduced cells or population of cells comprising transduced cells is irradiated and administered to the subject. Accordingly in certain embodiments, clinical use of the modified cells is restricted to the subject from whom the dendritic cell was derived.

In another embodiment, the application provides use of dendritic cells genetically modified to express a TAA, wherein the cells have been irradiated, for inducing or enhancing an immune response in a subject.

In another embodiment, the application provides use of dendretic cells genetically modified to express a TAA, wherein the cells have been irradiated, for the manufacture of a medicament for inducing or enhancing an immune response in a subject.

iii. Treatment

The methods and uses of the disclosure are useful for treating a variety of cancers. For example, vector constructs comprising CEA are useful for treating colon cancer, metastatic colon cancer, rectal, stomache, pancreatic, breast and/or non-small cell lung cancer or any cancer where CEA is expressed and/or other cancers overexpressing CEA.

Cancers such as breast and prostate are amenable to the DC-mediated therapy as demonstrated by vaccination with the murine HER2/neu. Melanomas also have well characterized TAAs that would be amenable to immunotherapy using LVs expressing these TAAs or antigenic fragments thereof. A person skilled in the art would recognize that tumors with characterized tumor antigens such as gastric, pancreatic, lung, ovarian, etc. are amenable treatment with the methods of the disclosure.

The inventors have used murine models of cancer to demonstrate that TAA expressing antigen presenting cells such as dendritic cells are useful as vaccines for the therapeutic and prophylactic treatment of tumors expressing the TAA molecule.

In certain embodiments combinations of different transduced cells are used or administered to a subject in need. For example a DC transduced with lenti-huCEA is used or administered in combination with DC transduced with lenti-IL-12. Alternatively, DC transduced with lenti-lysosomal targeting cassette-huCEA-IL-12 is used or administered in combination with tumor cell transduced with lenti-IL-12.

Compositions of the disclosure are in some embodiments directly administered to a subject. Compositions comprising a TAA cassette wherein the TAA cassette is a polynucleotide that encodes CEA polypeptide or a fragment or variant thereof is in one embodiment administered to a subject with a CEA expressing cancer. In one embodiment the CEA expressing cancer is colon cancer Similarly, a construct comprising a HER-2/neu TAA cassette is administered to a subject with a HER-2/neu expressing tumor. Combination of compositions of the disclosure are optionally combined and administered to a subject in need thereof. The application also provides use of a composition described herein comprising for example a vector construct comprising a TAA cassette for treating TAA expressing tumor. In another embodiment the application provides use of a composition described herein for the manufacture of a medicament for treating the TAA expressing cancer.

The methods disclosed herein are useful for inducing and enhancing an immune response in a subject. In one embodiment, the subject has cancer. In another embodiment, the subject is in remission. In a further embodiment, the subject has an increased risk of cancer.

Vectors containing the nucleic acid molecules of the disclosure are typically administered to mammals, preferably humans, in gene therapy using techniques described below. The polypeptides produced from the nucleic acid molecules are also optionally administered to mammals, preferably humans. The disclosure relates to a method of medical treatment of a mammal in need thereof, preferably a human, by administering to the mammal a vector of the disclosure or a cell containing a vector construct of the disclosure.

The disclosure includes compositions uses and methods for providing a coding nucleic acid molecule to a subject such that expression of the molecule in the cells provides the biological activity of the polypeptide encoded by the coding nucleic acid molecule to those cells. A coding nucleic acid as used herein means a nucleic acid that comprises nucleotides which specify the amino acid sequence, or a portion thereof, of the corresponding protein. A coding sequence may comprise a start codon and/or a termination sequence.

The disclosure includes methods, uses and compositions for providing a coding nucleic acid molecule to the cells of an individual such that expression of the coding nucleic acid molecule in the cells provides the biological activity or phenotype of the polypeptide encoded by the coding nucleic acid molecule. The method also relates to a method for providing a subject having a disease, disorder or abnormal physical state with a biologically active polypeptide by administering a nucleic acid molecule of the present disclosure. The method may be performed ex vivo or in vivo. Gene therapy methods and compositions are demonstrated, for example, in U.S. Pat. Nos. 5,869,040, 5,639,642, 5,928,214, 5,911,983, 5,830,880, 5,910,488, 5,854,019, 5,672,344, 5,645,829, 5,741,486, 5,656,465, 5,547,932, 5,529,774, 5,436,146, 5,399,346 and 5,670,488, 5,240,846. The amount of polypeptide will vary with the subject's needs. The optimal dosage of vector may be readily determined using empirical techniques, for example by escalating doses (see U.S. Pat. No. 5,910,488 for an example of escalating doses).

In one embodiment, Vector constructs are introduced into cells that are used for transplant or introduced directly in vivo in mammals, preferably a human. The vectors are typically introduced into cells ex vivo using methods known in the art. Methods for introducing vectors comprise transfection, infection, electroporation. These methods optionally employ liposomes or liposome like compounds.

The method also relates to a method for producing a stock of recombinant virus by producing virus suitable for gene therapy comprising modified DNA encoding a gene of interest. This method preferably involves transfecting cells permissive for virus replication (the virus containing therapeutic gene) and collecting the virus produced.

Cotransfection (DNA and marker on separate molecules) may be employed (see eg U.S. Pat. No. 5,928,914 and U.S. Pat. No. 5,817,492). As well, a detection cassette or marker (such as Green Fluorescent Protein marker or a derivative) may be used within the vector itself (preferably a viral vector).

The disclosure includes a method for producing a recombinant host cell capable of expressing a nucleic acid molecule of the disclosure comprising introducing into the host cell a vector of the disclosure.

The disclosure also includes a method for expressing a polypeptide in a host cell of the disclosure including culturing the host cell under conditions suitable for coding nucleic acid molecule expression. The method typically provides the phenotype of the polypeptide to the cell.

In these methods, the host cell is optionally a APC, stem cell, a T cell or a dendritic cell.

Cancers treatable by the methods of the invention include (but are not limited to): colon cancer (any adenocarcinoma arising from the large intestine); rectal cancer (adenocarcinoma of the rectum); and adenocarcinomas arising from the stomach, pancreas, small intestine, breast, lung, and prostate. Any of these cancers treatable by the methods of the invention would include the primary (initial cancer) as well as recurrences (cancers that relapse in the same location after any disease-free interval, and metastases (cancers that arise at a distance from the primary via lymphatic, hematologic, or intraperitoneal spread) either at the time of the primary cancer or after a disease-free interval.

### Prostate Cancer

The inventors transduced DCs with an erbB2tr lentiviral construct and determined that about 47% of DCs overexpressed erbB2tr. To shown that low doses of transduced DCs could protect mice from prostate cancer, the inventors performed prime/boost vaccinations with 2×10³ or 2×10⁵ erbB2tr-transduced DCs. Six weeks post-vaccination, mice were simultaneously challenged with the aggressive wild-type RM1 prostate cancer cell line and an erbB2tr-expressing variant (RM1-erbB2tr). Whereas control mice developed both tumors, all recipients of 2×10⁵ erbB2tr-transduced DCs developed only wild-type RM1 tumors. Remarkably, one-third of mice vaccinated with just 2×10³ erbB2tr-transduced DCs also demonstrated erbB2tr-specific tumor protection. Protection against RM1-erbB2tr tumors was associated with sustained levels of anti-erbB2tr antibody production and also correlated with erbB2tr-specific induction of IL-2, IFN-γ, and TNF-α secretion from re-challenged splenocytes. The inventors demonstrate that adoptive transfer of syngeneic DCs engineered to express a self-antigen through efficient lentivirus-based gene transfer activates both cellular and humoral immunity, protecting host animals against specific tumor challenge.

In one embodiment, compositions and vectors of the disclosure are used to treat cancer by adoptive therapy. Adoptive therapy or adoptive (immuno)therapy refers to the passive transfer of immunologically competent tumor-reactive cells into the tumor-bearing host to, directly or indirectly, mediate tumor regression. The feasibility of adoptive (immuno)therapy of cancer is based on two fundamental observations. The first of these observations is that tumor cells express unique antigens that can elicit an immune response within the syngeneic (genetically identical or similar especially with respect to antigens or immunological reactions) host. The other is that the immune rejection of established tumors can be mediated by the adoptive transfer of appropriately sensitized lymphoid cells. Clinical applications include transfer of peripheral blood stem cells following non-myeloablative chemotherapy with or without radiation in patients with lymphomas, leukemias, and solid tumors.

### CEA Positive Cancers-Colon Cancer

As mentioned CEA is a tumor marker whose expression is increased in a variety of cancers, particularly colon cancer. CEA levels can reflect the presence and/or progression of a colon cancer. A non-CEA positive colon cancer can recur as a CEA positive colon cancer.

Accordingly the methods provided herein are useful for subjects with a CEA positive cancer or subjects with an increased risk of developing a CEA positive cancer.

In one aspect of the present disclosure ADC/DC, T cells or stem cells (either embryonic or of later ontogeny) are transduced with compositions vector constructs or virus of the disclosure. Cells expressing these vector constructs are isolated and adoptively transferred to a host in need thereof. In one embodiment the bone marrow of the recipient is T-cell depleted. Methods of adoptive T-cell transfer are known in the art (J Translational Medicine, 2005 3(17): doi;0.1186/1479-5876-3-17, Adoptive T cell therapy: Addressing challenges in cancer immunotherapy. Cassian Yee). This method is used to treat solid tumors and does not require targeting the vector-transduced expressing cells to the tumor since the modified.

### Dosing

The uses and methods provide in certain embodiments, that a composition, transduced cell, population or cells, vector construct or virus described herein is administered to the subject. The compositions, cells, vector constructs and viruses of the present application may be administered at least once a week in one embodiment. However, in another embodiment, the composition, transduced cell, population or cells, or vector construct may be administered to the subject from about one time per week, one time per 14 days, or one time per 28 days. The length of the treatment period depends on a variety of factors, such as the severity of the disease, the age of the patient, the concentration and the activity of the compounds of the present application, or a combination thereof. In one embodiment, the treatment is chronic treatment and the length of treatment is 1-2 weeks, 2-4 weeks or more than 4 weeks. The treatment regimen can include repeated treatment schedules. It will also be appreciated that the effective amount or dosage of the compound used for the treatment or prophylaxis may increase or decrease over the course of a particular treatment or prophylaxis regime. Changes in dosage may result and become apparent by standard diagnostic assays known in the art. In some instances, chronic administration may be required.

The number of cells administered varies with the expression level and/or number of transduced cell or population of cells.

In one embodiment, 0.1-1, 1-5, 5-10, 10-20, 20-30, 30-40, 40-50, 50-60, 60-70, 70-80, 80-90, 90-100 or more than 100, ×10⁶ cells are administered. In other embodiments, the dose of DCs administered is escalated. For example a first dose may consist of 5×10⁶, and additional doses may be escalated to 20×10⁶ cells. A person skilled in the art would understand that the dose can be a single administration or divided.

### Combination Treatments

In certain embodiments, the vector constructs, transduced cells, population of cells and or compositions comprising these, are administered in combination with other therapies. For example, the vector constructs, transduced cells, population of cells, virus and or compositions comprising these may be administered before or after chemotherapy suitable for the cancer being treated. In other embodiments wherein the cancer is a solid cancer, the vector constructs, transduced cells, population of cells and or compositions comprising these are administered before or after surgery.

The compositions, vector constructs, virus and cells for inducing or enhancing an immune response or treating a subject in need thereof may be optionally administered prior to treatment with another therapy, during treatment with another therapy or after treatment with another therapy. For example administration may take place prior to surgery, if indicated, or subsequent to surgery.

In another embodiment, the application provides use of a composition, vector construct, isolated virus or isolated or transduced cell in combination with a second therapeutic intervention.

iv. Killing Transduced Cells

Cells transduced with a composition, vector construct or virus comprising a safety component, are optionally deleted from the host.

Compositions and vector constructs comprising a safety component are usefully introduced into any cell type ex vivo where it is desirable to provide a mechanism for killing the modified cells. In certain embodiments, a prodrug is administered to kill cells that comprise an activator polynucleotide. In one embodiment AZT or similar prodrug is administered to a subject comprising cells modified with a modified tmpk polynucleotide.

For example, in some cases, irradiation may negatively effect the ability of the transduced cells to induce an immune response eg irradiation may cause cell death in certain cell populations. Use of an activator polynucleotide or other mechanism to remove unwanted cells transplanted into the subject is alternatively used in such situations.

Alternatively, in certain embodiments wherein the vector construct comprises a detection cassette that expresses a cell surface protein such as truncated CD19, an immunotoxin is administered.

In certain embodiments, the methods further comprise monitoring cancer progression. Cancer progression can be monitored using known methods.

### VI. RESEARCH TOOLS AND POLYPEPTIDE PRODUCTION

### The CEA Transgenic Mouse

To facilitate murine studies, investigators have developed a transgenic mouse engineered to express human CEA in a tissue-specific manner similar to that seen in humans (Eades-Perner, A. M. et al., 1994; Zhou, H. et al., 2004). Use of these mice permits preclinical evaluation of immune tolerance using vectors designed for eventual clinical utility and better approximates the CEA expression patterns in colorectal cancer patients. Lastly, crossing these CEA-transgenic animals with mice that spontaneously develop tumours of the GI tract (such as the APC^{Min/+} mice) has resulted in offspring that develop spontaneous colorectal tumours expressing high levels of human CEA DCs (Kenneth, W. H. et al., 2005). There are several mouse models expressing human CEA that are of great interest to evaluate CEA-based cancer vaccines (16).

### Polypeptide Production and Research Tools

A cell line (either an immortalized cell culture or a stem cell culture) transfected or transduced with a polynucleotide of the disclosure (or variants) is useful as a research tool to measure levels of expression of the coding nucleic acid molecule and the activity of the polypeptide encoded by the coding nucleic acid molecule.

Another aspect of the disclosure is an isolated polypeptide produced from a nucleic acid molecule or vector of the disclosure according to a method of the disclosure.

### EXAMPLES

### Example 1

### Constructs

A fusion cDNA of IL-12 is cloned downstream of an IRES sequence in Lenti-huCEA to make a construct that expresses CEA and IL-12.

A lysosome targeting sequence is fused to CEA maintaining the correct reading frame of translated CEA. The polynucleotide of the fused lysosome targeting sequence and CEA molecule is subcloned in a lentiviral vector.

A lentiviral construct is made by subcloning the lysosome targeting sequence fused to CEA downstream of a promoter and further subcloning IL-12 downstream of an IRES sequence.

A virus preparation is prepared according to methods described elsewhere it known in the art.

### Example 2

### Lenti-huCEA Vaccination

### Murine DC Generation and Transduction

DCs were generated according to Lutz et al. (1999) [37] with slight modifications. Briefly, bone marrow was flushed from femurs and tibiae of C57BL/6 mice using a 25G needle. Red blood cells (RBCs) were lysed using RBC Lysing Buffer (Sigma, St. Louis, Mo.). Remaining cells were plated in 10-cm petri dishes at a concentration of 2×10⁵ cells/ml in a total volume of 10 ml/dish. DC media consisted of RPMI with 10% FBS (PAA Laboratories, Etobicoke, ON), 1% penicillin/streptomycin, 5×10⁻⁵ M 2-mercaptoethanol (both from Sigma), 40 ng/ml rmGM-CSF and 5 ng/ml rmIL-4 (both from Peprotech, Rocky Hill, N.J.). Cells were infected on day 3 of culture with either LV/CEA or LV/enGFP, or left uninfected. Half-volume media changes were done every other day starting on day 4. On day 7, 50 ng/ml of rmTNF-α (Peprotech) was added for 24-48 hrs of DC maturation.

### Flow Cytometric Analysis of DCs and Tumor Cells

DCs and tumor cells were stained with an anti-erbB2 primary antibody (Ab4, Oncogene Science, Tarzana, Calif.) and a PE-conjugated poly-adsorption goat anti-mouse Ig secondary antibody (BD Biosciences Canada, Mississauga, ON) and cell surface expression of CEA was measured using a FACS Calibur (BD). For phenotypic analysis of DCs, the following BD antibodies were used with appropriate isotype controls: PE- or FITC-conjugated anti-CD11c (clone HL3), purified anti-CD80 (clone 1G10), and FITC-conjugated anti-CD86 (clone GL1), FITC-conjugated anti-1-A^{b} (clone AF6-120.1).

### Allogeneic Mixed Lymphocyte Reaction

Transduced and control DCs were harvested on day 9 of culture and dosed with 30 cGy in a Gammacell 3000 Elan ¹³⁷Co irradiator (Nordion International Inc., Ottawa, Canada). Freshly isolated splenocytes from C57BL/6 and BALB/c mice were B cell-depleted using goat anti-mouse Ig magnetic beads (Dynal, Brown Deer, Wis.). The remaining T cell-enriched population was plated in triplicate in a 96-well U-bottom plate (BD) at 2×10⁵ cells per well in T cell media. Next, serially-diluted irradiated DCs (range of 0 to 0.6×10⁵ cells/well) were added. Following 4 days of co-incubation, 1 uCi of [³H]methyl-thymidine was added to each well for 20 hrs. Thymidine incorporation was measured using a Beckman LS 1801 Liquid Scintillation Counter (Beckman).

Transduction of Dendritic Cells with Lenti Hu-CEA

Dendritic cells were isolated from human PBSC by adherence on a cell culture plate and cultured in presence of IL-4 and GmCSF. On day 2, DCs were transduced by the lenti-huCEA virus with an MOI of 10. On day 5, TNFα was added for maturation.

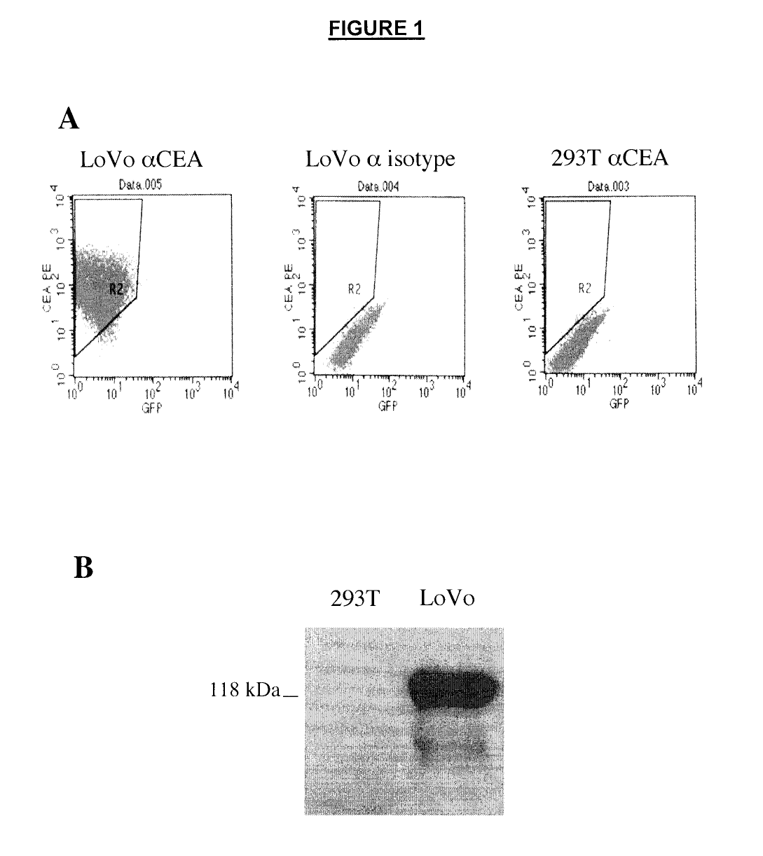

The ability of an antibody to recognize CEA was first tested. FIG. 1 illustrates that BD Pharmingen CEA antibody specifically recognizes CEA expressed by colon cancer line LoVo cells. No band is detected in 293T which do not express CEA.

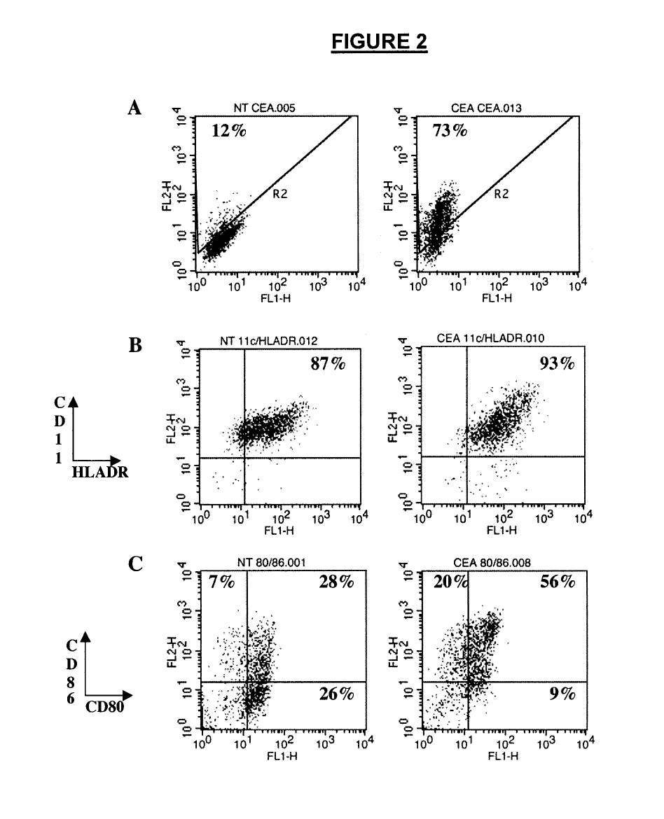

FIG. 2 plots the expression of DC markers on day 7 determined by flow analyses FIG. 2A shows the induction of CEA expression by the transduction (right) compared to the basal level of CEA on NT cells (left).

FIG. 2B shows that DCs markers expression is not affected.

FIG. 2C shows a higher CD86 expression in transduced cells.

### Immunizations and Tumor Inoculations

C57BL/6 mice are injected i.p with 2×10⁵ or 2×10³ DCs transduced with LV huCEA LV/enGFP, or non-transduced controls in 200 ml of PBS. As a positive control, one group of 5 mice is injected with 2×10⁵ CEA-transduced DCs along with CFA (Sigma). These immunizations are repeated 2 weeks later. Six weeks after the second immunization, 6 of 10 mice in each cohort are challenged with bilateral tumors and the remaining mice were sacrificed for splenocyte cytokine secretion analyses. For the tumor challenge, each mouse is injected s.c. with 2×10⁵ CEA expressing cells (in 200 ml of PBS) in the dorsal left and right flanks, respectively. Starting six days later, the length (I), width (w), and height (h) of each tumor is measured by caliper on a daily basis. Tumor volume is calculated by multiplying l×w×h.

### Assay for Antibody Production Against CEA

Antibody titres reactive with CEA are assessed after tumor challenge.

### Example 3

After construction and sequencing of the vector, recombinant virions are produced by triple transfections of 293T cells. These virions are readily pseudotyped by VSV-g (as above) or other viral glycoproteins. Virions are concentrated by ultracentrifugation and used to first infect target cells such as naïve 293T cells. This allows determination of a functional viral titer. Next primitive murine hematopoietic cells that can be differentiated into effective antigen presenting cells, especially DCs, are infected. Both mouse and human DCs are infected. Infection frequencies are determined functionally by Western blots and flow cytometric analyses for human CEA expression; as well the cell surface profile of the transduced cell population is ascertained.

Next, in vitro assays are performed to evaluate T cell responses initiated by the transduced DCs. Appropriate human tumor lines serve as targets for CTL responses. Murine tumor lines are established that express human CEA, by gene transfer techniques. Studies such as cytokine release assays and allogeneic MLRs are again performed as described in Medin J A, Liang S B, Hou J W, Kelley L S, Peace D J, Fowler D H. Efficient transfer of PSA and PSMA cDNAs into DCs generates antibody and T cell antitumor responses in vivo. Cancer Gene Ther. 2005 June; 12(6):540-51 herein incorporated by reference.

In vivo experiments are performed in mice. These involve murine models directly along with adoptive experiments in immune deficient animals using human cells. Immune responses are tracked and tumor measurements established under a number of conditions. Human CEA-transgenic mouse (available from Dr. John Thompson Institute of Immunobiology, Univ. of Freiburg) are employed. Pre-clinical data is obtained from the expression of heterologous human CEA in normal mice as described for human prostate antigens (Medin J A, Liang S B, Hou J W, Kelley L S, Peace D J, Fowler D H. Efficient transfer of PSA and PSMA cDNAs into DCs generates antibody and T cell antitumor responses in vivo. Cancer Gene Ther. 2005 June; 12(6):540-51. herein incorporated by reference).

CEA transgenic strains are crossed with a human HLA-A2Kb strain (Eades-Perner, A. M. et al., 1994). These animals express human CEA and the α₁ and α₂ domains of human HLA-A2.1 which is the HLA variant that is most common in North America. Progeny are vaccinated with lenti hu-CEA transduced DC. Adaptive vaccinations are conducted in immune deficient animals (NOD/SCID animals pretreated with an antibody against murine NK cells to improve engraftment) allowing xenotransplants in another parallel context. Lastly, we also pursue the addition of secondary genes to bicistronic recombinant lentiviral constructs that augment anti-tumor responses initiated by these strategies. We use the cDNA for murine IL-12 and murine RANKL. The former affects T cell responses and the latter has the potential to extends the lifespan of DCs in an autocrine fashion.

Use of these VSV-g pseudotyped vectors readily infects human cells. Here we do these experiments in tandem with the murine experiments using bone marrow-derived DCs obtained from normal donors and from colon cancer patients. We do infections and measure in vitro responses in our true target cell population. Assessments of outcomes of these experiments (and adoptive ones using human cells in the murine xenotransplant model mentioned above) are facilitated by human antigen specific tetramers for CEA antigens that have been developed by Beckman.

### Example 4

### Materials and Methods

### Lentiviral Vector (LV) Construction and Preparation of High Titer Stocks

The enhanced green fluorescent protein (enGFP)-containing LV pHR′EF-GW-SIN (LV/enGFP) was described previously [34]. To construct an erbB2tr-containing recombinant LV (LV/erb), the enGFP cDNA sequence was excised from pHR′EF-GW-SIN by EcoRI (New England Biolabs, Beverly, Mass.) digestion and replaced with the cDNA sequence for erbB2tr (GI:28386210). This erbB2tr sequence was amplified by PCR from the Invitrogen pYX-Asc plasmid (IMAGE 5702040) with Taq polymerase (both Invitrogen, Burlington, ON), ligated into PCR-Script Amp(+) SK(+) (Stratagene, La Jolla, Calif.), and excised by EcoRI digestion.

LV particles were generated by calcium-phosphate transfection of 293T cells (kindly provided by Dr. Michele Calos, Stanford University, Calif.) with the plasmids pCMVDR8.91, pMD.G [35], and either LV/enGFP or LV/erb. Viral supernatants were collected at 24 and 48 hrs post-transfection, filtered using a 0.45 mm filter, and concentrated at 19,000×g for 2 hrs using an Optima L-100 XP Ultracentrifuge (Beckman Coulter Canada Inc., Mississauga, ON). Concentrated virus preparations were serially diluted and titered on 293T cells by FACS analysis as previously described [15].

### Mice and Cell Lines

C57BL/6 (Jackson Laboratories, Bar Harbor, Me.) and BALB/c (Charles River, Wilmington, Mass.) mice were bred and housed under specific pathogen-free conditions at the UHN Animal Resource Centre. RM1 cells, a murine prostate cancer cell line syngeneic to the C57BL/6 strain, were kindly provided by Dr. Timothy Thomson (Baylor). The clonal RM1-erbB2tr cell line was generated by transducing RM1 cells to overexpress a kinase-truncated form of erbB2 (erbB2tr) and then isolating single cell clones. For these transductions, an onco-retroviral pUMFG-erbB2tr vector was constructed (Mossoba and Medin, unpublished) and transfected into the E86 packaging cell line to generate virus-producing E86 cells, as previously described [36]. In vitro growth characteristics of RM1-erbB2tr vs. WT RM1 cells were nearly identical (data not shown). All animal experiments were performed under a protocol approved by the Animal Care Committee at the UHN.

### Murine DC Generation and Transduction

DCs were generated according to Lutz et al. (1999) [37] with slight modifications. Briefly, bone marrow was flushed from femurs and tibiae of C57BL/6 mice using a 25G needle. Red blood cells (RBCs) were lysed using RBC Lysing Buffer (Sigma, St. Louis, Mo.). Remaining cells were plated in 10-cm petri dishes at a concentration of 2×10⁵ cells/ml in a total volume of 10 ml/dish. DC media consisted of RPMI with 10% FBS (PAA Laboratories, Etobicoke, ON), 1% penicillin/streptomycin, 5×10⁻⁵ M 2-mercaptoethanol (both from Sigma), 40 ng/ml rmGM-CSF and 5 ng/ml rmIL-4 (both from Peprotech, Rocky Hill, N.J.). Cells were infected on day 3 of culture with either LV/erb or LV/enGFP, or left uninfected. Half-volume media changes were done every other day starting on day 4. On day 7, 50 ng/ml of rmTNF-α (Peprotech) was added for 24-48 hrs of DC maturation.

### Flow Cytometric Analysis of DCs and Tumor Cells

DCs and tumor cells were stained with an anti-erbB2 primary antibody (Ab4, Oncogene Science, Tarzana, Calif.) and a PE-conjugated poly-adsorption goat anti-mouse Ig secondary antibody (BD Biosciences Canada, Mississauga, ON) and cell surface expression of erbB2tr was measured using a FACS Calibur (BD). For phenotypic analysis of DCs, the following BD antibodies were used with appropriate isotype controls: PE- or FITC-conjugated anti-CD11c (clone HL3), purified anti-CD80 (clone 1G10), and FITC-conjugated anti-CD86 (clone GL1), FITC-conjugated anti-1-A^{b} (clone AF6-120.1).

### Allogeneic Mixed Lymphocyte Reaction

Transduced and control DCs were harvested on day 9 of culture and dosed with 30 cGy in a Gammacell 3000 Elan ¹³⁷Co irradiator (Nordion International Inc., Ottawa, Canada). Freshly isolated splenocytes from C57BL/6 and BALB/c mice were B cell-depleted using goat anti-mouse Ig magnetic beads (Dynal, Brown Deer, Wis.). The remaining T cell-enriched population was plated in triplicate in a 96-well U-bottom plate (BD) at 2×10⁵ cells per well in T cell media. Next, serially-diluted irradiated DCs (range of 0 to 0.6×10⁵ cells/well) were added. Following 4 days of co-incubation, 1 uCi of [³H]methyl-thymidine was added to each well for 20 hrs. Thymidine incorporation was measured using a Beckman LS 1801 Liquid Scintillation Counter (Beckman).

### Immunizations and Tumor Inoculations

C57BL/6 mice were injected i.p with 2×10⁵ or 2×10³ DCs transduced with LV/erbB2tr, LV/enGFP, or non-transduced controls in 200 ml of PBS. As a positive control, one group of 5 mice was injected with 2×10⁵ erbB2tr-transduced DCs along with CFA (Sigma). These immunizations were repeated 2 weeks later. Six weeks after the second immunization, 6 of 10 mice in each cohort were challenged with bilateral tumors and the remaining mice were sacrificed for splenocyte cytokine secretion analyses (see below). For the tumor challenge, each mouse was injected s.c. with 2×10⁵ RM1-NT and RM1-erbB2tr cells (in 200 ml of PBS) in the dorsal left and right flanks, respectively. Starting six days later, the length (I), width (w), and height (h) of each tumor was measured by caliper on a daily basis. Tumor volume was calculated by multiplying l×w×h.

Measurement of Anti-erbB2tr Antibody from Mouse Plasma

Approximately 200 ul of blood was collected weekly from the tail vein of each mouse into EDTA-coated tubes (Sarstedt, Montreal, Canada). Plasma was isolated by centrifugation at 18,000×g at 4° C. for 20 min. Plasma anti-erbB2 measurements were performed using a novel flow cytometry-based ELISA method the inventors developed that was based on that described by Piechocki et al. (2002) [38]. Briefly, RM1-erbB2tr and wild-type (WT) RM1 cells were first stained with diluted plasma samples or primary Ab4 antibody (above) for 1 hr on ice followed by 2 washes with PBS. Secondary staining with PE-conjugated poly-adsorption goat anti-mouse antibody was done for 1 hr on ice, again followed by 2 PBS washes. 7-AAD was added to each sample to exclude dead cells from flow cytometric analysis. The mean fluorescence intensity (MFI) value in the FL2 channel was measured on a FACS Calibur for each sample. A standard curve was generated by plotting Ab4 antibody concentration versus the MFI values of the Ab4-stained RM1-erbB2tr cells. This curve was used to convert MFI values of plasma anti-erbB2 levels from each mouse cohort into antibody concentration values. Each experiment was performed three times and the SD of the means was calculated.

### Cytokine Secretion Assays

Spleens from immunized and naïve control C57BL/6 mice were dissociated into single-cell suspensions and treated with RBC lysis buffer. RBC-depleted splenocytes were cryopreserved in freezing medium (90% FCS, 10% DMSO), then thawed when needed using a method described by Maecker et al. (2005) [39]. Briefly, cryovials were warmed to 37° C. in a waterbath and the contents diluted dropwise with an equal volume of warm media. Diluted cells were transferred to a 50 ml tube containing 8 ml of warm media per cryovial of added cells and centrifuged at 290×g for 7 min. Cell pellets were resuspended and brought to a final concentration of 5×10⁶ cells/ml in RPMI medium containing 10% FCS, 1% penicillin/streptomycin, 1% minimal essential amino acids (Invitrogen), and 5×10⁻⁵ M 2-mercaptoethanol. Next, 200 ml of cell suspensions were transferred to each well of 96-well round-bottom plates (BD) and incubated at 37° C. for 18 hrs. Splenocytes were then collected from each well, counted, and plated in 24-well plates at 3×10⁶ cells per well in 1 ml. Approximately 2×10⁵ freshly prepared DCs that were left non-transduced or that were transduced with LV/erb, LV/enGFP were added to each well. Co-cultures were incubated at 37° C. for 24 hrs and supernatants were collected and stored at −20° C. IFN-γ, IL-2, TNF-α, IL-4, and IL-10 levels were measured from thawed supernatant samples by Bio-Plex multiplex sandwich immunoassays (Bio-Rad Laboratories, Hercules, Calif.).

### Statistical Analysis

Student's t tests were used to perform pairwise comparisons. Differences in means were considered statistical significance at P<0.05.

Immunity was generated towards the self-antigen erbB2 in mice using DCs that were genetically engineered to express erbB2tr. The inventors showed that vaccinating mice with lentivirally transduced DCs could impart long-term erbB2-specific immunity and protection against subsequent challenge with erbB2-expressing tumors. In this model the inventors used an aggressive RM1 prostate tumor cell line that the inventors have modified to express erbB2tr. The inventors chose to focus on low-dose vaccination strategies. This provides a low-dose DC immunotherapy strategy using LVs as gene transfer tools engineering expression of target TAAs.

### Results:

1. DCs are Efficiently Transduced with Lentivirus

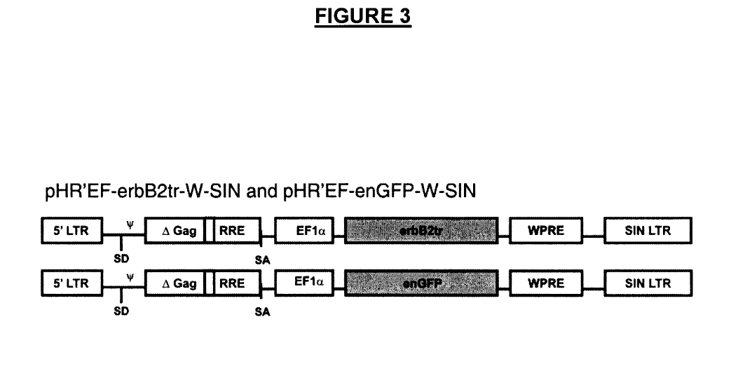

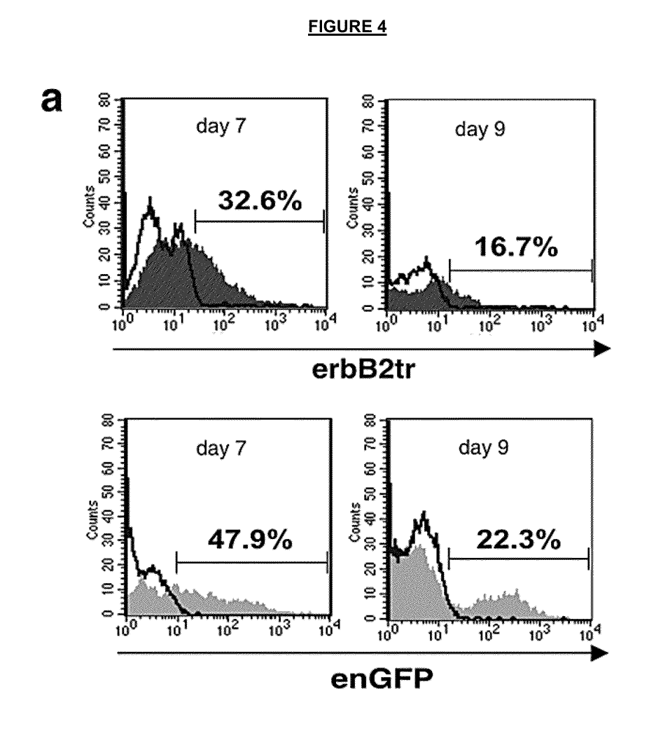

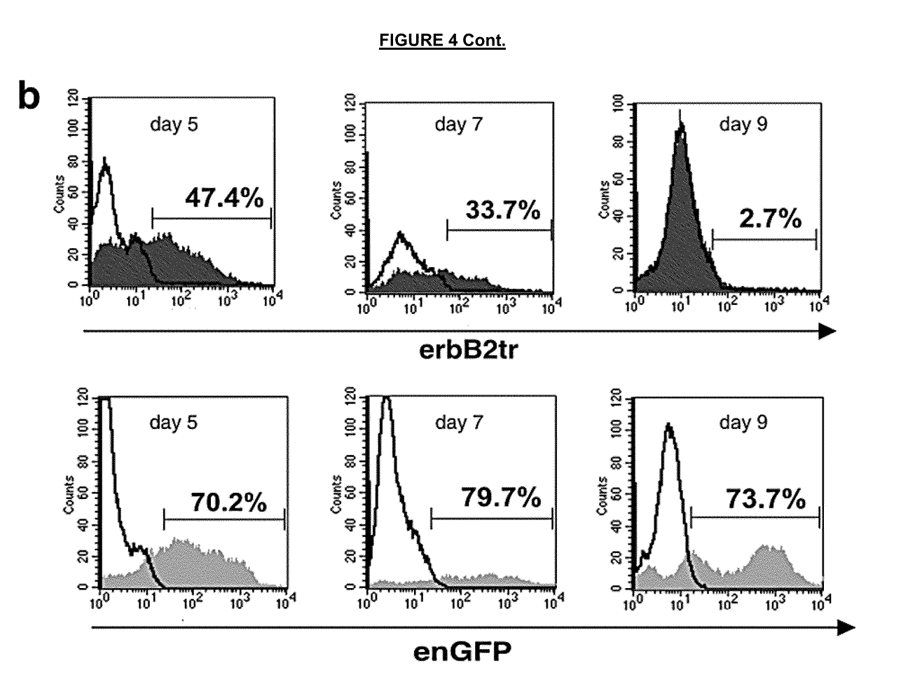

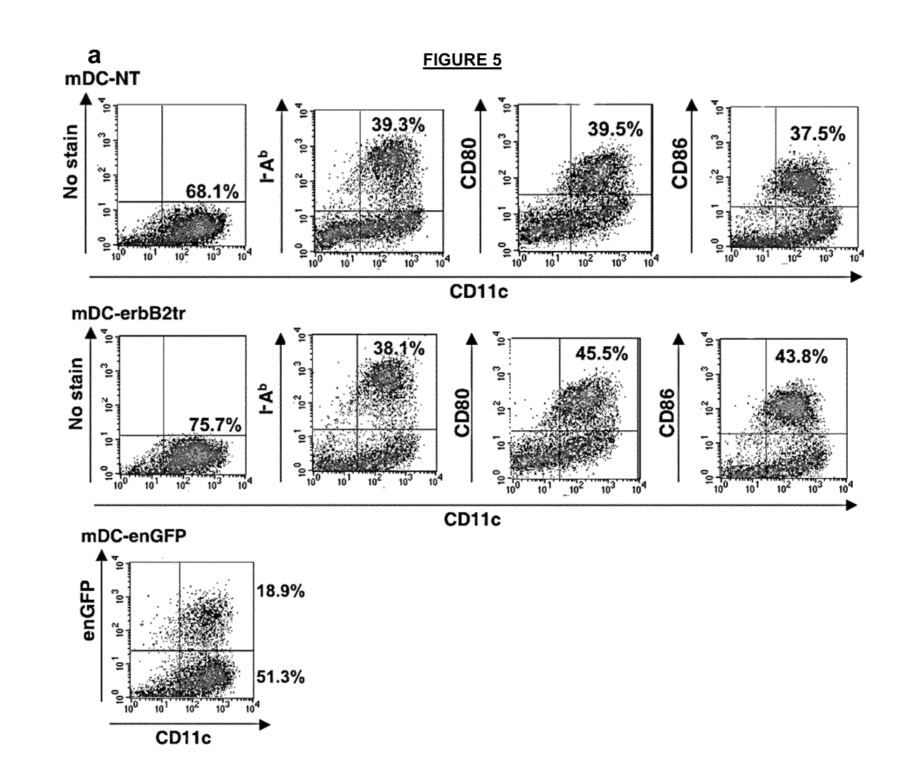

A lentiviral transfer vector encoding erbB2tr, a truncated (kinase-deficient) form of the murine self-antigen erbB2 (LV/erb) was constructed (FIG. 3); an enGFP LV was previously described [15]. Titers of produced LVs usually approximated between 5×10⁶ and 3.6×10⁸ functional infectious viral particles per ml. To determine the transduction efficiency of LV/erb, the inventors infected BM-derived murine DCs on day 3 of in vitro culture. In an initial pilot experiment, the inventors determined that between 20% and 70% of a DC population was productively infected after one overnight incubation with LV/erb. Next, the inventors initiated a large-scale in vivo experiment designed to test the efficacy of prime/boost vaccinations with LV-transduced DCs in mediating erbB2tr-specific anti-tumor immunity. Freshly-derived DCs were transduced and their expression levels of erbB2tr and enGFP were monitored over time. On culture day 7 for DCs used in the first immunization, the inventors observed that 32.6% of transduced DCs were erbB2tr⁺ and 47.9% were enGFP⁺, respectively (FIG. 4a). By day 9, when DCs were injected, erbB2tr⁺ and enGFP⁺ cells had decreased to 16.7% and 22.3%, respectively. For the second immunization, the inventors also checked expression levels at day 6 and found that 47.4% of DCs were erbB2tr⁺ and 70.2% were enGFP⁺ (FIG. 4b) at that time. The percentage of erbB2tr⁺ DCs decreased steadily to 33.7% on day 7 and 2.7% on day 9. The percentage of enGFP⁺ DCs was 79.7% and 73.7% on days 7 and 9, respectively.

2. Lentiviral Transduction does not Alter DC Phenotype or Allostimulatory Capacity

To determine whether transducing DCs with the inventors recombinant LVs at reasonable MOIs led to changes in phenotype, the inventors first performed flow cytometry to compare the expression of typical surface molecules on mature transduced and control DCs. The inventors assessed the percentage of cells expressing the myeloid marker CD11c, MHC II molecule I-Ab, along with co-stimulatory molecules CD80 and CD86. DCs used for the first scheduled vaccinations expressed similar levels of CD11c; 68.1% of the non-transduced DC cultures were CD11c⁺ compared to 75.7% and 70.2% for erbB2tr- and enGFP-transduced DC cultures, respectively (FIG. 5a). Further comparisons revealed that the percentage of CD11c⁺ I-Ab⁺ DCs was nearly identical between non-transduced and LV/erb-transduced DCs (39.3% vs. 38.1%, respectively). A minor difference in the percentage of CD11c⁺ CD80⁺ DCs was measured from non-transduced compared to erbB2tr-transduced cultures (39.5% vs. 45.5%, respectively). Similarly, 37.5% of non-transduced DCs and 43.8% of erbB2tr-transduced DCs were CD11c⁺ CD86⁺ (FIG. 5a).

The DCs generated for the second vaccination exhibited similar trends (FIG. 5b). The percentage of CD11c⁺ cells in the control cultures was 91.1%, compared to 90.3% for erbB2tr-transduced DCs, and 79.8% for enGFP-transduced DCs. The percentages of CD11c⁺ I-Ab⁺ were similar for control and erbB2tr-transduced DCs (60.0% vs. 67.0%, respectively). Comparing the percentages of DCs expressing costimulatory molecules, the inventors found that 52.3% of non-transduced DCs and 59.0% or erbB2tr-transduced DCs were CD11c⁺ CD80⁺. A minor difference in the CD11c⁺ CD86⁺ percentage was also detected between the control and transduced DCs (62.3% vs. 65.0%, respectively).

To determine whether transduction with LV/enGFP or LV/erb affected DC function, the inventors compared the ability of non-transduced and transduced DCs to induce an allogeneic mixed lymphocyte reaction (MLR). The inventors cultured H-2^{b}-expressing DCs (transduced and control) with either H-2^{d} splenocytes from BALB/c mice or H-2^{b} splenocytes from C57BL/6 mice and measured splenocyte proliferation by thymidine incorporation (FIGS. 5c,d). No significant differences were found between the allostimulatory capacities of non-transduced DCs and either LV/enGFP- or LV/erb-transduced DCs.

Vaccination with Low Doses of LV-Modified DCs Generates Antigen-Specific Tumor Protection

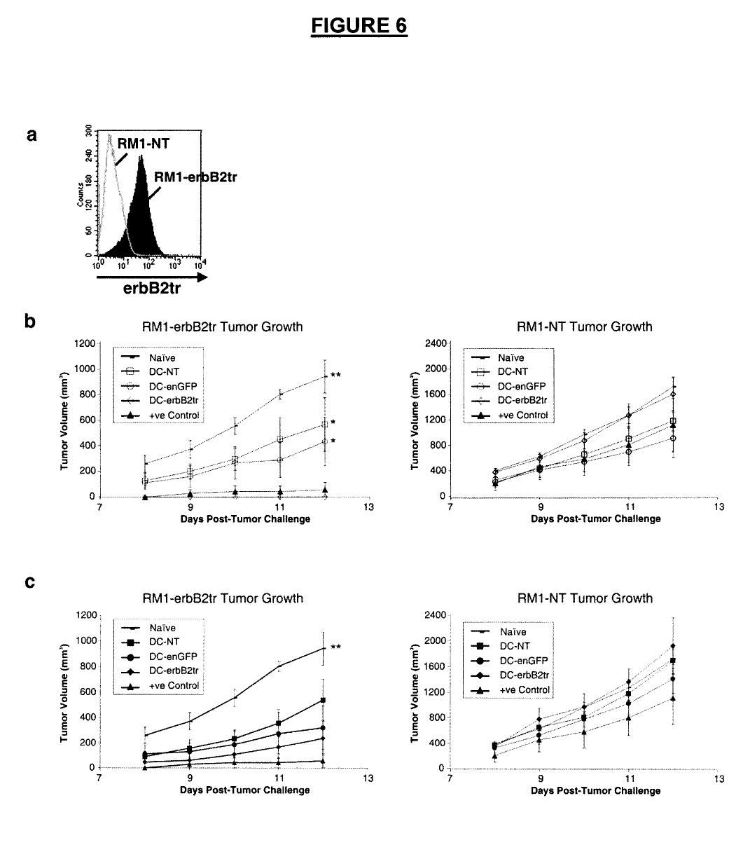

Although many studies employing DC-vaccination strategies [16] evaluate tumor protection around 1-2 weeks post-vaccination, the inventors chose to investigate the long-term benefits of a prime/boost vaccination strategy by challenging mice ectopically with RM1 prostate tumors 6 weeks after the second vaccination. RM1 cells grow aggressively in vivo, providing a stringent model for assessing tumor growth after vaccination; subcutaneously implanting just 10⁴ RM1 cells will yield palpable tumors within 1 week and can compromise mouse survival by 10 days post-implantation [22]. The RM1 tumor cell line lacks endogenous erbB2 expression according to the FACS analysis (FIG. 6a). Therefore, the inventors generated a clonal cell population of erbB2tr-expressing RM1 cells (RM1-erbB2tr) by onco-retroviral transduction followed by clonal isolation (FIG. 6a).

In a pilot study, the inventors first tested the efficacy of three immunizations using doses of 2×10⁵ and 2×10³ of erbB2tr-transduced DCs to protect against subsequent challenge with erbB2-expressing tumors. The inventors vaccinated mice three times with either erbB2tr-transduced or non-transduced DCs. Two weeks after the third vaccination, the inventors injected mice with non-transduced RM1 cells (RM1-NT) on one dorsal flank and RM1-erbB2tr cells on the opposite dorsal flank in order to generate a bilateral tumor model in the same individual. Whereas many tumor protection studies utilizing virally-transduced DCs typically inject between 0.5×10⁶ and 1×10⁶ DCs per immunization [16], the focus of this pilot study was to investigate the possible benefits of using markedly lower doses of transduced DCs. In that study, the inventors observed that both erbB2 immunization regimens offered considerable protection against erbB2tr-expressing RM1 tumors specifically, compared to that obtained using non-transduced DCs (data not shown).

To examine these low-dose outcomes in more detail, the inventors next performed a larger study. The inventors again used the dose of 2×10⁵ DCs for immunizing one cohort of mice, and the 100-fold lower dose of 2×10³ DCs for another, however the inventors reduced the number of vaccinations per animal to just two. The inventors injected mice twice with either control, erbB2-transduced, or enGFP-transduced DCs, two weeks apart. The inventors next challenged animals with the same tumor challenge. As a positive control, one group of mice was immunized twice with 2×10⁵ erbB2tr-transduced DCs mixed with the Complete Freund's Adjuvant (CFA) emulsion. Using this prime/boost strategy, none of the 6 mice that were immunized with 2×10⁵ erbB2tr-transduced DCs showed RM1-erbB2tr tumor growth, whereas RM1-NT tumors grew rapidly (FIG. 6b). In contrast, the control naïve mice and mice immunized with 2×10⁵ non-transduced or enGFP-transduced DCs developed both RM1-NT and RM1-erbB2tr tumors with an aggressive growth profile that necessitated euthanasia within 2 weeks. Strikingly, significant tumor protection from RM1-erbB2tr tumors was also observed in mice that were immunized with the 100-fold lower dose of 2×10³ erbB2-transduced DCs (FIG. 6c). In this group, 2 of 6 mice displayed complete tumor protection until the point of sacrifice at 2 weeks post tumor challenge, and 2 of 6 mice showed reduced RM1-erbB2tr growth compared to control cohorts.

Mice Vaccinated with DC-erbB2tr Show Strong Antigen-Specific Humoral Immunity

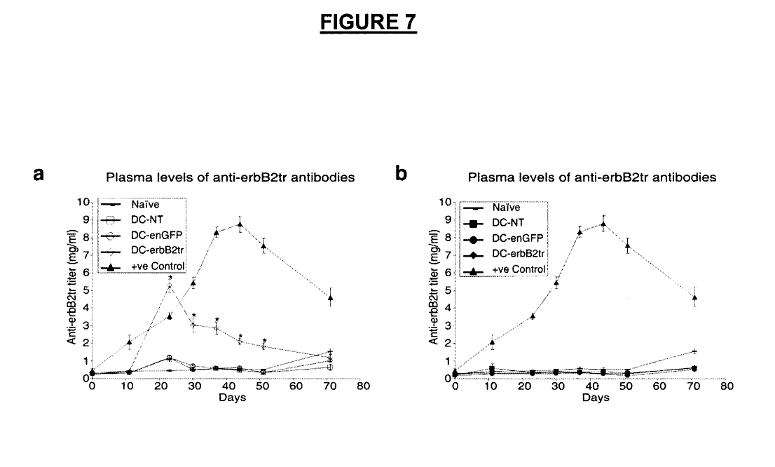

To show mechanisms by which DC-erbB2tr immunizations break tolerance against erbB2tr, the inventors collected blood from each mouse on a weekly basis and measured the plasma levels of anti-erbB2 antibodies. As shown in FIG. 7a, mice immunized with CFA+ erbB2tr-transduced DCs (positive control) began producing modest levels of anti-erbB2tr antibodies. Following the second vaccination, these mice showed steadily increasing titers that peaked at about 45 days after the first DC injection. Relatively high antibody levels were detected up to 70 days post-prime vaccination when the mice were sacrificed. In our experimental groups, mice injected twice with 2×10⁵ erbB2tr-transduced DCs showed a rapid increase in antibody titer after the boost vaccination. Indeed, within 10 days, the average anti-erbB2tr titer rose to over 5 times the average level in control mice. After this peak, a steady decline was measured, but specific anti-erbB2tr antibodies were still detectable at day 50, when mice were inoculated with tumors. In the non-vaccinated group of mice, challenge with RM1-erbB2 tumors caused a slight increase in antibody titers, revealing the weak immunogenicity of these tumors. Injecting the lower dose of 2×10³ transduced DCs did not lead to detectable anti-erbB2tr antibodies at least within the sensitivity limits of this assay, despite the anti-tumor effects observed above (FIG. 7b).

3. Analysis of Cytokine Secretion from Splenocytes

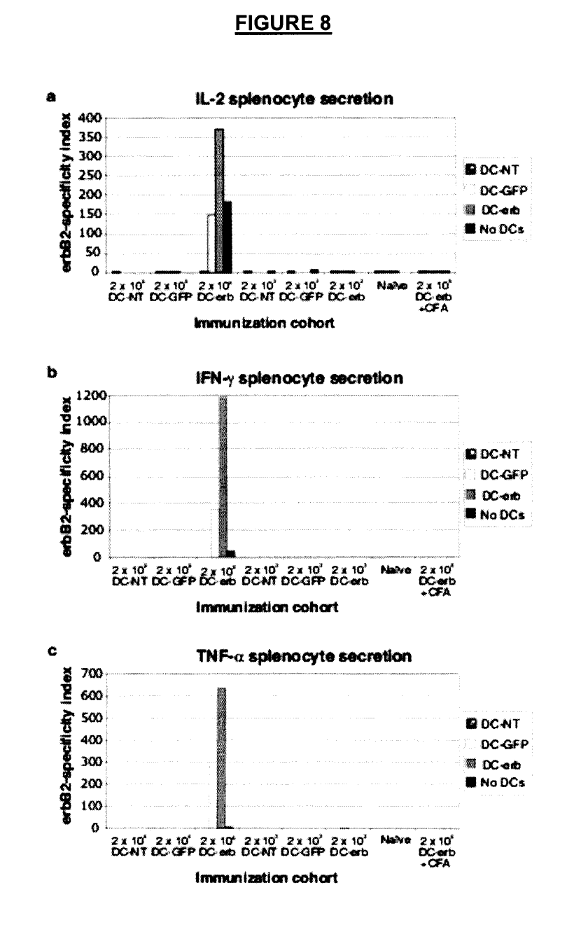

To further evaluate mechanisms, the inventors harvested the spleens from naïve and immunized mice 6 weeks after the second vaccination. The inventors re-stimulated splenocytes in vitro for 24 hrs with freshly prepared transduced or control DCs and analyzed culture supernatants for production of Th1 (IL-2, IFN-γ, and TNF-α) and Th2 (IL-4 and IL-10) cytokines. The inventors found that recipients of 2×10⁵ erbB2tr-transduced DCs produced greater levels of IL-2, IFN-γ, and TNF-α following in vitro re-stimulation with erbB2tr-transduced DCs relative to controls (FIG. 8). In contrast, this erbB2tr-specific cellular response was absent from the supernatants of all other mouse cohorts. To quantify the levels of antigen-specificity, the inventors calculated a ‘specificity index’ by normalizing cytokine concentration results from each group to the values obtained from re-stimulation with non-transduced DCs (FIG. 8). The inventors found that splenocytes taken from mice vaccinated with 2×10⁵ erbB2tr-transduced DCs produced approximately 370-fold more IL-2 after re-stimulation with erbB2tr-transduced DCs than with non-transduced DCs. An even greater specificity index value was calculated for the relative increase in IFN-γ; over 1100-fold more IFN-γ was produced after erbB2tr-specific re-stimulation. The inventors also found a 635-fold increase in TN F-α production following in vitro re-stimulation with erbB2tr-transduced DCs relative to controls. Levels of IL-4 and IL-10 in the co-culture supernatants were generally very low and specificity towards erbB2tr was not observed.

### Discussion

This study is the first to demonstrate the use of lentivirally transduced DCs in an immunogene therapy cancer model targeting the self-antigen erbB2 in mice. The method of engineering DCs to present TAAs may play an important role in the potency of immunotherapy schemas. The use of recombinant viral vectors encoding full-length or large portions of TAAs permits transduced DCs to present a broad repertoire of natural immunogenic tumor antigen peptides in stable MHC complexes. The inventors employed an LV-based system and found that DCs could be even more efficiently transduced to express antigens, without compromising their phenotype or function. This finding is especially important given that the effects of lentiviral transduction of murine DCs with erbB2 has not been previously investigated and the inventors LV/GFP transduction efficiency is high.

Genetically engineering DCs to overexpress human or rat erbB2 does not lead to oncogenic effects in DCs [25-28]. To further decrease the possibility of affecting the target DC population by overexpressing a heterologous signaling molecule, the inventors employed a kinase-deficient version of erbB2 to use with the lentiviral construct. The fact that there have been no reports to date of any DC-derived leukemias, indicates that DCs themselves may not be easily amenable to oncogenesis. In the future, no matter what the gene or target cell population, gene therapy protocols may also incorporate additional safety mechanisms, such as a suicide gene therapy strategy [29].

The inventors used only 2 immunizations, in order to decrease the number of DCs required, and were still able to induce specific immune-mediated anti-tumor responses. Prior approaches involving virally transduced DCs administer between 0.5×10⁶ to 1×10⁶ DCs per immunization [16]. Remarkably, the inventors found that a dose as low as 2×10³ DCs offered partial protection against erbB2tr-expressing tumors, indicating that the minimum effective DC dose falls in the range between the tested doses. In addition, these results were obtained in a relatively long-term setting, as mice were tumor challenged 6 weeks after the last immunization.

The inventors observed a humoral response in vaccinated mice that correlated with tumor protection in the 2×10⁵ DC-dose group. Mice vaccinated with 2×10³ erbB2tr-transduced DCs had comparatively low antibody titers. In addition, long-term Th1 immunity was observed in the 2×10⁵ DC-dose mice. It was not detected in the 2×10³ DC dose cohort despite the finding that 2 of 6 of these mice were protected from developing RM1-erbB2tr tumors and an additional 2 had markedly reduced tumor volumes compared to controls. This may reflect the detection limit of the assays, and may also point to the very sensitive nature of this system. It may be that specific anti-tumor immune responses can be induced with very subtle changes to the immune marker profiles. Another possibility is that these assays may not fully capture the contribution of other mitigating factors, for example NK cell-mediated immunity that could contribute to the anti-tumor responses the inventors observed. In the Th1 assays, the erbB2tr specificity of cytokine production in the 2×10⁵ DC-erbB2tr cohort is clear. The inventors did detect low levels of Th1 reactivity towards enGFP, likely because enGFP is a xeno-antigen and therefore inherently foreign to the mouse species. That the inventors did not observe significant levels of Th2 cytokines at 6 weeks post-vaccination is consistent with the waning of anti-erbB2tr antibody levels over time.

Using a completely syngeneic system is imperative for accurately assessing the potency of TAA-expressing DCs in overcoming endogenous self-tolerance mechanisms. Xenogeneic immune responses resulting from the introduction of non-murine antigens into mice can mask syngeneic immunity, thus confounding effects of the administered DC therapy. The use of a mouse model of cancer where tumors grow orthotopically and express a human or rat TAA of interest is one way to avoid unwanted xenogeneic immunity. Unfortunately, there is currently no such mouse model for prostate cancer that is also transgenic for human HER-2/neu. Nevertheless, by creating the erbB2tr⁺ RM1 prostate cancer cell line variant, the inventors could still generate an in vivo model to accurately test the LV-transduced erbB2tr-expressing DCs. Inoculating each mouse with the wild-type and erbB2tr⁺ RM1 cell lines in opposite flanks also gave us the advantage of incorporating a internal negative control into our model and reducing animal-to-animal variance.

The DC vaccination strategy using erbB2-transduced cells was well tolerated. Although the self-antigen erbB2 is naturally expressed at varying levels throughout the mouse body including the lungs, intestines, and brain [30] manifestations of autoimmune toxicity were not observed in the 10 weeks after the beginning of the vaccination schedule. Other groups have also reported that anti-tumor immunity in mouse models can occur without damaging normal tissues, even when self-antigens are targeted [31-33].

In conclusion, the results show that vaccination using relatively low doses of DCs transduced to express a self-antigen safely and effectively protects mice against tumor development in an antigen-specific manner. Tumor protection was associated with antigen-specific cellular and humoral immunity. The recombinant LV system the inventors utilized served as an efficient gene transfer vehicle, which did not adversely affect DCs. Low dose DC-immunotherapy strategies are useful in clinical situations where patient DCs may be scarce. Such cancer immunotherapy vaccines are particularly applicable before tumors are established and in early stage disease, and reduce the need for more intensive treatments with systemic toxicity such as chemotherapy or radiation therapy.

### Example 6

### Mouse Models of Colon Cancer

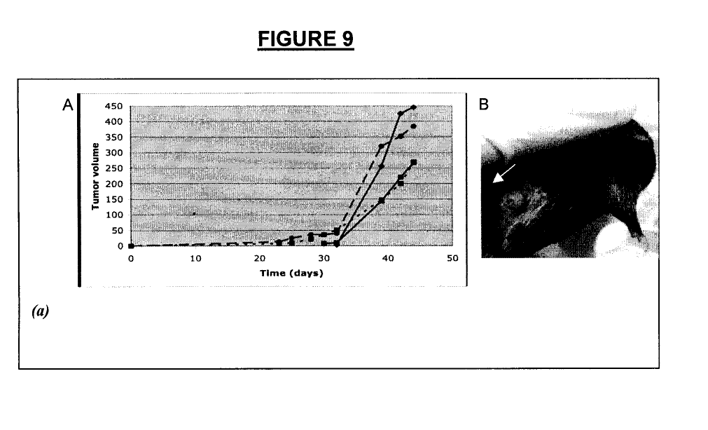

Murine tumor cell lines into were injected in huCEA transgenic mice. Two male heterozygous huCEA transgenic mice (CEA2682) [22], were crossed with wild-type C57BL/6 females. Fifty percent of the pups from this mating were positive for huCEA to establish a colony of huCEA transgenic mice. Tumor growth in these mice was assessed following subcutaneous injection of gastric murine tumorigenic mGC8 or mGC4CEA cells [47] in our animal colony facility (FIG. 15 FIG. 9).

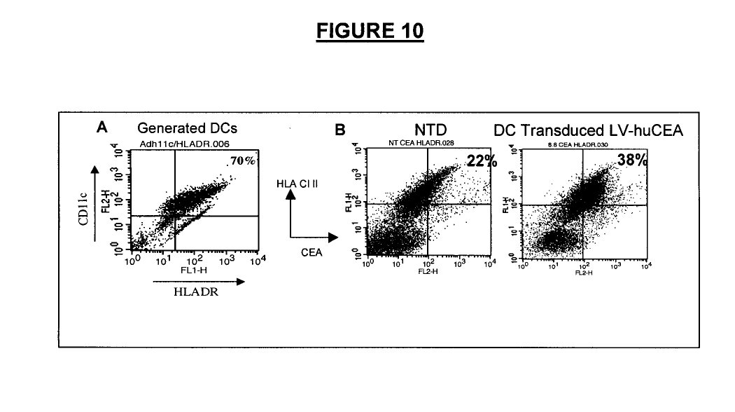

VSV-g pseudotyped vectors are appropriate for clinical applications as they allow infection of human cells. Peripheral blood (PB) derived DCs is obtained from healthy donors and from colon cancer patients, under a REB protocol for this purpose. DCs have been generated from adherent PB cells (FIG. 10, A) and T lymphocytes from non adherent PB cells using appropriate culture conditions known in the art. huDCs have been transduced with the LV/huCEA (FIG. 10). Transduced DCs tranduced with LV/huCEA virus are tested for their ability to activate autologous T lymphocytes against tumor cells expressing huCEA.

### In Vitro Testing of the CEA Vector

The ability of murine DCs transduced with LV/huCEA to initiate a CTL response in vitro is measured, with gastric murine tumor lines expressing huCEA (mGC4CEA) and control cells (mGC8) [47], as targets. Cytokine release assays for IFNγ and allogeneic MLRs are performed [44] to measure the anti-CEA responses.

### In Vivo Models

These cell lines are ectopically inject in huCEA transgenic mice. After subcutaneous injection of either cell line, palpable tumors form [47]. These mice are used to show the ability of our CEA-transduced DCs to overcome self tolerance. The vaccine is administered by direct skin injection of the LV alone [48, 49]. One group of transgenic mice are injected with LV/huCEA-transduced DCs or the LV/huCEA prior to tumorigenic cells. The protective effect is evaluated against establishment of tumors. In a second group of mice the vaccine is administered after the tumor cells, to show the therapeutic effect against pre-established tumors. In both cases, tumor size is monitored to show the anti-tumor efficacy of our approach to shrink tumor size. Cellular and humoral specific immune responses by CTL assays and anti-CEA antibody titers are also monitored.

### Example 6 References

22. Eades-Perner, A. M., et al. Mice transgenic for the human carcinoembryonic antigen gene maintain its spatiotemporal expression pattern. Cancer Res 54(15), 4169-4176. 1994.
47. Nockel J et al. Characterization of gastric adenocarcinoma cell lines established from CEA424/SV40 T antigen-transgenic mice with or without a human CEA transgene. BMC Cancer. 14;6:57, 2006.
48. Kim J H, Majumder N, Lin H, Watkins S, Falo L D Jr, You Z. Induction of therapeutic antitumor immunity by in vivo administration of a lentiviral vaccine. Hum Gene Ther. 2005 November; 16(11):1255-66.
49. Dullaers M, Van Meirvenne S, Heirman C, Straetman L, Bonehill A, Aerts J L, Thielemans K, Breckpot K. Induction of effective therapeutic antitumor immunity by direct in vivo administration of lentiviral vectors. Gene Ther. 2006 April; 13(7):630-40.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### Example 7

### Materials and Methods

Lentiviruses. The huCEA cDNA was obtained to construct LV-huCEA. LV expressing green fluorescent protein (LV-enGFP) was used as a control. Viral functional titers in transducing units (TU) per ml were determined by infection of 293T cells and subsequent analysis of transgene expression by flow cytometry.

Mice. huCEA transgenic mice (huCEA Tg; Ref. 18) were housed in a pathogen-free environment in the animal facility at the University Health Network and studies were performed under Animal Care Committee approval. 8-10 week old huCEA Tg mice were used for anti-tumor immunity studies. For ethical reasons, mice were euthanized before the tumor diameter exceeded 15 mm.

mGC4CEA Tumor Cell Line.

The murine gastric carcinoma cell line expressing huCEA was established from spontaneously developing tumors of CEA424/SV40Tag C57BL/6 mice (19) and used to establish subcutaneous tumors (FIG. 9). These cells were cultured in RPMI 1640 supplemented with 10% heat inactivated fetal calf serum (FCS “Gold”; PAA Laboratories), 2 mM L-glutamine, 100 U/mL penicillin, 100 μg/mL streptomycin, non-essential amino acids and 1 mM sodium pyruvate (GIBCO/Invitrogen).

Tumor Rejection Experiments.

For therapeutic tumor treatment, mice were grafted subcutaneously at day 0 with 0.8×10⁶ mGC4CEA tumor cells in the flank and subsequently immunized in the footpad on days 14 and 21 with PBS or with 0.15×10⁶ transducing units (TU) of LV-enGFP or with 0.15×10⁶ TU of LV-huCEA. Groups were designated respectively as “PBS”, “enGFP”, and “huCEA”. Tumor growth was evaluated by caliper measurement until 28 days post-tumor challenge. The tumor sizes were calculated using the formula: L×W×H. In the second experiment, one additional group of LV-huCEA vaccinated mice was randomly assigned at the beginning to receive one more dose of 0.1×10⁶ TU of LV-huCEA vaccine at day 28. This group was called “huCEALg” (for long-term) whereas the other huCEA group of this experiment was designated “huCEASh” (for short-term). Tumor growth of the huCEALg group was assessed at least until over 50 days post-tumor challenge.

Detection of Anti-huCEA Antibody Response.

Serum samples were collected from huCEA Tg mice before and during the tumor rejection experiments. Based on a protocol by Cusi et al. (20), 96-well microtiter plates were coated with 1 μg/ml purified huCEA (Chemicon International) and incubated at 4° C. overnight. Wells were washed with PBS-0.05% Tween-20 and blocked with 5% heat inactivated FCS in PBS for 2 h at room temperature. Duplicate 100 μl aliquots of sample (sera diluted 1/40) were allowed to react for 1 h at 37° C. Mouse anti-human CEA mAb, COL-1 (Zymed), was diluted 1/200 and used as a positive control. Following washes with PBS-0.05% Tween-20, 100 μl of a 1/30,000 dilution of goat HRP-labeled anti-mouse IgG (Bio-Rad) was added, and the plate was incubated for 1 h at 37° C. After washes with PBS-0.05% Tween-20, the substrate 3,3′,5,5′-tetramethylbenzine (Sigma-Aldrich) was added to each well and allowed to react at room temperature for 30 min in the dark. The reaction was stopped with 100 μl of 1M H₃PO₄ and plates were analyzed by spectrophotometry at 450 nm. Induction rate of antibodies for individual mice were calculated at each time point relative to the OD measured for the pre-immune serum. Positive sera were considered to be those showing an OD at least 2.2 times the pre-immune value.

Splenocyte Harvest and Culture.

Splenic homogenates from huCEA Tg mice were filtered through a 0.45 μm cell strainer and spun for 5 min at 400 rcf=xg?. Red blood cells were lysed in 1 ml of ammonium chloride. The remaining splenocytes were cultured at a concentration of 2×10⁶/ml in RPMI 1640 supplemented with 10% heat inactivated bovine serum (PAA Laboratories), 2 mM L-glutamine, 100 U/mL penicillin, 100 μg/mL streptomycin, 50 μM beta-mercapto-ethanol, and 3.3 mM N-acetyl-cystein. This medium was supplemented every 48 h with 20 U/ml recombinant human IL-2 (Roche) and 20 ng/ml human IL-7 (Preprotech).

Multiple Cytokine Analysis.

Splenocytes were prepared and cultured as above without any stimulation. Supernatant of culture were harvested after 24 and 48 h and frozen for later analysis. IFN-gamma, IL-2, TNF-alpha, IL-4, and IL-10 levels were measured from thawed supernatant samples by Luminex using Bio-Plex multiplex sandwich immunoassays according to the manufacturers protocol (Bio-Rad Laboratories, Hercules, Calif.). IL-4 and IFN-γ secretion by the cells were also assessed by single separate ELISAs according to the manufacturer's instructions (BD Biosciences).

IFN-Gamma Secretion Assay.

Splenocytes were pooled according to group and co-cultured for 48 h with mGC4CEA cells at a maximum spleen cell to tumor cell ratio of 20:1. Each cell type was also cultured alone to determine constitutive cytokine secretion. Evaluation of IFN-gamma secretion by the cells was performed by ELISA according to the manufacturer's instructions (BD Biosciences).

Tetramer Staining.

To detect huCEA-specific CTLs in huCEA Tg mice after vaccination, pooled splenocytes were stained with APC-conjugated anti-CD8 mAb and PE-conjugated huCEA/H-2Db tetramer specific for EAQNTTYL (SEQ ID NO: 23) immunogenic peptide (iTag, Beckman Coulter). After blocking, immunofluorescence staining was performed following the manufacturer's instructions. Immunofluorescence was compared with the appropriate isotype-matched controls and analyzed with CellQuest software using a FACSCalibur cytometer (BD Biosciences).

Immunofluorescence Staining of Tumor Sections.

At the end of the second tumor rejection experiment, tumors from 3 mice per group were harvested and frozen. Sections (4 μm) were fixed for 15 min at room temperature in a 1:1 methenol:acetone solution. Dry sections were washed with cold PBS and incubated for 1 h with a blocking solution (PBS containing 1% BSA and 0.2% gelatin). Sections were incubated for 45 min with anti-CD4 or anti-CD8 primary antibody (eBioscience) at a 1/50 dilution in PBS containing 0.5% BSA and 0.2% gelatin. After washes with PBS, AlexaFluor188-conjugated secondary anti-IgG antibody (Molecular Probes) was added at a dilution 1/200 for 45 min. Secondary antibody alone was used as background control. DAPI solution was used for nuclei staining. 2-3 pictures per section were analyzed using an Axioskop 2 microscope linked to an AxioCam MRc camera (Zeiss). For each area, the ratios between the positive area fractions obtained with Alexafluor488 and DAPI was evaluated using ImageJ software and designated AD ratios. Average was calculated per mouse and then the background was deduced by subtracting the ratio obtained for the negative control. The result was designated as “absolute AD ratios”.

### Results

LV-huCEA Induces Therapeutic Immunity Leading to Subcutaneous mGC4CEA Tumor Regression in huCEA Tg Mice.

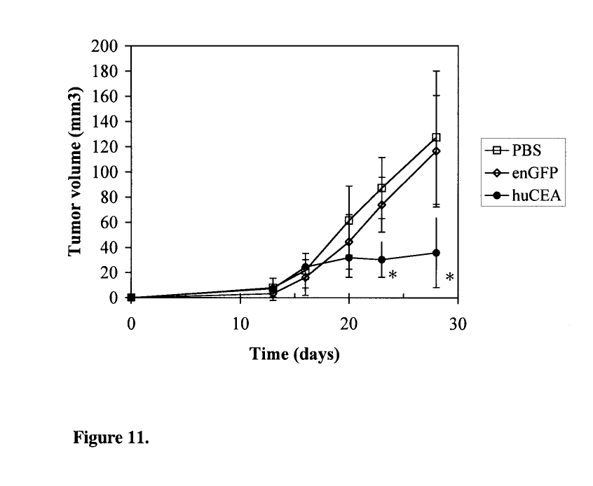

The potential efficacy of LV-huCEA as a vaccine against huCEA-expressing tumors was assessed in vivo in huCEA Tg mice. We evaluated the anti-tumor effect in a relatively stringent model using 14-day established tumors. Mice were injected subcutaneously in the flank with mGC4CEA tumor cells. After the tumors became palpable, mice were vaccinated in the footpad with PBS or LVs engineered to express either huCEA, or enGFP as a negative control. They received a boost injection one week later. Tumor growth was assessed until day 28 in short-term experiments. In duplicate experiments performed according to this schedule, the kinetics of tumor growth in equivalent groups were the same. FIG. 11 shows representative short-term results of the first experiment. We observed stabilization of average tumor sizes in the LV-huCEA vaccinated mice beginning after the first vaccination on day 14. In contrast, tumors in PBS or LV-enGFP vaccinated mice continued to grow at the same rate until the end of the experiment (FIG. 11). Overall, in both experiments, tumors in the LV-huCEA vaccinated mice were significantly smaller than those in the mice of the PBS and enGFP groups (P<0.05 since D23 and P<0.005 at D28). Interestingly, although the average tumor size observed in the huCEA groups was stable over the course of the experiments, we noted different growth rates of individual tumors in those groups: in some mice a tumor regression actually occurred, while tumors were growing slower or at a normal rate in other mice. More than 65% of the LV-huCEA vaccinated mice actually showed a tumor regression both in the first (4/6) and in the second experiment (6/9). This phenomenon was seen a few days after the second injection except in a few mice for which tumor regression began earlier between the two injections (1/4 and 2/6). In each experiment, only one LV-huCEA vaccinated mouse did not respond to the vaccine and showed unrestrained tumor growth. The remaining mice of each LV-huCEA vaccine group showed either reduced tumor growth compared to controls (n=1) or stabilization of tumor size with no measurable growth following vaccination (n=2).

Vaccination with LV-huCEA Breaks Tolerance and Leads to Anti-huCEA Antibody Production in the Sera of huCEA Tg Mice.

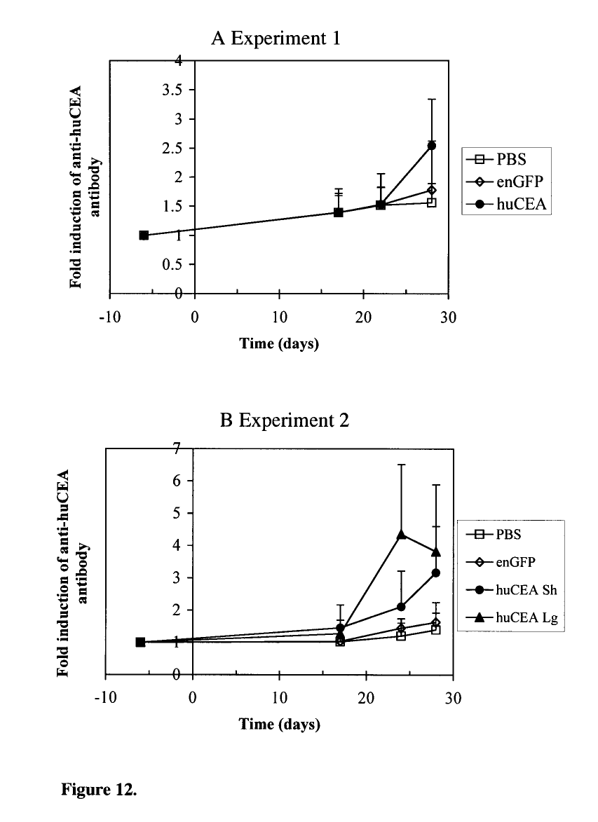

During these two tumor regression experiments (FIGS. 12A and 12B respectively), blood was collected from huCEA Tg mice to check for anti-huCEA antibodies in sera by ELISA and determine whether this vaccination strategy was able to break immune tolerance and induce a humoral response. The antibody levels detected were noticeably higher in the sera of LV-huCEA vaccinated mice only after the boost injection. On average, the OD readings measured at day 28 for sera from mice of the huCEA group were significantly higher than the OD measured before immunization (P<0.05, Student's t-test), whereas day 28 measures were not significantly different from pre-immunization measurements in the PBS and enGFP groups. Due to individual variation, we chose to represent the induction factors at the indicated time points by dividing the OD measured at time (t) by the OD measured from the pre-immunization serum of the same mouse (FIG. 12). Based on the variations in non-immunized mice, we considered induction factors greater or equal to 2.2 as positive. Looking at the individual results, we did not detect any significant anti-huCEA antibody induction in any of the sera from the PBS groups. In each enGFP group, only one mouse showed some increase in anti-huCEA antibody production. On the other hand, induction of anti-huCEA antibody production was observed following the immunizations in the sera from 4/6 and 7/9 LV-huCEA vaccinated mice from the first and second studies respectively. In addition, this relevant anti-huCEA antibody production appeared to correlate with anti-tumor immunity, since there was no antibody induction in the sera from the two LV-huCEA vaccinated mice that did not show any tumor regression. The anti-huCEA antibody induction was higher in the second experiment leading to fold-inductions close to be significantly different between the huCEA and control groups (P=0.06, Student's t-test).

Vaccination with LV-huCEA Induces a Balanced Th1/Th2 Pattern of Cytokine Activation in huCEA-Tg Mice.

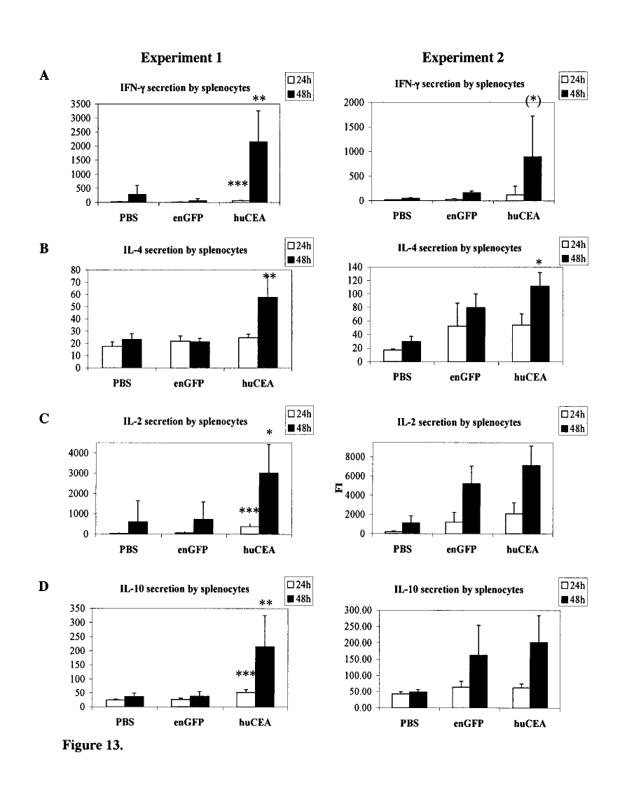

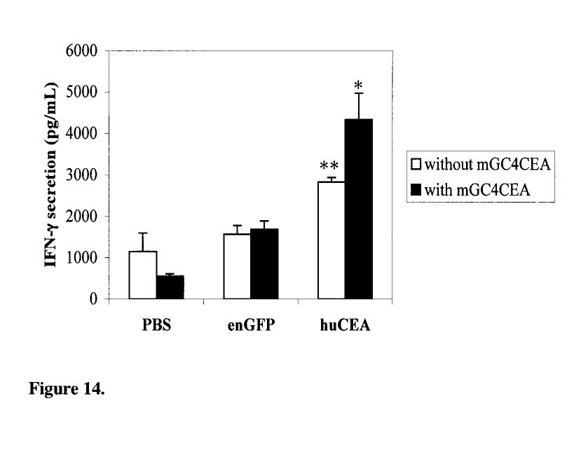

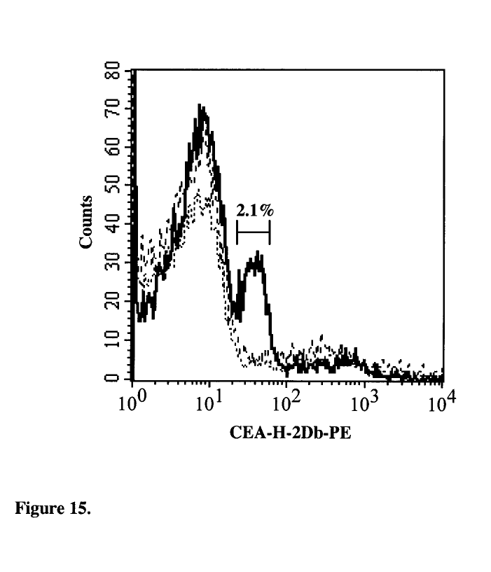

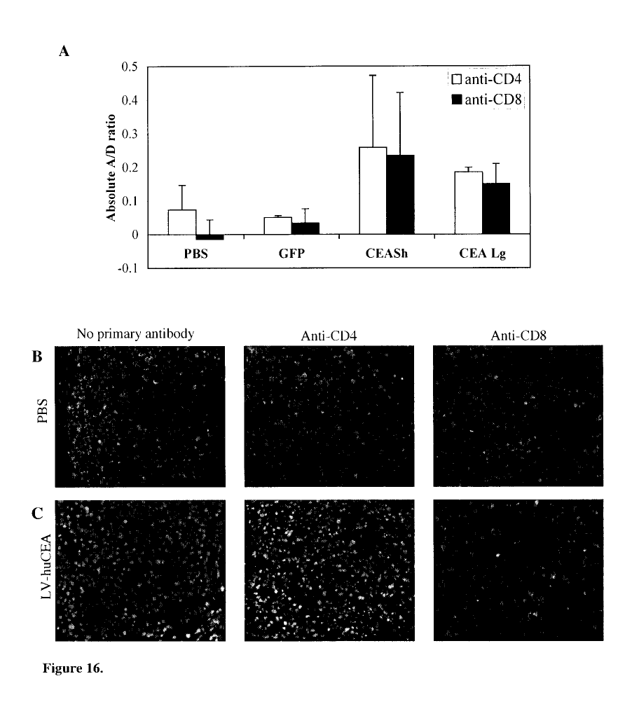

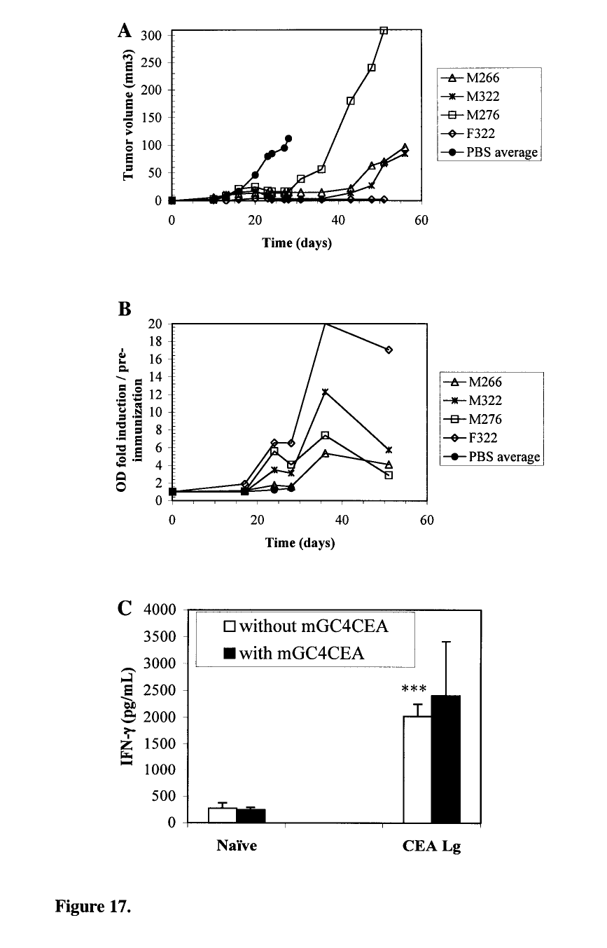

In order to assess the status of the immune system in the vaccinated mice, splenocytes were harvested and cultured individually at a density of 2×10⁶ cells/ml. Twenty-four hour and 48 h supernatants were used for multiple cytokine detection by Luminex. Results revealed that LV-huCEA immunization generated cytokines characteristic of both a humoral and a cellular immune response (FIG. 13). Indeed, a clear increase in the secretion of both IFN-γ, a Th1 type cytokine, and IL-4, a Th2 type cytokine, were detected. The induction of IL-4 secretion following LV-huCEA vaccination was significant compared to the levels measured for splenocytes from control groups in both experiments (FIG. 13B; P<0.05, Student's t-test). Regarding IFN-γ secretion, the difference with control groups was significant in the first experiment only (P<0.05, Student's t-test). However, for the second experiment, the only mouse of the huCEA group that had uninhibited tumor growth did not show any clear induction of IFN-γ secretion, suggesting a link between the Th1 response and the tumor regression. When this non-responsive mouse was removed from analysis, IFN-γ secretion induction for this group reached statistical significance. A significant induction of IL-2 and IL-10 secretion was shown in the first experiment (P<0.05, Student's t-test) (FIGS. 13C and 13D). The increase of secretion by splenocytes of these 4 cytokines (IL-4, IFN-γ, IL-2, and IL-10) was measurable in all mice of the huCEA group of the first experiment, except in the one mouse that did not show tumor growth inhibition. This suggests a link between the immune response induced by LV-huCEA vaccination and the tumor regression observed. In the second experiment, we also detected an increase of IL-2 and IL-10 cytokine secretion that was significant compared to the PBS group levels (p<0.05, Student's t-test). Nevertheless, this induction was not statistically different from the one measured for the enGFP group. Indeed, IL-2 and IL-10 secretion were also slightly stimulated by the LV-enGFP vaccine. Levels of TNF-alpha were more variable and we only detected a trend toward a higher secretion of this cytokine after LV-huCEA vaccination in both experiments (data not shown).

LV-huCEA Vaccinations Induced a huCEA-Specific T Cell Response.

In the second experiment, we wanted to determine whether huCEA-specific T cells had been induced by the LV-huCEA immunizations. Specific T cell activity should be detectable in the presence of target cells expressing huCEA. To show this, we cultured pooled splenocytes from immunized mice alone or in presence of the target mGC4CEA cells for 48 h, and then measured IFN-γ secretion by ELISA. The optimal effector to target cell ratio was found to be 20:1. Splenocytes from LV-huCEA vaccinated mice secreted significantly more IFN-γ than splenocytes from the control group (P<0.005, Student's t-test) (FIG. 14), confirming the general activation status elucidated by quantitation of selected cytokines. In addition, we detected significantly more IFN-γ secreted by the splenocytes from LV-huCEA vaccinated mice when they were in the presence of mGC4CEA target cells (p<0.05, Student's t-test). This did not occur in the other groups. This result suggests the presence of active T cells specific for the huCEA antigen.

In order to detect huCEA-specific cytotoxic T lymphocytes (CTLs) that could be responsible for the anti-tumor activity, we double-stained splenocytes with an APC-conjugated anti-CD8 antibody and a PE-conjugated huCEA/H-2D^{b}-tetramer. It was found that 2.1% CD8⁺ splenocytes were specific for the huCEA peptide (FIG. 15). This data provides evidence for the induction of a huCEA-specific CTL response.

In addition to looking at the immune response status in the spleen, we analyzed the infiltration of immune cells into the tumor. Tumor sections were made from three mice per group and the presence of CD4 and CD8⁺ cells was ascertained by immunofluorescence staining using Alexa488-labeled antibodies. Absolute ratios of Alexa488/DAPI fluorescent fraction areas (absolute AD ratios) were calculated as described in methods and reported per group (FIG. 16A,B). CD4⁺ and CD8⁺ cells were found in all tumors suggesting an infiltration of these cells in most tumors. There is also a clear trend toward an increase in CD4⁺ and CD8⁺ cells following LV-huCEA vaccination; however, significant differences were not shown due to individual variability. That said, no CD8⁺ cell response was detected in the one mouse of the CEASh group that showed a normal tumor growth rate. Removing the data from this mouse lead to a significant difference between the absolute AD ratios of CEASh group compared to the enGFP or PBS groups (P<0.05, Student's t test). The two other mice of the CEASh group which had shown tumor regression were found to have a high absolute AD ratio of CD8⁺ cells (0.22 and 0.43). Such ratios were always less than 0.1 for both anti-CD4 and anti-CD8 analyses of the tumors of the control groups, except for one PBS-injected mouse that showed a 0.15 absolute AD ratio with anti-CD4. These observations suggest a crucial role for CD8⁺ cells in tumor regression. The absolute AD ratio for the CD4⁺ labeling was also higher than 0.2 for 2 of the 3 analyzed tumors of the CEASh group.

the Anti-Tumor Immune Response Induced by Vaccination with LV-huCEA does not Persist Long-Term.

In the second tumor rejection experiment, we also wanted to assess the long-term persistence of the anti-tumor immunity induced by LV-huCEA immunization. To this end, we included an additional group to be followed for two months. This group (huCEA Lg) received a third vaccination, but with a lower dose of 0.1×10⁶ TU. The sizes of all tumors remained stable until 8 days after the last low-dose LV-huCEA injection corresponding to day 36 after tumor cell inoculation. Then tumors resumed growth in all mice between day 36 and day 43, with the exception of one mouse that had a very small stable tumor (less than 3 mm³) for the duration of the experiment (FIG. 17A). Concomitantly with accelerated tumor growth, we measured a rapid decline in the anti-huCEA antibody levels in the sera (FIG. 17B). This correlated with a decrease in IL-4 secretion by splenocytes as measured by the Luminex method (data not shown). In addition, the only mouse that maintained anti-huCEA antibody levels carried a small stable tumor.

The long-term immune response was assessed by assaying splenocytes from two mice injected three times with the LV-huCEA vaccine. At day 57, no huCEA-specific CTL population was detected by tetramer staining of the splenocytes (data not shown). Immunofluorescence staining of tumor sections showed that the absolute AD ratios were intermediate between values obtained for control and CEASh groups (0.11 to 0.19), suggesting a reduction of T cell infiltration (FIG. 16A). When assayed, IFN-γ secretion in the absence of tumor cells by splenocytes from LV-huCEA-vaccinated mice was found to be maintained at the same level as seen for spleen cells from short-term vaccinated mice and was still significantly different from that obtained with splenocytes from naïve mice (p<0.0005, Student's t-test). However, the specific increase of IFN-secretion by the presence of the target cells was no longer detectable (p>0.05, Student's t-test).

Taken together, these data suggested that neither the humoral nor the cellular immune responses induced by LV-huCEA immunization were maintained and that long-term anti-tumor immunity may require booster vaccinations.

### Discussion

In this study, we wanted to assess the potential of injections of low doses of LV as a vaccine directed against the tumor antigen CEA and to characterize subsequent T cell and B cell responses. We demonstrated that direct footpad injections of low doses of LV-huCEA (˜0.15×10⁶ TU) were able to break immune tolerance against huCEA (a true self-antigen in this instance) and induce efficient immunity against CEA-positive tumors in huCEA Tg mice. In addition to regression of the subcutaneous tumors observed in most of the vaccinated mice (66.6%), both cellular and humoral immune responses were induced. Moreover, the tumor regression appeared to correlate with both the antibody and the CTL responses, and was linked to CD8⁺ cell infiltration into the tumors. This finding is in accordance with the observation that CD8⁺ T cells within cancer cell nests were significantly associated with a better survival of patients with colorectal cancer (21).

To our knowledge, this is the first time that tumor regression following delivery of low doses of LV into the draining lymph nodes in mice has been demonstrated. Indeed, previous studies in other non-CEA models used much higher doses of the corresponding LV. Authors do not mention the method used, but as the unit is TU it still should be a functional titer. However, even functional titers can be evaluated by different methods. Here they do flo transduction but on Jurkat cells instead of 293T. For example, one study targeting ovalbumin melanoma antigen showed slowing of tumor growth only, despite using doses of 10⁷ TU per mouse (9). This may be due to the more aggressive tumor model that was used in that case. On the other hand, Kim et al., who were targeting the Neu antigen, were able to get anti-subcutaneous tumor responses similar to our results, but using doses greater than 10⁷ TU per mouse in a non-transgenic mouse model (10). When they used the same strategy in a transgenic model with spontaneous breast tumor development, they showed an increase in survival of 25% (10). Importantly, our study demonstrates for the first time that direct LV-huCEA administration into the footpad led to the establishment of a full immune response, including an antibody response, whereas previous studies only showed CTL responses (9, 10).

Interestingly, both the measurable anti-huCEA antibody response and tumor regression occurred after the second virus injection. This is expected as a secondary immune response may be required to reduce tumor size. In future studies we will evaluate whether multiple LV injections (or co-injection of LVs that engineer expression of other immune modulating factors; as in Mossoba et al, submitted) transform the current transient therapeutic effect into a stable curative modality. As mentioned, the anti-tumor immunity demonstrated persisted long-term in only one mouse. Nevertheless, the finding of long-term tumor stabilization indicates that such vaccination schedules are sufficient in principle to provide long-lasting anti-tumor immunity. This has to be confirmed, however, in larger groups. Establishment of long-lasting anti-tumor immunity might be counteracted by induction of ‘immune-escape’ mechanisms, observed also in different immunotherapy approaches for colorectal cancer (22). There Mazzzolini et al. hypothesised a role for IFN-gamma in this phenomenon: IFN-gamma can stimulate the activity of indoleamine 2,3 dioxygenase, for example, that lowers the concentration of the essential amino acid tryptophan by conversion into immunosuppressive metabolites, leading to suppression of T cell activation. This possibility is supported by our observation of a large increase in IFN-gamma secretion by splenocytes in mice treated with LV-huCEA.

In our study, we observed tumor growth resuming after day 36, one week after the last LV boost. In the study by Kim et al. in which LVs were also used, subcutaneous tumor growth was not followed beyond day 32 (10). Therefore, no information exists whether long-lasting tumor immunity was induced in their experiments. In the Dullaers et al. study, the in vivo CTL activity was shown to be maintained until day 30, however, it was not assessed after this time and mouse survival decreased around day 35 (9). This phenomenon may also be due to the lack of induction of a persistent anti-tumor immune response as observed in our experiments. Nevertheless, different prime-boost approaches can be tried to overcome this limitation.

It is now well established that CD8⁺ T cells are a critical component of the immune response against cancer. Indeed, CTLs play a major role in tumor rejection (23-25). In order to induce a memory response, it is crucial that some of these CD8⁺ T cells survive long-term. Using our strategy, we observed that tumor re-growth was correlated with a disappearance of the specific CTL population that had been detected by huCEA peptide/MHC tetramer staining at day 28. Immunofluorescence staining of tumor sections also suggested a decrease in the CD8⁺ cell infiltration in the tumors of LV-huCEA vaccinated mice in the long-term. We hypothesize that maintenance of anti-CEA CTLs should provide for long-term anti-tumor immunity. Different approaches have been used to achieve this goal and could be tested in combination with our strategy—like the use of low doses of IL-2 to stimulate the proliferation and maintenance of memory CD8⁺ T cells (26,27) or the administration of 4-11B agonists that can rescue CD8⁺ T cells from activation induced cell death (28,29).

In addition to these approaches to directly induce more memory CD8⁺ T cells, many parameters could affect the establishment of long-term anti-tumor immunity. IL-4, a Th2 cytokine typically associated with humoral responses, has proven potential to induce CD8⁺ T cell memory leading to a long-term anti-tumor effect (30). This observation also illustrates the relationship between cellular and humoral immune responses, and the link between the humoral response and the establishment of immune memory. Our data are in line with this theory as the kinetics of the long-term tumor re-growth corresponded to the kinetics of the anti-huCEA antibody response. In addition, the only mouse that showed long-term stable tumor growth restriction also had stable anti-huCEA antibody levels. The role of CD4⁺ helper T cells in the generation of long-term anti-tumor effects has recently been directly demonstrated in vaccinated mice with established tumors (31). Therefore we can hypothesize that targeting the expression of the TAA to the lysosomal compartment, in order to enhance presentation in the context of MHC class II molecules, could improve the long-term outcome of our vaccine strategy. Success has been demonstrated with this approach using the MAGE-A3 antigen (32) and HIV nef mRNA transfer into APCs (33).

Another factor that could explain our difficulty to engineer a persistent immune response is tolerance induced by the tumor environment. It is now well established that regulatory T (Treg) cells constitute a major obstacle to immunotherapy (34). Treg cells are known to be activated by IL-10, which we determined to be strongly secreted by splenocytes following vaccination. Myeloid-derived suppressor cells (MSDCs) represent another tolerance-inducing population. In cancer, these cells accumulate and persist (35), ultimately leading to suppression of T cell responses and development of Treg cells. MDSC accumulation has been linked to inflammation (36). In our study, the high induction of IFN-γ secretion might have induced MDSC accumulation, leading to the long-term disappearance of the immune response. Several ways of dealing with MDSCs are being explored, like promoting MDSCs differentiation by administration of all-trans retinoic acid (35,37) or blockade of tumor-derived stem cell factor, which prevents tumor-specific T cell anergy, Treg cell development, and tumor angiogenesis (38).

Overall, our work showed the potency of footpad vaccinations with low doses of LV-huCEA to induce efficient immunity against huCEA-expressing tumors in huCEA transgenic mice. In addition, since it has been shown that this mode of injection leads to priming of T cells in lymph nodes (14), these data could be extended to clinical applications using intra-nodal injections, an administration route that is showing promise for anti-cancer immunotherapy (39). In addition, our long-term study suggested that our active immunotherapy strategy should be combined with “passive” approaches aiming to improve the long-term effect.

### REFS FOR EXAMPLE 7

1. Mossoba M E, Medin J A. Cancer immunotherapy using virally transduced dendritic cells: Animal studies and human clinical trials. Expert Rev Vaccines 2006 October; 5(5):717-32.
2. Breckpot K, Dullaers M, Bonehill A, et al. Lentivirally transduced dendritic cells as a tool for cancer immunotherapy. J Gene Med 2003 August; 5(8):654-67.
3. Vigna E, Naldini L. Lentiviral vectors: Excellent tools for experimental gene transfer and promising candidates for gene therapy. J Gene Med 2000 September-October; 2(5):308-16.
4. Breckpot K, Aerts J L, Thielemans K. Lentiviral vectors for cancer immunotherapy: Transforming infectious particles into therapeutics. Gene Ther 2007 June; 14(11):847-62.
5. Yang Y, Huang C T, Huang X, Pardoll D M. Persistent toll-like receptor signals are required for reversal of regulatory T cell-mediated CD8 tolerance. Nat Immunol 2004 May; 5(5):508-15.
6. VandenDriessche T, Thorrez L, Naldini L, et al. Lentiviral vectors containing the human immunodeficiency virus type-1 central polypurine tract can efficiently transduce nondividing hepatocytes and antigen-presenting cells in vivo. Blood 2002 Aug. 1; 100(3):813-22.
7. Palmowski M J, Lopes L, Ikeda Y, Salio M, Cerundolo V, Collins M K. Intravenous injection of a lentiviral vector encoding N Y-ESO-1 induces an effective CTL response. J Immunol 2004 Feb. 1; 172(3):1582-7.
8. Esslinger C, Chapatte L, Finke D, et al. In vivo administration of a lentiviral vaccine targets DCs and induces efficient CD8(+) T cell responses. J Clin Invest 2003 June; 111(11):1673-81.
9. Dullaers M, Van Meirvenne S, Heirman C, et al. Induction of effective therapeutic antitumor immunity by direct in vivo administration of lentiviral vectors. Gene Ther 2006 April; 13(7):630-40.
10. Kim J H, Majumder N, Lin H, Watkins S, Falo L D, Jr, You Z. Induction of therapeutic antitumor immunity by in vivo administration of a lentiviral vaccine. Hum Gene Ther 2005 November; 16(11):1255-66.
11. Berinstein N L. Carcinoembryonic antigen as a target for therapeutic anticancer vaccines: A review. J Clin Oncol 2002 Apr. 15; 20(8):2197-207.
12. Lorenz M, Staib-Sebler E, Hochmuth K, et al. Surgical resection of liver metastases of colorectal carcinoma: Short and long-term results. Semin Oncol 2000 October; 27(5 Suppl 10):112-9.
13. Nagorsen D, Thiel E. Clinical and immunologic responses to active specific cancer vaccines in human colorectal cancer. Clin Cancer Res 2006 May 15; 12(10):3064-9.
14. He Y, Zhang J, Donahue C, Falo L D, Jr. Skin-derived dendritic cells induce potent CD8(+) T cell immunity in recombinant lentivector-mediated genetic immunization. Immunity 2006 May; 24(5):643-56.
15. Marshall J L, Gulley J L, Arlen P M, et al. Phase I study of sequential vaccinations with fowlpox-CEA(6D)-TRICOM alone and sequentially with vaccinia-CEA(6D)-TRICOM, with and without granulocyte-macrophage colony-stimulating factor, in patients with carcinoembryonic antigen-expressing carcinomas. J Clin Oncol 2005 Feb. 1; 23(4):720-31.
16. Marshall J L, Hoyer R J, Toomey M A, et al. Phase I study in advanced cancer patients of a diversified prime-and-boost vaccination protocol using recombinant vaccinia virus and recombinant nonreplicating avipox virus to elicit anti-carcinoembryonic antigen immune responses. J Clin Oncol 2000 Dec. 1; 18(23):3964-73.
17. Morse M A, Clay T M, Hobeika A C, et al. Phase I study of immunization with dendritic cells modified with fowlpox encoding carcinoembryonic antigen and costimulatory molecules. Clin Cancer Res 2005 Apr. 15; 11(8):3017-24.
18. Eades-Perner A M, van der Putten H, Hirth A, et al. Mice transgenic for the human carcinoembryonic antigen gene maintain its spatiotemporal expression pattern. Cancer Res 1994 Aug. 1; 54(15):4169-76.
19. Nockel J, van den Engel N K, Winter H, Hatz R A, Zimmermann W, Kammerer R. Characterization of gastric adenocarcinoma cell lines established from CEA424/S V40 T antigen-transgenic mice with or without a human CEA transgene. BMC Cancer 2006 Mar. 14; 6:57.
20. Cusi M G, Del Vecchio M T, Terrosi C, et al. Immune-reconstituted influenza virosome containing CD40L gene enhances the immunological and protective activity of a carcinoembryonic antigen anticancer vaccine. J Immunol 2005 Jun. 1; 174(11):7210-6.
21. Naito Y, Saito K, Shiiba K, et al. CD8+ T cells infiltrated within cancer cell nests as a prognostic factor in human colorectal cancer. Cancer Res 1998 Aug. 15; 58(16):3491-4.
22. Mazzolini G, Murillo O, Atorrasagasti C, et al. Immunotherapy and immunoescape in colorectal cancer. World J Gastroenterol 2007 Nov. 28; 13(44):5822-31.
23. Hung C F, Cheng W F, He L, et al. Enhancing major histocompatibility complex class I antigen presentation by targeting antigen to centrosomes. Cancer Res 2003 May 15; 63(10):2393-8.
24. Pilon S A, Piechocki M P, Wei W Z. Vaccination with cytoplasmic ErbB-2 DNA protects mice from mammary tumor growth without anti-ErbB-2 antibody. J Immunol 2001 Sep. 15; 167(6):3201-6.
25. Xu D, Gu P, Pan P Y, Li Q, Sato A I, Chen S H. N K and CD8+ T cell-mediated eradication of poorly immunogenic B16-F10 melanoma by the combined action of I L-12 gene therapy and 4-1 B B costimulation. Int J Cancer 2004 Apr. 20; 109(4):499-506.
26. Manjunath N, Shankar P, Wan J, et al. Effector differentiation is not prerequisite for generation of memory cytotoxic T lymphocytes. J Clin Invest 2001 September; 108(6):871-8.
27. He H, Wisner P, Yang G, et al. Combined I L-21 and low-dose I L-2 therapy induces anti-tumor immunity and long-term curative effects in a murine melanoma tumor model. J Transl Med 2006 Jun. 13; 4:24.
28. Takahashi C, Mittler R S, Vella A T. Cutting edge: 4-1 B B is a bona fide CD8 T cell survival signal. J Immunol 1999 May 1; 162(9):5037-40.
29. Li B, Lin J, Vanroey M, Jure-Kunkel M, Jooss K. Established B16 tumors are rejected following treatment with G M-CSF-secreting tumor cell immunotherapy in combination with anti-4-1B B mAb. Clin Immunol 2007 October; 125(1):76-87.
30. Huang L R, Chen F L, Chen Y T, Lin Y M, Kung J T. Potent induction of long-term CD8+ T cell memory by short-term I L-4 exposure during T cell receptor stimulation. Proc Natl Acad Sci USA 2000 Mar. 28; 97(7):3406-11.
31. Lin C T, Chang T C, Shaw S W, et al. Maintenance of CD8 effector T cells by CD4 helper T cells eradicates growing tumors and promotes long-term tumor immunity. Vaccine 2006 Sep. 11; 24(37-39):6199-207.
32. Bonehill A, Heirman C, Tuyaerts S, et al. Messenger RNA-electroporated dendritic cells presenting MAGE-A3 simultaneously in HLA class I and class I I molecules. J Immunol 2004 Jun. 1; 172(11):6649-57.
33. Kavanagh D G, Kaufmann D E, Sunderji S, et al. Expansion of HIV-specific CD4+ and CD8+ T cells by dendritic cells transfected with mRNA encoding cytoplasm- or lysosome-targeted nef. Blood 2006 Mar. 1; 107(5):1963-9.
34. Wilczynski J R, Kalinka J, Radwan M. The role of T-regulatory cells in pregnancy and cancer. Front Biosci 2008 Jan. 1; 13:2275-89.
35. Marx J. Cancer immunology. cancers bulwark against immune attack: MDS cells. Science 2008 Jan. 11; 319(5860):154-6.
36. Bunt S K, Yang L, Sinha P, Clements V K, Leips J, Ostrand-Rosenberg S. Reduced inflammation in the tumor microenvironment delays the accumulation of myeloid-derived suppressor cells and limits tumor progression. Cancer Res 2007 Oct. 15; 67(20):10019-26.
37. Nefedova Y, Fishman M, Sherman S, Wang X, Beg A A, Gabrilovich D I. Mechanism of all-trans retinoic acid effect on tumor-associated myeloid-derived suppressor cells. Cancer Res 2007 Nov. 15; 67(22):11021-8.
38. Pan P Y, Wang G X, Yin B, et al. Reversion of immune tolerance in advanced malignancy: Modulation of myeloid-derived suppressor cell development by blockade of stem-cell factor function. Blood 2008 Jan. 1; 111(1):219-28.
39. Spaner D E, Astsaturov I, Vogel T, et al. Enhanced viral and tumor immunity with intranodal injection of canary pox viruses expressing the melanoma antigen, gp100. Cancer 2006 Feb. 15; 106(4):890-9.

### REFERENCES

### Round Bracket Refs

1) Chakrabarti et al. (2004) Vaccine 22 (9-10), pp. 1199-1205.
2) Johansson et al. (2006) Vaccine 24 (21), pp. 4572-4575
3) Samanci et al. (1998) Cancer Immunology Immunotherapy 47 (3), pp. 131-142
4) Ullenhag et al. (2004) Clinical Cancer Research 10 (10), pp. 3273-3281
5) Kantor et al. (1992) Journal of the National Cancer Institute 84 (14), pp. 1084-1091 O R Cancer Research 52 (24), pp. 6917-6925
6) Hodge et al. (1997) Vaccine 15 (6-7), pp. 759-768
7) Greiner et al. (2002) Cancer Research 62 (23), pp. 6944-6951
8) Nair et al. (1999) International Journal of Cancer 82 (1), pp. 121-124
9) Liu et al. (2004) Clinical Cancer Research 10 (8), pp. 2645-2651
10) Matsuda et al. (2004) Cancer Immunology, Immunotherapy 53 (7), pp. 609-616
11) Cho et al. (2003) Vaccine 22 (2), pp. 224-236
12) Oh et al. (2006) Vaccine 24 (15), pp. 2860-2868
13) Mc Aneny et al. (1996) Annals of Surgical Oncology 3 (5), pp. 495-500
14) Marshall et al. (1999) Journal of Clinical Oncology 17 (1), pp. 332-337
15) von Mehren et al. (2000) Clinical Cancer Research 6 (6), pp. 2219-2228
16) Marshall et al. (2005) Hance et al. (2005) Journal of Clinical Oncology 23 (4), pp. 720-731
17) Thompson et al. (2000) International Journal of Cancer 86 (6), pp. 863-869.

### UNBRACKETED REFERENCES

### Superscript

1. Guilhot F, Roy L, Guilhot J, Millot F. Interferon therapy in chronic myelogenous leukemia. Hematol Oncol Clin North Am. 2004; 18:585-603, viii.
2. Pyrhonen S O. Systemic therapy in metastatic renal cell carcinoma. Scand J Surg. 2004; 93:156-161.
3. Nemunaitis J. GVAX (GMCSF gene modified tumor vaccine) in advanced stage non small cell lung cancer. J Control Release. 2003; 91:225-231.
4. Portielje J E, Gratama J W, van Ojik H H, Stoter G, Kruit W H. IL-12: a promising adjuvant for cancer vaccination. Cancer Immunol Immunother. 2003; 52:133-144.
5. Sasiain M C, de la Barrera S, Fink S, et al. Interferon-gamma (IFN-gamma) and tumour necrosis factor-alpha (TNF-alpha) are necessary in the early stages of induction of CD4 and CD8 cytotoxic T cells by Mycobacterium leprae heat shock protein (hsp) 65 kD. Clin Exp Immunol. 1998; 114:196-203.
6. Portielje J E, Lamers C H, Kruit W H, et al. Repeated administrations of interleukin (IL)-12 are associated with persistently elevated plasma levels of IL-10 and declining IFN-gamma, tumor necrosis factor-alpha, IL-6, and IL-8 responses. Clin Cancer Res. 2003; 9:76-83.
7. Sacco S, Heremans H, Echtenacher B, et al. Protective effect of a single interleukin-12 (IL-12) predose against the toxicity of subsequent chronic IL-12 in mice: role of cytokines and glucocorticoids. Blood. 1997; 90:4473-4479.
8. Leonard J P, Sherman M L, Fisher G L, et al. Effects of single-dose interleukin-12 exposure on interleukin-12-associated toxicity and interferon-gamma production. Blood. 1997; 90:2541-2548.

### SQUARE BRACKET REFS

1. Vari, F, Hart D N. (2004). Loading DCs with ag. Cytotherapy; 6: 111-21.
2. Banchereau, J, Ueno H, Dhodapkar M, Connolly J, Finholt J P, Klechevsky E, et al. (2005). Immune and clinical outcomes in patients with stage I V melanoma vaccinated with peptide-pulsed dendritic cells derived from CD34+ progenitors and activated with type I interferon. J Immunother; 28: 505-16.
3. Lee, W C, Wang H C, Hung C F, Huang P F, Lia C R, Chen M F. (2005). Vaccination of advanced hepatocellular carcinoma patients with tumor lysate-pulsed dendritic cells: A clinical trial. J Immunother; 28: 496-504.
4. Lesterhuis, W J, de Vries I J, Schuurhuis D H, Boullart A C, Jacobs J F, de Boer A J, et al. (2006). Vaccination of colorectal cancer patients with CEA-loaded dendritic cells: Antigen-specific T cell responses in DTH skin tests. Ann Oncol; 17: 974-80.
5. Liu, K J, Wang C C, Chen L T, Cheng A L, Lin D T, Wu Y C, et al. (2004). Generation of carcinoembryonic antigen (CEA)-specific T-cell responses in HLA-A*0201 and HLA-A*2402 late-stage colorectal cancer patients after vaccination with dendritic cells loaded with CEA peptides. Clin Cancer Res; 10: 2645-51.
6. Boczkowski, D, Nair S K, Nam J H, Lyerly H K, Gilboa E. (2000). Induction of tumor immunity and cytotoxic T lymphocyte responses using dendritic cells transfected with messenger RNA amplified from tumor cells. Cancer Res; 60: 1028-34.
7. Boczkowski, D, Nair S K, Snyder D, Gilboa E. (1996). Dendritic cells pulsed with RNA are potent antigen-presenting cells in vitro and in vivo. J Exp Med; 184: 465-72.
8. Nair, S K, Boczkowski D, Morse M, Cumming R I, Lyerly H K, Gilboa E. (1998). Induction of primary carcinoembryonic antigen (CEA)-specific cytotoxic T lymphocytes in vitro using human dendritic cells transfected with RNA. Nat Biotechnol; 16: 364-9.
9. Zhou, Y, Bosch M L, Salgaller M L. (2002). Current methods for loading dendritic cells with tumor antigen for the induction of antitumor immunity. J Immunother; 25: 289-303.
10. He, Y, Zhang J, Mi Z, Robbins P, Falo L D, Jr. (2005). Immunization with lentiviral vector-transduced dendritic cells induces strong and long-lasting T cell responses and therapeutic immunity. J Immunol; 174: 3808-17.
11. He, Y, Zhang J, Donahue C, Falo L D, Jr. (2006). Skin-derived dendritic cells induce potent CD8(+) T cell immunity in recombinant lentivector-mediated genetic immunization. Immunity; 24: 643-56.
12. Firat, H, Zennou V, Garcia-Pons F, Ginhoux F, Cochet M, Danos O, et al. (2002). Use of a lentiviral flap vector for induction of CTL immunity against melanoma. perspectives for immunotherapy. J Gene Med; 4: 38-45.
13. Breckpot, K, Dullaers M, Bonehill A, van Meirvenne S, Heirman C, de Greef C, et al. (2003). Lentivirally transduced dendritic cells as a tool for cancer immunotherapy. J Gene Med; 5: 654-67.
14. Metharom, P, Ellem K A, Schmidt C, Wei M Q. (2001). Lentiviral vector-mediated tyrosinase-related protein 2 gene transfer to dendritic cells for the therapy of melanoma. Hum Gene Ther; 12: 2203-13.
15. Yoshimitsu, M, Sato T, Tao K, Walia J S, Rasaiah V I, Sleep G T, et al. (2004). Bioluminescent imaging of a marking transgene and correction of fabry mice by neonatal injection of recombinant lentiviral vectors. Proceedings of the National Academy of Sciences; 101: 16909-14.
16. Mossoba, M E, Medin J A. (2006). Cancer immunotherapy using virally-transduced DCs: A review of animal studies and human clinical trials. Expert Review of Vaccines; 5: 717-732.
17. Osman, I, Scher H I, Drobnjak M, Verbel D, Morris M, Agus D, et al. (2001). HER-2/neu (p185neu) protein expression in the natural or treated history of prostate cancer. Clin Cancer Res; 7: 2643-7.
18. Cobleigh, M A, Vogel C L, Tripathy D, Robert N J, Scholl S, Fehrenbacher L, et al. (1999). Multinational study of the efficacy and safety of humanized anti-HER2 monoclonal antibody in women who have HER2-overexpressing metastatic breast cancer that has progressed after chemotherapy for metastatic disease. J Clin Oncol; 17: 2639-48.
19. Hirsch, F R, Franklin W A, Veve R, Varella-Garcia M, Bunn P A, Jr. (2002). HER2/neu expression in malignant lung tumors. Semin Oncol; 29: 51-8.
20. Hellstrom, I, Goodman G, Pullman J, Yang Y, Hellstrom K E. (2001). Overexpression of HER-2 in ovarian carcinomas. Cancer Res; 61: 2420-3.
21. Scott, G K, Robles R, Park J W, Montgomery P A, Daniel J, Holmes W E, et al. (1993). A truncated intracellular HER2/neu receptor produced by alternative RNA processing affects growth of human carcinoma cells. Mol Cell Biol; 13: 2247-57.
22. Chhikara, M, Huang H, Vlachaki M T, Zhu X, Teh B, Chiu K J, et al. (2001). Enhanced therapeutic effect of HSV-tk+GCV gene therapy and ionizing radiation for prostate cancer. Mol Ther; 3: 536-42.
23. Medin, J A, Liang S B, Hou J W, Kelley L S, Peace D J, Fowler D H. (2005). Efficient transfer of PSA and PSMA cDNAs into DCs generates antibody and T cell antitumor responses in vivo. Cancer Gene Ther; 12: 540-51.
24. Dullaers, M, Breckpot K, Van Meirvenne S, Bonehill A, Tuyaerts S, Michiels A, et al. (2004). Side-by-side comparison of lentivirally transduced and mRNA-electroporated dendritic cells: Implications for cancer immunotherapy protocols. Mol Ther; 10: 768-79.
25. Chan, T, Sami A, E I-Gayed A, Guo X, Xiang J. (2006). HER-2/neu-gene engineered dendritic cell vaccine stimulates stronger HER-2/neu-specific immune responses compared to DNA vaccination. Gene Ther; 13: 1391-402.
26. Chen, Z, Huang H, Chang T, Carlsen S, Saxena A, Marr R, et al. (2002). Enhanced HER-2/neu-specific antitumor immunity by cotransduction of mouse dendritic cells with two genes encoding HER-2/neu and alpha tumor necrosis factor. Cancer Gene Ther; 9: 778-86.
27. Sakai, Y, Morrison B J, Burke J D, Park J M, Terabe M, Janik J E, et al. (2004). Vaccination by genetically modified dendritic cells expressing a truncated neu oncogene prevents development of breast cancer in transgenic mice. Cancer Res; 64: 8022-8.
28. zum Buschenfelde, C M, Metzger J, Hermann C, Nicklisch N, Peschel C, Bernhard H. (2001). The generation of both T killer and th cell clones specific for the tumor-associated antigen HER2 using retrovirally transduced dendritic cells. J Immunol; 167: 1712-9.
29. Sato, T, Neschadim A, Konrad M, Fowler D H, Lavie A, Medin J A. (2007). Engineered human tmpk/AZT as a novel Enzyme/Prodrug axis for suicide gene therapy. Mol Ther; 15: 962-70.
30. Freeman, T, Dixon A, Campbell E, Tait T, Richardson P, Rice K, et al. (1998). Expression mapping of mouse genes. MGI Direct Data Submission;.
31. Morgan, D J, Kreuwel H T, Fleck S, Levitsky H I, Pardoll D M, Sherman L A. (1998). Activation of low avidity CTL specific for a self epitope results in tumor rejection but not autoimmunity. J Immunol; 160: 643-51.
32. Schreurs, M W, Eggert A A, de Boer A J, Vissers J L, van Hall T, Offringa R, et al. (2000). Dendritic cells break tolerance and induce protective immunity against a melanocyte differentiation antigen in an autologous melanoma model. Cancer Res; 60: 6995-7001.
33. Bronte, V, Apolloni E, Ronca R, Zamboni P, Overwijk W W, Surman D R, et al. (2000). Genetic vaccination with “self” tyrosinase-related protein 2 causes melanoma eradication but not vitiligo. Cancer Res; 60: 253-8.
34. Siatskas, C, Underwood J, Ramezani A, Hawley R G, Medin J A. (2005). Specific pharmacological dimerization of KDR in lentivirally transduced human hematopoietic cells activates anti-apoptotic and proliferative mechanisms. FASEB J; 19: 1752-4.
35. Naldini, L, Blomer U, Gage F H, Trono D, Verma I M. (1996). Efficient transfer, integration, and sustained long-term expression of the transgene in adult rat brains injected with a lentiviral vector. Proc Natl Acad Sci USA; 93: 11382-8.
36. Takenaka, T, Qin G, Brady R O, Medin J A. (1999). Circulating alpha-galactosidase A derived from transduced bone marrow cells: Relevance for corrective gene transfer for fabry disease. Hum Gene Ther; 10: 1931-9.
37. Lutz, M B, Kukutsch N, Ogilvie A L, Rossner S, Koch F, Romani N, et al.
(1999). An advanced culture method for generating large quantities of highly pure dendritic cells from mouse bone marrow. J Immunol Methods; 223: 77-92.
38. Piechocki, M P, Pilon S A, Wei W Z. (2002). Quantitative measurement of anti-ErbB-2 antibody by flow cytometry and ELISA. J Immunol Methods; 259: 33-42.
39. Maecker, H T, Moon J, Bhatia S, Ghanekar S A, Maino V C, Payne J K, et al.
(2005). Impact of cryopreservation on tetramer, cytokine flow cytometry, and ELISPOT. BMC Immunol; 6: 17.

Sequences

SEQ ID NO: 1 LYSOSOMAL TARGETING SEQUENCE
Met Gln Leu Arg Asn Pro Glu Leu His Leu Gly Cys Ala Leu Ala
Leu Arg Phe Leu Ala Leu Val Ser Trp Asp Ile Pro Gly Ala Arg
Ala
SEQ ID NO: 2 CPPT

| | |
|---|---|
| ttttaaaaga aaagggggga ttggggggta cagtgcaggg gaaagaatag tagacataat | 60 |
| agcaacagac atacaaacta aagaattaca aaaacaaatt acaaaaattc aaaatttt | 118 |

SEQ ID NO: 3
Woodchuck Hepatitus Virus WPRE

| | |
|---|---|
| aatcaacctc tggattacaa aatttgtgaa agattgactg gtattcttaa ctatgttgct | 60 |
| ccttttacgc tatgtggata cgctgcttta atgcctttgt atcatgctat tgcttcccgt | 120 |
| atggctttca ttttctcctc cttgtataaa tcctggttgc tgtctcttta tgaggagttg | 180 |
| tggcccgttg tcaggcaacg tggcgtggtg tgcactgtgt ttgctgacgc aacccccact | 240 |
| ggttggggca ttgccaccac ctgtcagctc ctttccggga ctttcgcttt ccccctccct | 300 |
| attgccacgg cggaactcat cgccgcctgc cttgcccgct gctggacagg ggctcggctg | 360 |
| ttgggcactg acaattccgt ggtgttgtcg gggaagctga cgtcctttcc atggctgctc | 420 |
| gcctgtgttg ccacctggat tctgcgcggg acgtccttct gctacgtccc ttcggccctc | 480 |
| aatccagcgg accttccttc ccgcggcctg ctgccggctc tgcggcctct tccgcgtctt | 540 |
| cgccttcgcc ctcagacgag tcggatctcc ctttgggccg cctccccgcc tg | 592 |

SEQ ID NO: 1
pHR′.cPPT.EF.CD19ATmpkF105YR200A.WPRE.SIN.
AATTACCTGTGGTTTCATTTACTCTAAACCTGTGATTCCTCTGAATTATTTTCATTTTAAAGAAATTGT
ATTTGTTAAATATGTACTACAAACTTAGTAGTTGGAAGGGCTAATTCACTCCCAAAGAAGACAAGATAT
CCTTGATCTGTGGATCTACCACACACAAGGCTACTTCCCTGATTAGCAGAACTACACACCAGGGCCAGG
GGTCAGATATCCACTGACCTTTGGATGGTGCTACAAGCTAGTACCAGTTGAGCCAGATAAGGTAGAAGA
GGCCAATAAAGGAGAGAACACCAGCTTGTTACACCCTGTGAGCCTGCATGGGATGGATGACCCGGAGAG
AGAAGTGTTAGAGTGGAGGTTTGACAGCCGCCTAGCATTTCATCACGTGGCCCGAGAGCTGCATCCGGA
GTACTTCAAGAACTGCTGATATCGAGCTTGCTACAAGGGACTTTCCGCTGGGGACTTTCCAGGGAGGCG
TGGCCTGGGCGGGACTGGGGAGTGGCGAGCCCTCAGATCCTGCATATAAGCAGCTGCTTTTTGCCTGTA
CTGGGTCTCTCTGGTTAGACCAGATCTGAGCCTGGGAGCTCTCTGGCTAACTAGGGAACCCACTGCTTA
AGCCTCAATAAAGCTTGCCTTGAGTGCTTCAAGTAGTGTGTGCCCGTCTGTTGTGTGACTCTGGTAACT
AGAGATCCCTCAGACCCTTTTAGTCAGTGTGGAAAATCTCTAGCAGTGGCGCCCGAACAGGGACTTGAA
AGCGAAAGGGAAACCAGAGGAGCTCTCTCGACGCAGGACTCGGCTTGCTGAAGCGCGCACGGCAAGAGG
CGAGGGGCGGCGACTGGTGAGTACGCCAAAAATTTTGACTAGCGGAGGCTAGAAGGAGAGAGATGGGTG
CGAGAGCGTCAGTATTAAGCGGGGGAGAATTAGATCGCGATGGGAAAAAATTCGGTTAAGGCCAGGGGG
AAAGAAAAAATATAAATTAAAACATATAGTATGGGCAAGCAGGGAGCTAGAACGATTCGCAGTTAATCC
TGGCCTGTTAGAAACATCAGAAGGCTGTAGACAAATACTGGGACAGCTACAACCATCCCTTCAGACAGG
ATCAGAAGAACTTAGATCATTATATAATACAGTAGCAACCCTCTATTGTGTGCATCAAAGGATAGAGAT
AAAAGACACCAAGGAAGCTTTAGACAAGATAGAGGAAGAGCAAAACAAAAGTAAGACCACCGCACAGCA
AGCGGCCGCTGATCTTCAGACCTGGAGGAGGAGATATGAGGGACAATTGGAGAAGTGAATTATATAAAT
ATAAAGTAGTAAAAATTGAACCATTAGGAGTAGCACCCACCAAGGCAAAGAGAAGAGTGGTGCAGAGAG
AAAAAAGAGCAGTGGGAATAGGAGCTTTGTTCCTTGGGTTCTTGGGAGCAGCAGGAAGCACTATGGGCG
CAGCGTCAATGACGCTGACGGTACAGGCCAGACAATTATTGTCTGGTATAGTGCAGCAGCAGAACAATT
TGCTGAGGGCTATTGAGGCGCAACAGCATCTGTTGCAACTCACAGTCTGGGGCATCAAGCAGCTCCAGG
CAAGAATCCTGGCTGTGGAAAGATACCTAAAGGATCAACAGCTCCTGGGGATTTGGGGTTGCTCTGGAA
AACTCATTTGCACCACTGCTGTGCCTTGGAATGCTAGTTGGAGTAATAAATCTCTGGAACAGATTTGGA
ATCACACGACCTGGATGGAGTGGGACAGAGAAATTAACAATTACACAAGCTTAATACACTCCTTAATTG
AAGAATCGCAAAACCAGCAAGAAAAGAATGAACAAGAATTATTGGAATTAGATAAATGGGCAAGTTTGT
GGAATTGGTTTAACATAACAAATTGGCTGTGGTATATAAAATTATTCATAATGATAGTAGGAGGCTTGG
TAGGTTTAAGAATAGTTTTTGCTGTACTTTCTATAGTGAATAGAGTTAGGCAGGGATATTCACCATTAT
CGTTTCAGACCCACCTCCCAACCCCGAGGGGACCCGACAGGCCCGAAGGAATAGAAGAAGAAGGTGGAG
AGAGAGACAGAGACAGATCCATTCGATTAGTGAACGGATCTCGACGGTATCGATTTTAAAAGAAAAGGG
GGGATTGGGGGGTACAGTGCAGGGGAAAGAATAGTAGACATAATAGCAACAGACATACAAACTAAAGAA
TTACAAAAACAAATTACAAAAATTCAAAATTTTATCGATAAGCTTTGCAAAGATGGATAAAGTTTTAAA
CAGAGAGGAATCTTTGCAGCTAATGGACCTTCTAGGTCTTGAAAGGAGTGGGAATTGGCTCCGGTGCCC
GTCAGTGGGCAGAGCGCACATCGCCCACAGTCCCCGAGAAGTTGGGGGGAGGGGTCGGCAATTGAACCG
GTGCCTAGAGAAGGTGGCGCGGGGTAAACTGGGAAAGTGATGTCGTGTACTGGCTCCGCCTTTTTCCCG
AGGGTGGGGGAGAACCGTATATAAGTGCAGTAGTCGCCGTGAACGTTCTTTTTCGCAACGGGTTTGCCG
CCAGAACACAGGTAAGTGCCGTGTGTGGTTCCCGCGGGCCTGGCCTCTTTACGGGTTATGGCCCTTGCG
TGCCTTGAATTACTTCCACCTGGCTGCAGTACGTGATTCTTGATCCCGAGCTTCGGGTTGGAAGTGGGT
GGGAGAGTTCGAGGCCTTGCGCTTAAGGAGCCCCTTCGCCTCGTGCTTGAGTTGAGGCCTGGCCTGGGC
GCTGGGGCCGCCGCGTGCGAATCTGGTGGCACCTTCGCGCCTGTCTCGCTGCTTTCGATAAGTCTCTAG
CCATTTAAAATTTTTGATGACCTGCTGCGACGCTTTTTTTCTGGCAAGATAGTCTTGTAAATGCGGGCC
AAGATCTGCACACTGGTATTTCGGTTTTTGGGGCCGCGGGCGGCGACGGGGCCCGTGCGTCCCAGCGCA
CATGTTCGGCGAGGCGGGGCCTGCGAGCGCGGCCACCGAGAATCGGACGGGGGTAGTCTCAAGCTGGCC
GGCCTGCTCTGGTGCCTGGCCTCGCGCCGCCGTGTATCGCCCCGCCCTGGGCGGCAAGGCTGGCCCGGT
CGGCACCAGTTGCGTGAGCGGAAAGATGGCCGCTTCCCGGCCCTGCTGCAGGGAGCTCAAAATGGAGGA
CGCGGCGCTCGGGAGAGCGGGCGGGTGAGTCACCCACACAAAGGAAAAGGGCCTTTCCGTCCTCAGCCG
TCGCTTCATGTGACTCCACGGAGTACCGGGCGCCGTCCAGGCACCTCGATTAGTTCTCGAGCTTTTGGA
GTACGTCGTCTTTAGGTTGGGGGGAGGGGTTTTATGCGATGGAGTTTCCCCACACTGAGTGGGTGGAGA
CTGAAGTTAGGCCAGCTTGGCACTTGATGTAATTCTCCTTGGAATTTGCCCTTTTTGAGTTTGGATCTT
GGTTCATTCTCAAGCCTCAGACAGTGGTTCAAAGTTTTTTTCTTCCATTTCAGGTGTCGTGAGGAATTC
ATGCCACCTCCTCGCCTCCTCTTCTTCCTCCTCTTCCTCACCCCCATGGAAGTCAGGCCCGAGGAACCT
CTAGTGGTGAAGGTGGAAGAGGGAGATAACGCTGTGCTGCAGTGCCTCAAGGGGACCTCAGATGGCCCC
ACTCAGCAGCTGACCTGGTCTCGGGAGTCCCCGCTTAAACCCTTCTTAAAACTCAGCCTGGGGCTGCCA
GGCCTGGGAATCCACATGAGGCCCCTGGCATCCTGGCTTTTCATCTTCAACGTCTCTCAACAGATGGGG
GGCTTCTACCTGTGCCAGCCGGGGCCCCCCTCTGAGAAGGCCTGGCAGCCTGGCTGGACAGTCAATGTG
GAGGGCAGCGGGGAGCTGTTCCGGTGGAATGTTTCGGACCTAGGTGGCCTGGGCTGTGGCCTGAAGAAC
AGGTCCTCAGAGGGCCCCAGCTCCCCTTCCGGGAAGCTCATGAGCCCCAAGCTGTATGTGTGGGCCAAA
GACCGCCCTGAGATCTGGGAGGGAGAGCCTCCGTGTGTCCCACCGAGGGACAGCCTGAACCAGAGCCTC
AGCCAGGACCTCACCATGGCCCCTGGCTCCACACTCTGGCTGTCCTGTGGGGTACCCCCTGACTCTGTG
TCCAGGGGCCCCCTCTCCTGGACCCATGTGCACCCCAAGGGGCCTAAGTCATTGCTGAGCCTAGAGCTG
AAGGACGATCGCCCGGCCAGAGATATGTGGGTAATGGAGACGGGTCTGTTGTTGCCCCGGGCCACAGCT
CAAGACGCTGGAAAGTATTATTGTCACCGTGGCAACCTGACCATGTCATTCCACCTGGAGATCACTGCT
CGGCCAGTACTATGGCACTGGCTGCTGAGGACTGGTGGCTGGAAGGTCTCAGCTGTGACTTTGGCTTAT
CTGATCTTCTGCCTGTGTTCCCTTGTGGGCATTCTTCATCTTGCCGGCGGGGCTGCAGGGATGGCGGCC
CGGCGCGGGGCTCTCATAGTGCTGGAGGGCGTGGACCGCGCCGGGAAGAGCACGCAGAGCCGCAAGCTG
GTGGAAGCGCTGTGCGCCGCGGGCCACCGCGCCGAACTGCTCCGGTTCCCGGAAAGATCAACTGAAATC
GGCAAACTTCTGAGTTCCTACTTGCAAAAGAAAAGTGACGTGGAGGATCACTCGGTGCACCTGCTTTTT
TCTGCAAATCGCTGGGAACAAGTGCCGTTAATTAAGGAAAAGTTGAGCCAGGGCGtGACCCTCGTCGTG
GACAGATACGCATTTTCTGGTGTGGCCTACACaGGTGCCAAGGAGAATTTTTCCCTAGACTGGTGTAAA
CAGCCAGACGTGGGCCTTCCCAAACCCGACCTGGTCCTGTTCCTCCAGTTACAGCTGGCGGATGCTGCC
AAGCGGGGAGCGTTTGGCCATGAGCGCTATGAGAACGGGGCTTTCCAGGAGCGGGCGCTCCGGTGTTTC
CACCAGCTCATGAAAGACACGACTTTGAACTGGAAGATGGTGGATGCTTCCAAAAGCATCGAAGCTGTC
CATGAGGACATCCGCGTGCTCTCTGAGGACGCCATCGCCACTGCCACAGAGAAGCCGCTGGGGGAGCTA
TGGAAGTGAGGATCCAAGCTTCAATTGTGGTCACTCGACAATCAACCTCTGGATTACAAAATTTGTGAA
AGATTGACTGGTATTCTTAACTATGTTGCTCCTTTTACGCTATGTGGATACGCTGCTTTAATGCCTTTG
TATCATGCTATTGCTTCCCGTATGGCTTTCATTTTCTCCTCCTTGTATAAATCCTGGTTGCTGTCTCTT
TATGAGGAGTTGTGGCCCGTTGTCAGGCAACGTGGCGTGGTGTGCACTGTGTTTGCTGACGCAACCCCC
ACTGGTTGGGGCATTGCCACCACCTGTCAGCTCCTTTCCGGGACTTTCGCTTTCCCCCTCCCTATTGCC
ACGGCGGAACTCATCGCCGCCTGCCTTGCCCGCTGCTGGACAGGGGCTCGGCTGTTGGGCACTGACAAT
TCCGTGGTGTTGTCGGGGAAGCTGACGTCCTTTCCATGGCTGCTCGCCTGTGTTGCCACCTGGATTCTG
CGCGGGACGTCCTTCTGCTACGTCCCTTCGGCCCTCAATCCAGCGGACCTTCCTTCCCGCGGCCTGCTG
CCGGCTCTGCGGCCTCTTCCGCGTCTTCGCCTTCGCCCTCAGACGAGTCGGATCTCCCTTTGGGCCGCC
TCCCCGCCTGCTCGAGACCTAGAAAAACATGGAGCAATCACAAGTAGCAATACAGCAGCTACCAATGCT
GATTGTGCCTGGCTAGAAGCACAAGAGGAGGAGGAGGTGGGTTTTCCAGTCACACCTCAGGTACCTTTA
AGACCAATGACTTACAAGGCAGCTGTAGATCTTAGCCACTTTTTAAAAGAAAAGGGGGGACTGGAAGGG
CTAATTCACTCCCAACGAAGACAAGATCTGCTTTTTGCTTGTACTGGGTCTCTCTGGTTAGACCAGATC
TGAGCCTGGGAGCTCTCTGGCTAACTAGGGAACCCACTGCTTAAGCCTCAATAAAGCTTGCCTTGAGTG
CTTCAAGTAGTGTGTGCCCGTCTGTTGTGTGACTCTGGTAACTAGAGATCCCTCAGACCCTTTTAGTCA
GTGTGGAAAATCTCTAGCAGTAGTAGTTCATGTCATCTTATTATTCAGTATTTATAACTTGCAAAGAAA
TGAATATCAGAGAGTGAGAGGCCTTGACATTATAATAGATTTAGCAGGAATTGAACTAGGAGTGGAGCA
CACAGGCAAAGCTGCAGAAGTACTTGGAAGAAGCCACCAGAGATACTCACGATTCTGCACATACCTGGC
TAATCCCAGATCCTAAGGATTACATTAAGTTTACTAACATTTATATAATGATTTATAGTTTAAAGTATA
AACTTATCTAATTTACTATTCTGACAGATATTAATTAATCCTCAAATATCATAAGAGATGATTACTATT
ATCCCCATTTAACACAAGAGGAAACTGAGAGGGAAAGATGTTGAAGTAATTTTCCCACAATTACAGCAT
CCGTTAGTTACGACTCTATGATCTTCTGACACAAATTCCATTTACTCCTCACCCTATGACTCAGTCGAA
TATATCAAAGTTATGGACATTATGCTAAGTAACAAATTACCCTTTTATATAGTAAATACTGAGTAGATT
GAGAGAAGAAATTGTTTGCAAACCTGAATAGCTTCAAGAAGAAGAGAAGTGAGGATAAGAATAACAGTT
GTCATTTAACAAGTTTTAACAAGTAACTTGGTTAGAAAGGGATTCAAATGCATAAAGCAAGGGATAAAT
TTTTCTGGCAACAAGACTATACAATATAACCTTAAATATGACTTCAAATAATTGTTGGAACTTGATAAA
ACTAATTAAATATTATTGAAGATTATCAATATTATAAATGTAATTTACTTTTAAAAAGGGAACATAGAA
ATGTGTATCATTAGAGTAGAAAACAATCCTTATTATCACAATTTGTCAAAACAAGTTTGTTATTAACAC
AAGTAGAATACTGCATTCAATTAAGTTGACTGCAGATTTTGTGTTTTGTTAAAATTAGAAAGAGATAAC
AACAATTTGAATTATTGAAAGTAACATGTAAATAGTTCTACATACGTTCTTTTGACATCTTGTTCAATC
ATTGATCGAAGTTCTTTATCTTGGAAGAATTTGTTCCAAAGACTCTGAAATAAGGAAAACAATCTATTA
TATAGTCTCACACCTTTGTTTTACTTTTAGTGATTTCAATTTAATAATGTAAATGGTTAAAATTTATTC
TTCTCTGAGATCATTTCACATTGCAGATAGAAAACCTGAGACTGGGGTAATTTTTATTAAAATCTAATT
TAATCTCAGAAACACATCTTTATTCTAACATCAATTTTTCCAGTTTGATATTATCATATAAAGTCAGCC
TTCCTCATCTGCAGGTTCCACAACAAAAATCCAACCAACTGTGGATCAAAAATATTGGGAAAAAATTAA
AAATAGCAATACAACAATAAAAAAATACAAATCAGAAAAACAGCACAGTATAACAACTTTATTTAGCAT
TTACAATCTATTAGGTATTATAAGTAATCTAGAATTAATTCCGTGTATTCTATAGTGTCACCTAAATCG
TATGTGTATGATACATAAGGTTATGTATTAATTGTAGCCGCGTTCTAACGACAATATGTACAAGCCTAA
TTGTGTAGCATCTGGCTTACTGAAGCAGACCCTATCATCTCTCTCGTAAACTGCCGTCAGAGTCGGTTT
GGTTGGACGAACCTTCTGAGTTTCTGGTAACGCCGTCCCGCACCCGGAAATGGTCAGCGAACCAATCAG
CAGGGTCATCGCTAGCCAGATCCTCTACGCCGGACGCATCGTGGCCGGCATCACCGGCGCCACAGGTGC
GGTTGCTGGCGCCTATATCGCCGACATCACCGATGGGGAAGATCGGGCTCGCCACTTCGGGCTCATGAG
CGCTTGTTTCGGCGTGGGTATGGTGGCAGGCCCCGTGGCCGGGGGACTGTTGGGCGCCATCTCCTTGCA
TGCACCATTCCTTGCGGCGGCGGTGCTCAACGGCCTCAACCTACTACTGGGCTGCTTCCTAATGCAGGA
GTCGCATAAGGGAGAGCGTCGAATGGTGCACTCTCAGTACAATCTGCTCTGATGCCGCATAGTTAAGCC
AGCCCCGACACCCGCCAACACCCGCTGACGCGCCCTGACGGGCTTGTCTGCTCCCGGCATCCGCTTACA
GACAAGCTGTGACCGTCTCCGGGAGCTGCATGTGTCAGAGGTTTTCACCGTCATCACCGAAACGCGCGA
GACGAAAGGGCCTCGTGATACGCCTATTTTTATAGGTTAATGTCATGATAATAATGGTTTCTTAGACGT
CAGGTGGCACTTTTCGGGGAAATGTGCGCGGAACCCCTATTTGTTTATTTTTCTAAATACATTCAAATA
TGTATCCGCTCATGAGACAATAACCCTGATAAATGCTTCAATAATATTGAAAAAGGAAGAGTATGAGTA
TTCAACATTTCCGTGTCGCCCTTATTCCCTTTTTTGCGGCATTTTGCCTTCCTGTTTTTGCTCACCCAG
AAACGCTGGTGAAAGTAAAAGATGCTGAAGATCAGTTGGGTGCACGAGTGGGTTACATCGAACTGGATC
TCAACAGCGGTAAGATCCTTGAGAGTTTTCGCCCCGAAGAACGTTTTCCAATGATGAGCACTTTTAAAG
TTCTGCTATGTGGCGCGGTATTATCCCGTATTGACGCCGGGCAAGAGCAACTCGGTCGCCGCATACACT
ATTCTCAGAATGACTTGGTTGAGTACTCACCAGTCACAGAAAAGCATCTTACGGATGGCATGACAGTAA
GAGAATTATGCAGTGCTGCCATAACCATGAGTGATAACACTGCGGCCAACTTACTTCTGACAACGATCG
GAGGACCGAAGGAGCTAACCGCTTTTTTGCACAACATGGGGGATCATGTAACTCGCCTTGATCGTTGGG
AACCGGAGCTGAATGAAGCCATACCAAACGACGAGCGTGACACCACGATGCCTGTAGCAATGGCAACAA
CGTTGCGCAAACTATTAACTGGCGAACTACTTACTCTAGCTTCCCGGCAACAATTAATAGACTGGATGG
AGGCGGATAAAGTTGCAGGACCACTTCTGCGCTCGGCCCTTCCGGCTGGCTGGTTTATTGCTGATAAAT
CTGGAGCCGGTGAGCGTGGGTCTCGCGGTATCATTGCAGCACTGGGGCCAGATGGTAAGCCCTCCCGTA
TCGTAGTTATCTACACGACGGGGAGTCAGGCAACTATGGATGAACGAAATAGACAGATCGCTGAGATAG
GTGCCTCACTGATTAAGCATTGGTAACTGTCAGACCAAGTTTACTCATATATACTTTAGATTGATTTAA
AACTTCATTTTTAATTTAAAAGGATCTAGGTGAAGATCCTTTTTGATAATCTCATGACCAAAATCCCTT
AACGTGAGTTTTCGTTCCACTGAGCGTCAGACCCCGTAGAAAAGATCAAAGGATCTTCTTGAGATCCTT
TTTTTCTGCGCGTAATCTGCTGCTTGCAAACAAAAAAACCACCGCTACCAGCGGTGGTTTGTTTGCCGG
ATCAAGAGCTACCAACTCTTTTTCCGAAGGTAACTGGCTTCAGCAGAGCGCAGATACCAAATACTGTTC
TTCTAGTGTAGCCGTAGTTAGGCCACCACTTCAAGAACTCTGTAGCACCGCCTACATACCTCGCTCTGC
TAATCCTGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTGGAGCGAA
CGACCTACACCGAACTGAGATACCTACAGCGTGAGCTATGAGAAAGCGCCACGCTTCCCGAAGGGAGAA
AGGCGGACAGGTATCCGGTAAGCGGCAGGGTCGGAACAGGAGAGCGCACGAGGGAGCTTCCAGGGGGAA
ACGCCTGGTATCTTTATAGTCCTGTCGGGTTTCGCCACCTCTGACTTGAGCGTCGATTTTTGTGATGCT
CGTCAGGGGGGCGGAGCCTATGGAAAAACGCCAGCAACGCGGCCTTTTTACGGTTCCTGGCCTTTTGCT
GGCCTTTTGCTCACATGTTCTTTCCTGCGTTATCCCCTGATTCTGTGGATAACCGTATTACCGCCTTTG
AGTGAGCTGATACCGCTCGCCGCAGCCGAACGACCGAGCGCAGCGAGTCAGTGAGCGAGGAAGCGGAAG
AGCGCCCAATACGCAAACCGCCTCTCCCCGCGCGTTGGCCGATTCATTAATGCAGCTGTGGAATGTGTG
TCAGTTAGGGTGTGGAAAGTCCCCAGGCTCCCCAGCAGGCAGAAGTATGCAAAGCATGCATCTCAATTA
GTCAGCAACCAGGTGTGGAAAGTCCCCAGGCTCCCCAGCAGGCAGAAGTATGCAAAGCATGCATCTCAA
TTAGTCAGCAACCATAGTCCCGCCCCTAACTCCGCCCATCCCGCCCCTAACTCCGCCCAGTTCCGCCCA
TTCTCCGCCCCATGGCTGACTAATTTTTTTTATTTATGCAGAGGCCGAGGCCGCCTCGGCCTCTGAGCT
ATTCCAGAAGTAGTGAGGAGGCTTTTTTGGAGGCCTAGGCTTTTGCAAAAAGCTTGGACACAAGACAGG
CTTGCGAGATATGTTTGAGAATACCACTTTATCCCGCGTCAGGGAGAGGCAGTGCGTAAAAAGACGCGG
ACTCATGTGAAATACTGGTTTTTAGTGCGCCAGATCTCTATAATCTCGCGCAACCTATTTTCCCCTCGA
ACACTTTTTAAGCCGTAGATAAACAGGCTGGGACACTTCACATGAGCGAAAAATACATCGTCACCTGGG
ACATGTTGCAGATCCATGCACGTAAACTCGCAAGCCGACTGATGCCTTCTGAACAATGGAAAGGCATTA
TTGCCGTAAGCCGTGGCGGTCTGTACCGGGTGCGTTACTGGCGCGTGAACTGGGTATTCGTCATGTCGA
TACCGTTTGTATTTCCAGCTACGATCACGACAACCAGCGCGAGCTTAAAGTGCTGAAACGCGCAGAAGG
CGATGGCGAAGGCTTCATCGTTATTGATGACCTGGTGGATACCGGTGGTACTGCGGTTGCGATTCGTGA
AATGTATCCAAAAGCGCACTTTGTCACCATCTTCGCAAAACCGGCTGGTCGTCCGCTGGTTGATGACTA
TGTTGTTGATATCCCGCAAGATACCTGGATTGAACAGCCGTGGGATATGGGCGTCGTATTCGTCCCGCC
AATCTCCGGTCGCTAATCTTTTCAACGCCTGGCACTGCCGGGCGTTGTTCTTTTTAACTTCAGGCGGGT
TACAATAGTTTCCAGTAAGTATTCTGGAGGCTGCATCCATGACACAGGCAAACCTGAGCGAAACCCTGT
TCAAACCCCGCTTTAAACATCCTGAAACCTCGACGCTAGTCCGCCGCTTTAATCACGGCGCACAACCGC
CTGTGCAGTCGGCCCTTGATGGTAAAACCATCCCTCACTGGTATCGCATGATTAACCGTCTGATGTGGA
TCTGGCGCGGCATTGACCCACGCGAAATCCTCGACGTCCAGGCACGTATTGTGATGAGCGATGCCGAAC
GTACCGACGATGATTTATACGATACGGTGATTGGCTACCGTGGCGGCAACTGGATTTATGAGTGGGCCC
CGGATCTTTGTGAAGGAACCTTACTTCTGTGGTGTGACATAATTGGACAAACTACCTACAGAGATTTAA
AGCTCTAAGGTAAATATAAAATTTTTAAGTGTATAATGTGTTAAACTACTGATTCTAATTGTTTGTGTA
TTTTAGATTCCAACCTATGGAACTGATGAATGGGAGCAGTGGTGGAATGCCTTTAATGAGGAAAACCTG
TTTTGCTCAGAAGAAATGCCATCTAGTGATGATGAGGCTACTGCTGACTCTCAACATTCTACTCCTCCA
AAAAAGAAGAGAAAGGTAGAAGACCCCAAGGACTTTCCTTCAGAATTGCTAAGTTTTTTGAGTCATGCT
GTGTTTAGTAATAGAACTCTTGCTTGCTTTGCTATTTACACCACAAAGGAAAAAGCTGCACTGCTATAC
AAGAAAATTATGGAAAAATATTCTGTAACCTTTATAAGTAGGCATAACAGTTATAATCATAACATACTG
TTTTTTCTTACTCCACACAGGCATAGAGTGTCTGCTATTAATAACTATGCTCAAAAATTGTGTACCTTT
AGCTTTTTAATTTGTAAAGGGGTTAATAAGGAATATTTGATGTATAGTGCCTTGACTAGAGATCATAAT
CAGCCATACCACATTTGTAGAGGTTTTACTTGCTTTAAAAAACCTCCCACACCTCCCCCTGAACCTGAA
ACATAAAATGAATGCAATTGTTGTTGTTAACTTGTTTATTGCAGCTTATAATGGTTACAAATAAAGCAA
TAGCATCACAAATTTCACAAATAAAGCATTTTTTTCACTGCATTCTAGTTGTGGTTTGTCCAAACTCAT
CAATGTATCTTATCATGTCTGGATCAACTGGATAACTCAAGCTAACCAAAATCATCCCAAACTTCCCAC
CCCATACCCTATTACCACTGCC
pHR Backbone
AATTACCTGTGGTTTCATTTACTCTAAACCTGTGATTCCTCTGAATTATTTTCATTTTAAAGAAATTGT
ATTTGTTAAATATGTACTACAAACTTAGTAGTTGGAAGGGCTAATTCACTCCCAAAGAAGACAAGATAT
CCTTGATCTGTGGATCTACCACACACAAGGCTACTTCCCTGATTAGCAGAACTACACACCAGGGCCAGG
GGTCAGATATCCACTGACCTTTGGATGGTGCTACAAGCTAGTACCAGTTGAGCCAGATAAGGTAGAAGA
GGCCAATAAAGGAGAGAACACCAGCTTGTTACACCCTGTGAGCCTGCATGGGATGGATGACCCGGAGAG
AGAAGTGTTAGAGTGGAGGTTTGACAGCCGCCTAGCATTTCATCACGTGGCCCGAGAGCTGCATCCGGA
GTACTTCAAGAACTGCTGATATCGAGCTTGCTACAAGGGACTTTCCGCTGGGGACTTTCCAGGGAGGCG
TGGCCTGGGCGGGACTGGGGAGTGGCGAGCCCTCAGATCCTGCATATAAGCAGCTGCTTTTTGCCTGTA
CTGGGTCTCTCTGGTTAGACCAGATCTGAGCCTGGGAGCTCTCTGGCTAACTAGGGAACCCACTGCTTA
AGCCTCAATAAAGCTTGCCTTGAGTGCTTCAAGTAGTGTGTGCCCGTCTGTTGTGTGACTCTGGTAACT
AGAGATCCCTCAGACCCTTTTAGTCAGTGTGGAAAATCTCTAGCAGTGGCGCCCGAACAGGGACTTGAA
AGCGAAAGGGAAACCAGAGGAGCTCTCTCGACGCAGGACTCGGCTTGCTGAAGCGCGCACGGCAAGAGG
CGAGGGGCGGCGACTGGTGAGTACGCCAAAAATTTTGACTAGCGGAGGCTAGAAGGAGAGAGATGGGTG
CGAGAGCGTCAGTATTAAGCGGGGGAGAATTAGATCGCGATGGGAAAAAATTCGGTTAAGGCCAGGGGG
AAAGAAAAAATATAAATTAAAACATATAGTATGGGCAAGCAGGGAGCTAGAACGATTCGCAGTTAATCC
TGGCCTGTTAGAAACATCAGAAGGCTGTAGACAAATACTGGGACAGCTACAACCATCCCTTCAGACAGG
ATCAGAAGAACTTAGATCATTATATAATACAGTAGCAACCCTCTATTGTGTGCATCAAAGGATAGAGAT
AAAAGACACCAAGGAAGCTTTAGACAAGATAGAGGAAGAGCAAAACAAAAGTAAGACCACCGCACAGCA
AGCGGCCGCTGATCTTCAGACCTGGAGGAGGAGATATGAGGGACAATTGGAGAAGTGAATTATATAAAT
ATAAAGTAGTAAAAATTGAACCATTAGGAGTAGCACCCACCAAGGCAAAGAGAAGAGTGGTGCAGAGAG
AAAAAAGAGCAGTGGGAATAGGAGCTTTGTTCCTTGGGTTCTTGGGAGCAGCAGGAAGCACTATGGGCG
CAGCGTCAATGACGCTGACGGTACAGGCCAGACAATTATTGTCTGGTATAGTGCAGCAGCAGAACAATT
TGCTGAGGGCTATTGAGGCGCAACAGCATCTGTTGCAACTCACAGTCTGGGGCATCAAGCAGCTCCAGG
CAAGAATCCTGGCTGTGGAAAGATACCTAAAGGATCAACAGCTCCTGGGGATTTGGGGTTGCTCTGGAA
AACTCATTTGCACCACTGCTGTGCCTTGGAATGCTAGTTGGAGTAATAAATCTCTGGAACAGATTTGGA
ATCACACGACCTGGATGGAGTGGGACAGAGAAATTAACAATTACACAAGCTTAATACACTCCTTAATTG
AAGAATCGCAAAACCAGCAAGAAAAGAATGAACAAGAATTATTGGAATTAGATAAATGGGCAAGTTTGT
GGAATTGGTTTAACATAACAAATTGGCTGTGGTATATAAAATTATTCATAATGATAGTAGGAGGCTTGG
TAGGTTTAAGAATAGTTTTTGCTGTACTTTCTATAGTGAATAGAGTTAGGCAGGGATATTCACCATTAT
CGTTTCAGACCCACCTCCCAACCCCGAGGGGACCCGACAGGCCCGAAGGAATAGAAGAAGAAGGTGGAG
AGAGAGACAGAGACAGATCCATTCGATTAGTGAACGGATCTCGACGGTATCGATTTTAAAAGAAAAGGG
GGGATTGGGGGGTACAGTGCAGGGGAAAGAATAGTAGACATAATAGCAACAGACATACAAACTAAAGAA
TTACAAAAACAAATTACAAAAATTCAAAATTTTATCGATAAGCTTTGCAAAGATGGATAAAGTTTTAAA
CAGAGAGGAATCTTTGCAGCTAATGGACCTTCTAGGTCTTGAAAGGAGTGGGAATTGGCTCCGGTGCCC
GTCAGTGGGCAGAGCGCACATCGCCCACAGTCCCCGAGAAGTTGGGGGGAGGGGTCGGCAATTGAACCG
GTGCCTAGAGAAGGTGGCGCGGGGTAAACTGGGAAAGTGATGTCGTGTACTGGCTCCGCCTTTTTCCCG
AGGGTGGGGGAGAACCGTATATAAGTGCAGTAGTCGCCGTGAACGTTCTTTTTCGCAACGGGTTTGCCG
CCAGAACACAGGTAAGTGCCGTGTGTGGTTCCCGCGGGCCTGGCCTCTTTACGGGTTATGGCCCTTGCG
TGCCTTGAATTACTTCCACCTGGCTGCAGTACGTGATTCTTGATCCCGAGCTTCGGGTTGGAAGTGGGT
GGGAGAGTTCGAGGCCTTGCGCTTAAGGAGCCCCTTCGCCTCGTGCTTGAGTTGAGGCCTGGCCTGGGC
GCTGGGGCCGCCGCGTGCGAATCTGGTGGCACCTTCGCGCCTGTCTCGCTGCTTTCGATAAGTCTCTAG
CCATTTAAAATTTTTGATGACCTGCTGCGACGCTTTTTTTCTGGCAAGATAGTCTTGTAAATGCGGGCC
AAGATCTGCACACTGGTATTTCGGTTTTTGGGGCCGCGGGCGGCGACGGGGCCCGTGCGTCCCAGCGCA
CATGTTCGGCGAGGCGGGGCCTGCGAGCGCGGCCACCGAGAATCGGACGGGGGTAGTCTCAAGCTGGCC
GGCCTGCTCTGGTGCCTGGCCTCGCGCCGCCGTGTATCGCCCCGCCCTGGGCGGCAAGGCTGGCCCGGT
CGGCACCAGTTGCGTGAGCGGAAAGATGGCCGCTTCCCGGCCCTGCTGCAGGGAGCTCAAAATGGAGGA
CGCGGCGCTCGGGAGAGCGGGCGGGTGAGTCACCCACACAAAGGAAAAGGGCCTTTCCGTCCTCAGCCG
TCGCTTCATGTGACTCCACGGAGTACCGGGCGCCGTCCAGGCACCTCGATTAGTTCTCGAGCTTTTGGA
GTACGTCGTCTTTAGGTTGGGGGGAGGGGTTTTATGCGATGGAGTTTCCCCACACTGAGTGGGTGGAGA
CTGAAGTTAGGCCAGCTTGGCACTTGATGTAATTCTCCTTGGAATTTGCCCTTTTTGAGTTTGGATCTT
GGTTCATTCTCAAGCCTCAGACAGTGGTTCAAAGTTTTTTTCTTCCATTTCAGGTGTCGTGAG
AATTACCTGTGGTTTCATTTACTCTAAACCTGTGATTCCTCTGAATTATTTTCATTTTAAAGAAATTGT
ATTTGTTAAATATGTACTACAAACTTAGTAGTTGGAAGGGCTAATTCACTCCCAAAGAAGACAAGATAT
CCTTGATCTGTGGATCTACCACACACAAGGCTACTTCCCTGATTAGCAGAACTACACACCAGGGCCAGG
GGTCAGATATCCACTGACCTTTGGATGGTGCTACAAGCTAGTACCAGTTGAGCCAGATAAGGTAGAAGA
GGCCAATAAAGGAGAGAACACCAGCTTGTTACACCCTGTGAGCCTGCATGGGATGGATGACCCGGAGAG
AGAAGTGTTAGAGTGGAGGTTTGACAGCCGCCTAGCATTTCATCACGTGGCCCGAGAGCTGCATCCGGA
GTACTTCAAGAACTGCTGATATCGAGCTTGCTACAAGGGACTTTCCGCTGGGGACTTTCCAGGGAGGCG
TGGCCTGGGCGGGACTGGGGAGTGGCGAGCCCTCAGATCCTGCATATAAGCAGCTGCTTTTTGCCTGTA
CTGGGTCTCTCTGGTTAGACCAGATCTGAGCCTGGGAGCTCTCTGGCTAACTAGGGAACCCACTGCTTA
AGCCTCAATAAAGCTTGCCTTGAGTGCTTCAAGTAGTGTGTGCCCGTCTGTTGTGTGACTCTGGTAACT
AGAGATCCCTCAGACCCTTTTAGTCAGTGTGGAAAATCTCTAGCAGTGGCGCCCGAACAGGGACTTGAA
AGCGAAAGGGAAACCAGAGGAGCTCTCTCGACGCAGGACTCGGCTTGCTGAAGCGCGCACGGCAAGAGG
CGAGGGGCGGCGACTGGTGAGTACGCCAAAAATTTTGACTAGCGGAGGCTAGAAGGAGAGAGATGGGTG
CGAGAGCGTCAGTATTAAGCGGGGGAGAATTAGATCGCGATGGGAAAAAATTCGGTTAAGGCCAGGGGG
AAAGAAAAAATATAAATTAAAACATATAGTATGGGCAAGCAGGGAGCTAGAACGATTCGCAGTTAATCC
TGGCCTGTTAGAAACATCAGAAGGCTGTAGACAAATACTGGGACAGCTACAACCATCCCTTCAGACAGG
ATCAGAAGAACTTAGATCATTATATAATACAGTAGCAACCCTCTATTGTGTGCATCAAAGGATAGAGAT
AAAAGACACCAAGGAAGCTTTAGACAAGATAGAGGAAGAGCAAAACAAAAGTAAGACCACCGCACAGCA
AGCGGCCGCTGATCTTCAGACCTGGAGGAGGAGATATGAGGGACAATTGGAGAAGTGAATTATATAAAT
ATAAAGTAGTAAAAATTGAACCATTAGGAGTAGCACCCACCAAGGCAAAGAGAAGAGTGGTGCAGAGAG
AAAAAAGAGCAGTGGGAATAGGAGCTTTGTTCCTTGGGTTCTTGGGAGCAGCAGGAAGCACTATGGGCG
CAGCGTCAATGACGCTGACGGTACAGGCCAGACAATTATTGTCTGGTATAGTGCAGCAGCAGAACAATT
TGCTGAGGGCTATTGAGGCGCAACAGCATCTGTTGCAACTCACAGTCTGGGGCATCAAGCAGCTCCAGG
CAAGAATCCTGGCTGTGGAAAGATACCTAAAGGATCAACAGCTCCTGGGGATTTGGGGTTGCTCTGGAA
AACTCATTTGCACCACTGCTGTGCCTTGGAATGCTAGTTGGAGTAATAAATCTCTGGAACAGATTTGGA
ATCACACGACCTGGATGGAGTGGGACAGAGAAATTAACAATTACACAAGCTTAATACACTCCTTAATTG
AAGAATCGCAAAACCAGCAAGAAAAGAATGAACAAGAATTATTGGAATTAGATAAATGGGCAAGTTTGT
GGAATTGGTTTAACATAACAAATTGGCTGTGGTATATAAAATTATTCATAATGATAGTAGGAGGCTTGG
TAGGTTTAAGAATAGTTTTTGCTGTACTTTCTATAGTGAATAGAGTTAGGCAGGGATATTCACCATTAT
CGTTTCAGACCCACCTCCCAACCCCGAGGGGACCCGACAGGCCCGAAGGAATAGAAGAAGAAGGTGGAG
AGAGAGACAGAGACAGATCCATTCGATTAGTGAACGGATCTCGACGGTATCGATTTTAAAAGAAAAGGG
GGGATTGGGGGGTACAGTGCAGGGGAAAGAATAGTAGACATAATAGCAACAGACATACAAACTAAAGAA
TTACAAAAACAAATTACAAAAATTCAAAATTTTATCGATAAGCTTTGCAAAGATGGATAAAGTTTTAAA
CAGAGAGGAATCTTTGCAGCTAATGGACCTTCTAGGTCTTGAAAGGAGTGGGAATTGGCTCCGGTGCCC
GTCAGTGGGCAGAGCGCACATCGCCCACAGTCCCCGAGAAGTTGGGGGGAGGGGTCGGCAATTGAACCG
GTGCCTAGAGAAGGTGGCGCGGGGTAAACTGGGAAAGTGATGTCGTGTACTGGCTCCGCCTTTTTCCCG
AGGGTGGGGGAGAACCGTATATAAGTGCAGTAGTCGCCGTGAACGTTCTTTTTCGCAACGGGTTTGCCG
CCAGAACACAGGTAAGTGCCGTGTGTGGTTCCCGCGGGCCTGGCCTCTTTACGGGTTATGGCCCTTGCG
TGCCTTGAATTACTTCCACCTGGCTGCAGTACGTGATTCTTGATCCCGAGCTTCGGGTTGGAAGTGGGT
GGGAGAGTTCGAGGCCTTGCGCTTAAGGAGCCCCTTCGCCTCGTGCTTGAGTTGAGGCCTGGCCTGGGC
GCTGGGGCCGCCGCGTGCGAATCTGGTGGCACCTTCGCGCCTGTCTCGCTGCTTTCGATAAGTCTCTAG
CCATTTAAAATTTTTGATGACCTGCTGCGACGCTTTTTTTCTGGCAAGATAGTCTTGTAAATGCGGGCC
AAGATCTGCACACTGGTATTTCGGTTTTTGGGGCCGCGGGCGGCGACGGGGCCCGTGCGTCCCAGCGCA
CATGTTCGGCGAGGCGGGGCCTGCGAGCGCGGCCACCGAGAATCGGACGGGGGTAGTCTCAAGCTGGCC
GGCCTGCTCTGGTGCCTGGCCTCGCGCCGCCGTGTATCGCCCCGCCCTGGGCGGCAAGGCTGGCCCGGT
CGGCACCAGTTGCGTGAGCGGAAAGATGGCCGCTTCCCGGCCCTGCTGCAGGGAGCTCAAAATGGAGGA
CGCGGCGCTCGGGAGAGCGGGCGGGTGAGTCACCCACACAAAGGAAAAGGGCCTTTCCGTCCTCAGCCG
TCGCTTCATGTGACTCCACGGAGTACCGGGCGCCGTCCAGGCACCTCGATTAGTTCTCGAGCTTTTGGA
GTACGTCGTCTTTAGGTTGGGGGGAGGGGTTTTATGCGATGGAGTTTCCCCACACTGAGTGGGTGGAGA
CTGAAGTTAGGCCAGCTTGGCACTTGATGTAATTCTCCTTGGAATTTGCCCTTTTTGAGTTTGGATCTT
GGTTCATTCTCAAGCCTCAGACAGTGGTTCAAAGTTTTTTTCTTCCATTTCAGGTGTCGTGAGGAATTC
GGATCCAAGCTTCAATTGTGGTCACTCGACAATCAACCTCTGGATTACAAAATTTGTGAAAGATTGACT
GGTATTCTTAACTATGTTGCTCCTTTTACGCTATGTGGATACGCTGCTTTAATGCCTTTGTATCATGCT
ATTGCTTCCCGTATGGCTTTCATTTTCTCCTCCTTGTATAAATCCTGGTTGCTGTCTCTTTATGAGGAG
TTGTGGCCCGTTGTCAGGCAACGTGGCGTGGTGTGCACTGTGTTTGCTGACGCAACCCCCACTGGTTGG
GGCATTGCCACCACCTGTCAGCTCCTTTCCGGGACTTTCGCTTTCCCCCTCCCTATTGCCACGGCGGAA
CTCATCGCCGCCTGCCTTGCCCGCTGCTGGACAGGGGCTCGGCTGTTGGGCACTGACAATTCCGTGGTG
TTGTCGGGGAAGCTGACGTCCTTTCCATGGCTGCTCGCCTGTGTTGCCACCTGGATTCTGCGCGGGACG
TCCTTCTGCTACGTCCCTTCGGCCCTCAATCCAGCGGACCTTCCTTCCCGCGGCCTGCTGCCGGCTCTG
CGGCCTCTTCCGCGTCTTCGCCTTCGCCCTCAGACGAGTCGGATCTCCCTTTGGGCCGCCTCCCCGCCT
GCTCGAGACCTAGAAAAACATGGAGCAATCACAAGTAGCAATACAGCAGCTACCAATGCTGATTGTGCC
TGGCTAGAAGCACAAGAGGAGGAGGAGGTGGGTTTTCCAGTCACACCTCAGGTACCTTTAAGACCAATG
ACTTACAAGGCAGCTGTAGATCTTAGCCACTTTTTAAAAGAAAAGGGGGGACTGGAAGGGCTAATTCAC
TCCCAACGAAGACAAGATCTGCTTTTTGCTTGTACTGGGTCTCTCTGGTTAGACCAGATCTGAGCCTGG
GAGCTCTCTGGCTAACTAGGGAACCCACTGCTTAAGCCTCAATAAAGCTTGCCTTGAGTGCTTCAAGTA
GTGTGTGCCCGTCTGTTGTGTGACTCTGGTAACTAGAGATCCCTCAGACCCTTTTAGTCAGTGTGGAAA
ATCTCTAGCAGTAGTAGTTCATGTCATCTTATTATTCAGTATTTATAACTTGCAAAGAAATGAATATCA
GAGAGTGAGAGGCCTTGACATTATAATAGATTTAGCAGGAATTGAACTAGGAGTGGAGCACACAGGCAA
AGCTGCAGAAGTACTTGGAAGAAGCCACCAGAGATACTCACGATTCTGCACATACCTGGCTAATCCCAG
ATCCTAAGGATTACATTAAGTTTACTAACATTTATATAATGATTTATAGTTTAAAGTATAAACTTATCT
AATTTACTATTCTGACAGATATTAATTAATCCTCAAATATCATAAGAGATGATTACTATTATCCCCATT
TAACACAAGAGGAAACTGAGAGGGAAAGATGTTGAAGTAATTTTCCCACAATTACAGCATCCGTTAGTT
ACGACTCTATGATCTTCTGACACAAATTCCATTTACTCCTCACCCTATGACTCAGTCGAATATATCAAA
GTTATGGACATTATGCTAAGTAACAAATTACCCTTTTATATAGTAAATACTGAGTAGATTGAGAGAAGA
AATTGTTTGCAAACCTGAATAGCTTCAAGAAGAAGAGAAGTGAGGATAAGAATAACAGTTGTCATTTAA
CAAGTTTTAACAAGTAACTTGGTTAGAAAGGGATTCAAATGCATAAAGCAAGGGATAAATTTTTCTGGC
AACAAGACTATACAATATAACCTTAAATATGACTTCAAATAATTGTTGGAACTTGATAAAACTAATTAA
ATATTATTGAAGATTATCAATATTATAAATGTAATTTACTTTTAAAAAGGGAACATAGAAATGTGTATC
ATTAGAGTAGAAAACAATCCTTATTATCACAATTTGTCAAAACAAGTTTGTTATTAACACAAGTAGAAT
ACTGCATTCAATTAAGTTGACTGCAGATTTTGTGTTTTGTTAAAATTAGAAAGAGATAACAACAATTTG
AATTATTGAAAGTAACATGTAAATAGTTCTACATACGTTCTTTTGACATCTTGTTCAATCATTGATCGA
AGTTCTTTATCTTGGAAGAATTTGTTCCAAAGACTCTGAAATAAGGAAAACAATCTATTATATAGTCTC
ACACCTTTGTTTTACTTTTAGTGATTTCAATTTAATAATGTAAATGGTTAAAATTTATTCTTCTCTGAG
ATCATTTCACATTGCAGATAGAAAACCTGAGACTGGGGTAATTTTTATTAAAATCTAATTTAATCTCAG
AAACACATCTTTATTCTAACATCAATTTTTCCAGTTTGATATTATCATATAAAGTCAGCCTTCCTCATC
TGCAGGTTCCACAACAAAAATCCAACCAACTGTGGATCAAAAATATTGGGAAAAAATTAAAAATAGCAA
TACAACAATAAAAAAATACAAATCAGAAAAACAGCACAGTATAACAACTTTATTTAGCATTTACAATCT
ATTAGGTATTATAAGTAATCTAGAATTAATTCCGTGTATTCTATAGTGTCACCTAAATCGTATGTGTAT
GATACATAAGGTTATGTATTAATTGTAGCCGCGTTCTAACGACAATATGTACAAGCCTAATTGTGTAGC
ATCTGGCTTACTGAAGCAGACCCTATCATCTCTCTCGTAAACTGCCGTCAGAGTCGGTTTGGTTGGACG
AACCTTCTGAGTTTCTGGTAACGCCGTCCCGCACCCGGAAATGGTCAGCGAACCAATCAGCAGGGTCAT
CGCTAGCCAGATCCTCTACGCCGGACGCATCGTGGCCGGCATCACCGGCGCCACAGGTGCGGTTGCTGG
CGCCTATATCGCCGACATCACCGATGGGGAAGATCGGGCTCGCCACTTCGGGCTCATGAGCGCTTGTTT
CGGCGTGGGTATGGTGGCAGGCCCCGTGGCCGGGGGACTGTTGGGCGCCATCTCCTTGCATGCACCATT
CCTTGCGGCGGCGGTGCTCAACGGCCTCAACCTACTACTGGGCTGCTTCCTAATGCAGGAGTCGCATAA
GGGAGAGCGTCGAATGGTGCACTCTCAGTACAATCTGCTCTGATGCCGCATAGTTAAGCCAGCCCCGAC
ACCCGCCAACACCCGCTGACGCGCCCTGACGGGCTTGTCTGCTCCCGGCATCCGCTTACAGACAAGCTG
TGACCGTCTCCGGGAGCTGCATGTGTCAGAGGTTTTCACCGTCATCACCGAAACGCGCGAGACGAAAGG
GCCTCGTGATACGCCTATTTTTATAGGTTAATGTCATGATAATAATGGTTTCTTAGACGTCAGGTGGCA
CTTTTCGGGGAAATGTGCGCGGAACCCCTATTTGTTTATTTTTCTAAATACATTCAAATATGTATCCGC
TCATGAGACAATAACCCTGATAAATGCTTCAATAATATTGAAAAAGGAAGAGTATGAGTATTCAACATT
TCCGTGTCGCCCTTATTCCCTTTTTTGCGGCATTTTGCCTTCCTGTTTTTGCTCACCCAGAAACGCTGG
TGAAAGTAAAAGATGCTGAAGATCAGTTGGGTGCACGAGTGGGTTACATCGAACTGGATCTCAACAGCG
GTAAGATCCTTGAGAGTTTTCGCCCCGAAGAACGTTTTCCAATGATGAGCACTTTTAAAGTTCTGCTAT
GTGGCGCGGTATTATCCCGTATTGACGCCGGGCAAGAGCAACTCGGTCGCCGCATACACTATTCTCAGA
ATGACTTGGTTGAGTACTCACCAGTCACAGAAAAGCATCTTACGGATGGCATGACAGTAAGAGAATTAT
GCAGTGCTGCCATAACCATGAGTGATAACACTGCGGCCAACTTACTTCTGACAACGATCGGAGGACCGA
AGGAGCTAACCGCTTTTTTGCACAACATGGGGGATCATGTAACTCGCCTTGATCGTTGGGAACCGGAGC
TGAATGAAGCCATACCAAACGACGAGCGTGACACCACGATGCCTGTAGCAATGGCAACAACGTTGCGCA
AACTATTAACTGGCGAACTACTTACTCTAGCTTCCCGGCAACAATTAATAGACTGGATGGAGGCGGATA
AAGTTGCAGGACCACTTCTGCGCTCGGCCCTTCCGGCTGGCTGGTTTATTGCTGATAAATCTGGAGCCG
GTGAGCGTGGGTCTCGCGGTATCATTGCAGCACTGGGGCCAGATGGTAAGCCCTCCCGTATCGTAGTTA
TCTACACGACGGGGAGTCAGGCAACTATGGATGAACGAAATAGACAGATCGCTGAGATAGGTGCCTCAC
TGATTAAGCATTGGTAACTGTCAGACCAAGTTTACTCATATATACTTTAGATTGATTTAAAACTTCATT
TTTAATTTAAAAGGATCTAGGTGAAGATCCTTTTTGATAATCTCATGACCAAAATCCCTTAACGTGAGT
TTTCGTTCCACTGAGCGTCAGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTTTGTTTGCCGGATCAAGAGC
TACCAACTCTTTTTCCGAAGGTAACTGGCTTCAGCAGAGCGCAGATACCAAATACTGTTCTTCTAGTGT
AGCCGTAGTTAGGCCACCACTTCAAGAACTCTGTAGCACCGCCTACATACCTCGCTCTGCTAATCCTGT
TACCAGTGGCTGCTGCCAGTGGCGATAAGTCGTGTCTTACCGGGTTGGACTCAAGACGATAGTTACCGG
ATAAGGCGCAGCGGTCGGGCTGAACGGGGGGTTCGTGCACACAGCCCAGCTTGGAGCGAACGACCTACA
CCGAACTGAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAACGCCTGGT
ATCTTTATAGTCCTGTCGGGTTTCGCCACCTCTGACTTGAGCGTCGATTTTTGTGATGCTCGTCAGGGG
GGCGGAGCCTATGGAAAAACGCCAGCAACGCGGCCTTTTTACGGTTCCTGGCCTTTTGCTGGCCTTTTG
CTCACATGTTCTTTCCTGCGTTATCCCCTGATTCTGTGGATAACCGTATTACCGCCTTTGAGTGAGCTG
ATACCGCTCGCCGCAGCCGAACGACCGAGCGCAGCGAGTCAGTGAGCGAGGAAGCGGAAGAGCGCCCAA
TACGCAAACCGCCTCTCCCCGCGCGTTGGCCGATTCATTAATGCAGCTGTGGAATGTGTGTCAGTTAGG
GTGTGGAAAGTCCCCAGGCTCCCCAGCAGGCAGAAGTATGCAAAGCATGCATCTCAATTAGTCAGCAAC
CAGGTGTGGAAAGTCCCCAGGCTCCCCAGCAGGCAGAAGTATGCAAAGCATGCATCTCAATTAGTCAGC
AACCATAGTCCCGCCCCTAACTCCGCCCATCCCGCCCCTAACTCCGCCCAGTTCCGCCCATTCTCCGCC
CCATGGCTGACTAATTTTTTTTATTTATGCAGAGGCCGAGGCCGCCTCGGCCTCTGAGCTATTCCAGAA
GTAGTGAGGAGGCTTTTTTGGAGGCCTAGGCTTTTGCAAAAAGCTTGGACACAAGACAGGCTTGCGAGA
TATGTTTGAGAATACCACTTTATCCCGCGTCAGGGAGAGGCAGTGCGTAAAAAGACGCGGACTCATGTG
AAATACTGGTTTTTAGTGCGCCAGATCTCTATAATCTCGCGCAACCTATTTTCCCCTCGAACACTTTTT
AAGCCGTAGATAAACAGGCTGGGACACTTCACATGAGCGAAAAATACATCGTCACCTGGGACATGTTGC
AGATCCATGCACGTAAACTCGCAAGCCGACTGATGCCTTCTGAACAATGGAAAGGCATTATTGCCGTAA
GCCGTGGCGGTCTGTACCGGGTGCGTTACTGGCGCGTGAACTGGGTATTCGTCATGTCGATACCGTTTG
TATTTCCAGCTACGATCACGACAACCAGCGCGAGCTTAAAGTGCTGAAACGCGCAGAAGGCGATGGCGA
AGGCTTCATCGTTATTGATGACCTGGTGGATACCGGTGGTACTGCGGTTGCGATTCGTGAAATGTATCC
AAAAGCGCACTTTGTCACCATCTTCGCAAAACCGGCTGGTCGTCCGCTGGTTGATGACTATGTTGTTGA
TATCCCGCAAGATACCTGGATTGAACAGCCGTGGGATATGGGCGTCGTATTCGTCCCGCCAATCTCCGG
TCGCTAATCTTTTCAACGCCTGGCACTGCCGGGCGTTGTTCTTTTTAACTTCAGGCGGGTTACAATAGT
TTCCAGTAAGTATTCTGGAGGCTGCATCCATGACACAGGCAAACCTGAGCGAAACCCTGTTCAAACCCC
GCTTTAAACATCCTGAAACCTCGACGCTAGTCCGCCGCTTTAATCACGGCGCACAACCGCCTGTGCAGT
CGGCCCTTGATGGTAAAACCATCCCTCACTGGTATCGCATGATTAACCGTCTGATGTGGATCTGGCGCG
GCATTGACCCACGCGAAATCCTCGACGTCCAGGCACGTATTGTGATGAGCGATGCCGAACGTACCGACG
ATGATTTATACGATACGGTGATTGGCTACCGTGGCGGCAACTGGATTTATGAGTGGGCCCCGGATCTTT
GTGAAGGAACCTTACTTCTGTGGTGTGACATAATTGGACAAACTACCTACAGAGATTTAAAGCTCTAAG
GTAAATATAAAATTTTTAAGTGTATAATGTGTTAAACTACTGATTCTAATTGTTTGTGTATTTTAGATT
CCAACCTATGGAACTGATGAATGGGAGCAGTGGTGGAATGCCTTTAATGAGGAAAACCTGTTTTGCTCA
GAAGAAATGCCATCTAGTGATGATGAGGCTACTGCTGACTCTCAACATTCTACTCCTCCAAAAAAGAAG
AGAAAGGTAGAAGACCCCAAGGACTTTCCTTCAGAATTGCTAAGTTTTTTGAGTCATGCTGTGTTTAGT
AATAGAACTCTTGCTTGCTTTGCTATTTACACCACAAAGGAAAAAGCTGCACTGCTATACAAGAAAATT
ATGGAAAAATATTCTGTAACCTTTATAAGTAGGCATAACAGTTATAATCATAACATACTGTTTTTTCTT
ACTCCACACAGGCATAGAGTGTCTGCTATTAATAACTATGCTCAAAAATTGTGTACCTTTAGCTTTTTA
ATTTGTAAAGGGGTTAATAAGGAATATTTGATGTATAGTGCCTTGACTAGAGATCATAATCAGCCATAC
CACATTTGTAGAGGTTTTACTTGCTTTAAAAAACCTCCCACACCTCCCCCTGAACCTGAAACATAAAAT
GAATGCAATTGTTGTTGTTAACTTGTTTATTGCAGCTTATAATGGTTACAAATAAAGCAATAGCATCAC
AAATTTCACAAATAAAGCATTTTTTTCACTGCATTCTAGTTGTGGTTTGTCCAAACTCATCAATGTATC
TTATCATGTCTGGATCAACTGGATAACTCAAGCTAACCAAAATCATCCCAAACTTCCCACCCCATACCC
TATTACCACTGCC
tmpk sequences
<211> 639
<212> DNA
<213> Homo sapiens

| | |
|---|---|
| atggcggccc ggcgcggggc tctcatagtg ctggagggcg tggaccgcgc cgggaagagc | 60 |
| acgcagagcc gcaagctggt ggaagcgctg tgcgccgcgg gccaccgcgc cgaactgctc | 120 |
| cggttcccgg aaagatcaac tgaaatcggc aaacttctga gttcctactt gcaaaagaaa | 180 |
| agtgacgtgg aggatcactc ggtgcacctg cttttttctg caaatcgctg ggaacaagtg | 240 |
| ccgttaatta aggaaaagtt gagccagggc gtgaccctcg tcgtggacag atacgcattt | 300 |
| tctggtgtgg ccttcaccgg tgccaaggag aatttttccc tagattggtg taaacagcca | 360 |
| gacgtgggcc ttcccaaacc cgacctggtc ctgttcctcc agttacagct ggcggatgct | 420 |
| gccaagcggg gagcgtttgg ccatgagcgc tatgagaacg gggctttcca ggagcgggcg | 480 |
| ctccggtgtt tccaccagct catgaaagac acgactttga actggaagat ggtggatgct | 540 |
| tccaaaagca tcgaagctgt ccatgaggac atccgcgtgc tctctgagga cgccatccgc | 600 |
| actgccacag agaagccgct gggggagcta tggaagtga | 639 |

```
<212> PRT
<213> Homo sapiens
Met Ala Ala Arg Arg Gly Ala Leu Ile Val Leu Glu Gly Val Asp Arg
1 5 10 15
Ala Gly Lys Ser Thr Gln Ser Arg Lys Leu Val Glu Ala Leu Cys Ala
                        20 25 30
Ala Gly His Arg Ala Glu Leu Leu Arg Phe Pro Glu Arg Ser Thr Glu
                35 40 45
Ile Gly Lys Leu Leu Ser Ser Tyr Leu Gln Lys Lys Ser Asp Val Glu
        50 55 60
Asp His Ser Val His Leu Leu Phe Ser Ala Asn Arg Trp Glu Gln Val
65 70 75 80
Pro Leu Ile Lys Glu Lys Leu Ser Gln Gly Val Thr Leu Val Val Asp
                                85 90 95
Arg Tyr Ala Phe Ser Gly Val Ala Phe Thr Gly Ala Lys Glu Asn Phe
                        100 105 110
Ser Leu Asp Trp Cys Lys Gln Pro Asp Val Gly Leu Pro Lys Pro Asp
                115 120 125
Leu Val Leu Phe Leu Gln Leu Gln Leu Ala Asp Ala Ala Lys Arg Gly
        130 135 140
Ala Phe Gly His Glu Arg Tyr Glu Asn Gly Ala Phe Gln Glu Arg Ala
145 150 155 160
Leu Arg Cys Phe His Gln Leu Met Lys Asp Thr Thr Leu Asn Trp Lys
                                165 170 175
Met Val Asp Ala Ser Lys Ser Ile Glu Ala Val His Glu Asp Ile Arg
                        180 185 190
Val Leu Ser Glu Asp Ala Ile Arg Thr Ala Thr Glu Lys Pro Leu Gly
                195 200 205
Glu Leu Trp Lys
        210
<211> 639
<212> DNA
<213> Homo sapiens
```

| | |
|---|---|
| atggcggccc ggcgcggggc tctcatagtg ctggagggcg tggaccgcgc cgggaagagc | 60 |
| acgcagagcc gcaagctggt ggaagcgctg tgcgccgcgg gccaccgcgc cgaactgctc | 120 |
| cggttcccgg aaagatcaac tgaaatcggc aaacttctga gttcctactt gcaaaagaaa | 180 |
| agtgacgtgg aggatcactc ggtgcacctg cttttttctg caaatcgctg ggaacaagtg | 240 |
| ccgttaatta aggaaaagtt gagccagggc gtgaccctcg tcgtggacag atacgcattt | 300 |
| tctggtgtgg ccttcaccgg tgccaaggag aatttttccc tagattggtg taaacagcca | 360 |
| gacgtgggcc ttcccaaacc cgacctggtc ctgttcctcc agttacagct ggcggatgct | 420 |
| gccaagcggg gagcgtttgg ccatgagcgc tatgagaacg gggctttcca ggagcgggcg | 480 |
| ctccggtgtt tccaccagct catgaaagac acgactttga actggaagat ggtggatgct | 540 |
| tccaaaagca tcgaagctgt ccatgaggac atccgcgtgc tctctgagga cgccatccgc | 600 |
| actgccacag agaagccgct gggggagcta tggaagtga | 639 |

```
<211> 212
<212> PRT
<213> Homo sapiens
Met Ala Ala Arg Arg Gly Ala Leu Ile Val Leu Glu Gly Val Asp Arg
1 5 10 15
Ala Gly Lys Ser Thr Gln Ser Arg Lys Leu Val Glu Ala Leu Cys Ala
                        20 25 30
Ala Gly His Arg Ala Glu Leu Leu Arg Phe Pro Glu Arg Ser Thr Glu
                35 40 45
Ile Gly Lys Leu Leu Ser Ser Tyr Leu Gln Lys Lys Ser Asp Val Glu
        50 55 60
Asp His Ser Val His Leu Leu Phe Ser Ala Asn Arg Trp Glu Gln Val
65 70 75 80
Pro Leu Ile Lys Glu Lys Leu Ser Gln Gly Val Thr Leu Val Val Asp
                                85 90 95
Arg Tyr Ala Phe Ser Gly Val Ala Phe Thr Gly Ala Lys Glu Asn Phe
                        100 105 110
Ser Leu Asp Trp Cys Lys Gln Pro Asp Val Gly Leu Pro Lys Pro Asp
                115 120 125
Leu Val Leu Phe Leu Gln Leu Gln Leu Ala Asp Ala Ala Lys Arg Gly
        130 135 140
Ala Phe Gly His Glu Arg Tyr Glu Asn Gly Ala Phe Gln Glu Arg Ala
145 150 155 160
Leu Arg Cys Phe His GlnLeu Met Lys Asp Thr Thr Leu Asn Trp Lys
                                165 170 175
Met Val Asp Ala Ser LysSer Ile Glu Ala Val His Glu Asp Ile Arg
                        180 185 190
Val Leu Ser Glu Asp Ala Ile Arg Thr Ala Thr Glu Lys Pro LeuGly
                195 200 205
Glu Leu Trp Lys
        210
<211> 636
<212> DNA
<213> Homo sapiens
```

| | |
|---|---|
| atggcggccc ggcgcggggc tctcatagtg ctggagggcg tggaccgcgc cgggaagagc | 60 |
| acgcagagcc gcaagctggt ggaagcgctg tcgcgcgggc caccgcccga actgctccgg | 120 |
| ttcccggaaa gatcaactga aatcggcaaa cttctgagtt cctacttgca aaagaaaagt | 180 |
| gacgtggagg atcactcggt gcacctgctt ttttctgcaa atcgctggga acaagtgccg | 240 |
| ttaattaagg aaaagttgag ccagggcgtg accctcgtcg tggacagata cgcattttct | 300 |
| ggtgtggcct tcaccggtgc caaggagaat ttttccctag actggtgtaa acagccagac | 360 |
| gtgggccttc ccaaacccga cctggtcctg ttcctccagt tacagctggc ggatgctgcc | 420 |
| aagcggggag cgtttggcca tgagcgctat gagaacgggg ctttccagga gcgggcgctc | 480 |
| cggtgtttcc accagctcat gaaagacacg actttgaact ggaagatggt ggatgcttcc | 540 |
| aaaagactcg aagctgtcca tgaggaactc cgcgtgctct ctgaggacgc catccgcact | 600 |
| gccacagaga agccgctggg ggagctatgg aagtga | 636 |

```
<211> 211
<212> PRT
<213> Homo sapiens
Met Ala Ala Arg Arg Gly Ala Leu Ile Val Leu Glu Gly Val Asp Arg
1 5 10 15
Ala Gly Lys Ser Thr Gln Ser Arg Lys Leu Val Glu Ala Leu Ser Arg
                        20 25 30
Gly Pro Pro Pro Glu Leu Leu Arg Phe Pro Glu Arg Ser Thr Glu Ile
                35 40 45
Gly Lys Leu Leu Ser Ser Tyr Leu Gln Lys Lys Ser Asp Val Glu Asp
        50 55 60
His Ser Val His Leu Leu Phe Ser Ala Asn Arg Trp Glu Gln Val Pro
65 70 75 80
Leu Ile Lys Glu Lys Leu Ser Gln Gly Val Thr Leu Val Val Asp Arg
                                85 90 95
Tyr Ala Phe Ser Gly Val Ala Phe Thr Gly Ala Lys Glu Asn Phe Ser
                        100 105 110
Leu Asp Trp Cys Lys Gln Pro Asp Val Gly Leu Pro Lys Pro Asp Leu
                115 120 125
Val Leu Phe Leu Gln Leu Gln Leu Ala Asp Ala Ala Lys Arg Gly Ala
        130 135 140
Phe Gly His Glu Arg Tyr Glu Asn Gly Ala Phe Gln Glu Arg Ala Leu
145 150 155 160
Arg Cys Phe His Gln Leu Met Lys Asp Thr Thr Leu Asn Trp Lys Met
                                165 170 175
Val Asp Ala Ser Lys Arg Leu Glu Ala Val His Glu Glu Leu Arg Val
                        180 185 190
Leu Ser Glu Asp Ala Ile Arg Thr Ala Thr Glu Lys Pro Leu Gly Glu
                195 200 205
Leu Trp Lys
        210
<211> 639
<212> DNA
<213> Homo sapiens
```

| | |
|---|---|
| atggcggccc ggcgcggggc tctcatagtg ctggagggcg tggaccgcgc cgggaagagc | 60 |
| acgcagagcc gcaagctggt ggaagcgctg tgcgccgcgg gccaccgcgc cgaactgctc | 120 |
| cggttcccgg aaagatcaac tgaaatcggc aaacttctga gttcctactt gcaaaagaaa | 180 |
| agtgacgtgg aggatcactc ggtgcacctg cttttttctg caaatcgctg ggaacaagtg | 240 |
| ccgttaatta aggaaaagtt gagccagggc gtgaccctcg tcgtggacag atacgcattt | 300 |
| tctggtgtgg ccttcaccgg tgccaaggag aatttttccc tagattggtg taaacagcca | 360 |
| gacgtgggcc ttcccaaacc cgacctggtc ctgttcctcc agttacagct ggcggatgct | 420 |
| gccaagcggg gagcgtttgg ccatgagcgc tatgagaacg gggctttcca ggagcgggcg | 480 |
| ctccggtgtt tccaccagct catgaaagac acgactttga actggaagat ggtggatgct | 540 |
| tccaaaagca tcgaagctgt ccatgaggac atccgcgtgc tctctgagga cgccatccgc | 600 |
| actgccacag agaagccgct gggggagcta tggaaggac | 639 |

```
<211> 213
<212> PRT
<213> Homo sapiens
Met Ala Ala Arg Arg Gly Ala Leu Ile Val Leu Glu Gly Val Asp Arg
1 5 10 15
Ala Gly Lys Ser Thr Gln Ser Arg Lys Leu Val Glu Ala Leu Cys Ala
                        20 25 30
Ala Gly His Arg Ala Glu Leu Leu Arg Phe Pro Glu Arg Ser Thr Glu
                35 40 45
Ile Gly Lys Leu Leu Ser Ser Tyr Leu Gln Lys Lys Ser Asp Val Glu
        50 55 60
Asp His Ser Val His Leu Leu Phe Ser Ala Asn Arg Trp Glu Gln Val
65 70 75 80
Pro Leu Ile Lys Glu Lys Leu Ser Gln Gly Val Thr Leu Val Val Asp
                                85 90 95
Arg Tyr Ala Phe Ser Gly Val Ala Phe Thr Gly Ala Lys Glu Asn Phe
                        100 105 110
Ser Leu Asp Trp Cys Lys Gln Pro Asp Val Gly Leu Pro Lys Pro Asp
                115 120 125
Leu Val Leu Phe Leu Gln Leu Gln Leu Ala Asp Ala Ala Lys Arg Gly
        130 135 140
Ala Phe Gly His Glu Arg Tyr Glu Asn Gly Ala Phe Gln Glu Arg Ala
145 150 155 160
Leu Arg Cys Phe His Gln Leu Met Lys Asp Thr Thr Leu Asn Trp Lys
                                165 170 175
Met Val Asp Ala Ser Lys Ser Ile Glu Ala Val His Glu Asp Ile Arg
                        180 185 190
Val Leu Ser Glu Asp Ala Ile Arg Thr Ala Thr Glu Lys Pro Leu Gly
                195 200 205
Glu Leu Trp Lys Asp
        210
<211> 639
<212> DNA
<213> Mus musculus
```

| | |
|---|---|
| atggcgtcgc gtcggggagc gctcatcgtg ctggagggtg tggaccgtgc tggcaagacc | 60 |
| acgcagggcc tcaagctggt gaccgcgctg tgcgcctcgg gccacagagc ggagctgctg | 120 |
| cgtttccccg aaagatcaac ggaaatcggc aagcttctga attcctactt ggaaaagaaa | 180 |
| acggaactag aggatcactc cgtgcacctg ctcttctctg caaaccgctg ggaacaagta | 240 |
| ccattaatta aggcgaagtt gaaccagggt gtgacccttg ttttggacag atacgccttt | 300 |
| tctggggttg ccttcactgg tgccaaagag aatttttccc tggattggtg taaacaaccg | 360 |
| gacgtgggcc ttcccaaacc tgacctgatc ctgttccttc agttacaatt gctggacgct | 420 |
| gctgcacggg gagagtttgg ccttgagcga tatgagaccg ggactttcca aaagcaggtt | 480 |
| ctgttgtgtt tccagcagct catggaagag aaaaacctca actggaaggt ggttgatgct | 540 |
| tccaaaagca ttgaggaagt ccataaagaa atccgtgcac actctgagga cgccatccga | 600 |
| aacgctgcac agaggccact gggggagcta tggaaataa | 639 |

```
<211> 212
<212> PRT
<213> Mus musculus
Met Ala Ser Arg Arg Gly Ala Leu Ile Val Leu Glu Gly Val Asp Arg
1 5 10 15
Ala Gly Lys Thr Thr Gln Gly Leu Lys Leu Val Thr Ala Leu Cys Ala
                        20 25 30
Ser Gly His Arg Ala Glu Leu Leu Arg Phe Pro Glu Arg Ser Thr Glu
                35 40 45
Ile Gly Lys Leu Leu Asn Ser Tyr Leu Glu Lys Lys Thr Glu Leu Glu
        50 55 60
Asp His Ser Val His Leu Leu Phe Ser Ala Asn Arg Trp Glu Gln Val
65 70 75 80
Pro Leu Ile Lys Ala Lys Leu Asn Gln Gly Val Thr Leu Val Leu Asp
                                85 90 95
Arg Tyr Ala Phe Ser Gly Val Ala Phe Thr Gly Ala Lys Glu Asn Phe
                        100 105 110
Ser Leu Asp Trp Cys Lys Gln Pro Asp Val Gly Leu Pro Lys Pro Asp
                115 120 125
Leu Ile Leu Phe Leu Gln Leu Gln Leu Leu Asp Ala Ala Ala Arg Gly
        130 135 140
Glu Phe Gly Leu Glu Arg Tyr Glu Thr Gly Thr Phe Gln Lys Gln Val
145 150 155 160
Leu Leu Cys Phe Gln Gln Leu Met Glu Glu Lys Asn Leu Asn Trp Lys
                                165 170 175
Val Val Asp Ala Ser Lys Ser Ile Glu Glu Val His Lys Glu Ile Arg
                        180 185 190
Ala His Ser Glu Asp Ala Ile Arg Asn Ala Ala Gln Arg Pro Leu Gly
                195 200 205
Glu Leu Trp Lys
        210
<211> 212
<212> PRT
<213> Homo sapiens
Met Ala Ala Arg Arg Gly Ala Leu Ile Val Leu Glu Gly Val Asp Arg
1 5 10 15
Ala Gly Lys Ser Thr Gln Ser Arg Lys Leu Val Glu Ala Leu Cys Ala
                        20 25 30
Ala Gly His Arg Ala Glu Leu Leu Arg Phe Pro Glu Arg Ser Thr Glu
                35 40 45
Ile Gly Lys Leu Leu Ser Ser Tyr Leu Gln Lys Lys Ser Asp Val Glu
        50 55 60
Asp His Ser Val His Leu Leu Phe Ser Ala Asn Arg Trp Glu Gln Val
65 70 75 80
Pro Leu Ile Lys Glu Lys Leu Ser Gln Gly Val Thr Leu Val Val Asp
                                85 90 95
Arg Tyr Ala Phe Ser Gly Val Ala Tyr Thr Gly Ala Lys Glu Asn Phe
                        100 105 110
Ser Leu Asp Trp Cys Lys Gln Pro Asp Val Gly Leu Pro Lys Pro Asp
                115 120 125
Leu Val Leu Phe Leu Gln Leu Gln Leu Ala Asp Ala Ala Lys Arg Gly
        130 135 140
Ala Phe Gly His Glu Arg Tyr Glu Asn Gly Ala Phe Gln Glu Arg Ala
145 150 155 160
Leu Arg Cys Phe His Gln Leu Met Lys Asp Thr Thr Leu Asn Trp Lys
                                165 170 175
Met Val Asp Ala Ser Lys Ser Ile Glu Ala Val His Glu Asp Ile Arg
                        180 185 190
Val Leu Ser Glu Asp Ala Ile Arg Thr Ala Thr Glu Lys Pro Leu Gly
                195 200 205
Glu Leu Trp Lys
        210
<211> 214
<212> PRT
<213> Homo sapiens
Met Ala Ala Arg Arg Gly Ala Leu Ile Val Leu Glu Gly Val Asp Gly
1 5 10 15
Ala Gly Lys Ser Thr Gln Ser Arg Lys Leu Val Glu Ala Leu Cys Ala
                        20 25 30
Ala Gly His Arg Ala Glu Leu Leu Arg Phe Pro Glu Arg Ser Thr Glu
                35 40 45
Ile Gly Lys Leu Leu Ser Ser Tyr Leu Gln Lys Lys Ser Asp Val Glu
        50 55 60
Asp His Ser Val His Leu Leu Phe Ser Ala Asn Arg Trp Glu Gln Val
65 70 75 80
Pro Leu Ile Lys Glu Lys Leu Ser Gln Gly Val Thr Leu Val Val Asp
                                85 90 95
Arg Tyr Ala Phe Ser Gly Val Ala Phe Thr Gly Ala Lys Glu Asn Phe
                        100 105 110
Ser Leu Asp Trp Cys Lys Gln Pro Asp Val Gly Leu Pro Lys Pro Asp
                115 120 125
Leu Val Leu Phe Leu Gln Leu Thr Pro Glu Val Gly Leu Lys Arg Ala
        130 135 140
Arg Ala Arg Gly Gln Leu Asp Arg Tyr Glu Asn Gly Ala Phe Gln Glu
145 150 155 160
Arg Ala Leu Arg Cys Phe His Gln Leu Met Lys Asp Thr Thr Leu Asn
                                165 170 175
Trp Lys Met Val Asp Ala Ser Lys Ser Ile Glu Ala Val His Glu Asp
                        180 185 190
Ile Arg Val Leu Ser Glu Asp Ala Ile Ala Thr Ala Thr Glu Lys Pro
                195 200 205
Leu Gly Glu Leu Trp Lys
        210
pORF-hIL-12 sequence (5048 bp).
hIL-12 open reading frame in bold.
Elastin linker is underlined.
GGATCTGCGATCGCTCCGGTGCCCGTCAGTGGGCAGAGCGCACATCGCCCACAGTCCCCGAGAAGTTGGGGGGAG
GGGTCGGCAATTGAACCGGTGCCTAGAGAAGGTGGCGCGGGGTAAACTGGGAAAGTGATGTCGTGTACTGGCTCC
GCCTTTTTCCCGAGGGTGGGGGAGAACCGTATATAAGTGCAGTAGTCGCCGTGAACGTTCTTTTTCGCAACGGGTT
TGCCGCCAGAACACAGCTGAAGCTTCGAGGGGCTCGCATCTCTCCTTCACGCGCCCGCCGCCCTACCTGAGGCCGC
CATCCACGCCGGTTGAGTCGCGTTCTGCCGCCTCCCGCCTGTGGTGCCTCCTGAACTGCGTCCGCCGTCTAGGTAA
GTTTAAAGCTCAGGTCGAGACCGGGCCTTTGTCCGGCGCTCCCTTGGAGCCTACCTAGACTCAGCCGGCTCTCCAC
GCTTTGCCTGACCCTGCTTGCTCAACTCTACGTCTTTGTTTCGTTTTCTGTTCTGCGCCGTTACAGATCCAAGCTGTG
ACCGGCGCCTACgtaagtgatatctactagatttatcaaaaagagtgttgacttgtgagcgctcacaattgatactta
gattcatcgagagggacacgtcgactactaaccttcttact
ttectacagCTGAGATCACCGGCGAAGGAGGGCCACCATGGGTCACCAGCAGTTGGTCATCTCTTGGTTTTCCCTGG
TTTTTCTGGCATCTCCCCTCGTGGCCATATGGGAACTGAAGAAAGATGTTTATGTCGTAGAATTGGATTGGT
ATCCGGATGCCCCTGGAGAAATGGTGGTCCTCACCTGTGACACCCCTGAAGAAGATGGTATCACCTGGACC
TTGGACCAGAGCAGTGAGGTCTTAGGCTCTGGCAAAACCCTGACCATCCAAGTCAAAGAGTTTGGAGATGC
TGGCCAGTACACCTGTCACAAAGGAGGCGAGGTTCTAAGCCATTCGCTCCTGCTGCTTCACAAAAAGGAAG
ATGGAATTTGGTCCACTGATATTTTAAAGGACCAGAAAGAACCCAAAAATAAGACCTTTCTAAGATGCGAGG
CCAAGAATTATTCTGGACGTTTCACCTGCTGGTGGCTGACGACAATCAGTACTGATTTGACATTCAGTGTCA
AAAGCAGCAGAGGCTCTTCTGACCCCCAAGGGGTGACGTGCGGAGCTGCTACACTCTCTGCAGAGAGAGTC
AGAGGGGACAACAAGGAGTATGAGTACTCAGTGGAGTGCCAGGAGGACAGTGCCTGCCCAGCTGCTGAGG
AGAGTCTGCCCATTGAGGTCATGGTGGATGCCGTTCACAAGCTCAAGTATGAAAACTACACCAGCAGCTTC
TTCATCAGGGACATCATCAAACCTGACCCACCCAAGAACTTGCAGCTGAAGCCATTAAAGAATTCTCGGCA
GGTGGAGGTCAGCTGGGAGTACCCTGACACCTGGAGTACTCCACATTCCTACTTCTCCCTGACATTCTGCG
TTCAGGTCCAGGGCAAGAGCAAGAGAGAAAAGAAAGATAGAGTCTTCACGGACAAGACCTCAGCCACGGTC
ATCTGCCGCAAAAATGCCAGCATTAGCGTGCGGGCCCAGGACCGCTACTATAGCTCATCTTGGAGCGAATG
GGCATCTGTGCCCTGCAGTGTTCCTGGAGTAGGGGTACCTGGGGTGGGCGCCAGAAACCTCCCCGTGGCC
ACTCCAGACCCAGGAATGTTCCCATGCCTTCACCACTCCCAAAACCTGCTGAGGGCCGTCAGCAACATGCT
CCAGAAGGCCAGACAAACTCTAGAATTTTACCCTTGCACTTCTGAAGAGATTGATCATGAAGATATCACAAA
AGATAAAACCAGCACAGTGGAGGCCTGTTTACCATTGGAATTAACCAAGAATGAGAGTTGCCTAAATTCCA
GAGAGACCTCTTTCATAACTAATGGGAGTTGCCTGGCCTCCAGAAAGACCTCTTTTATGATGGCCCTGTGCC
TTAGTAGTATTTATGAAGACTCGAAGATGTACCAGGTGGAGTTCAAGACCATGAATGCAAAGCTTCTGATG
GATCCTAAGAGGCAGATCTTTCTAGATCAAAACATGCTGGCAGTTATTGATGAGCTGATGCAGGCCCTGAA
TTTCAACAGTGAGACTGTGCCACAAAAATCCTCCCTTGAAGAACCGGATTTTTATAAAACTAAAATCAAGCT
CTGCATACTTCTTCATGCTTTCAGAATTCGGGCAGTGACTATTGATAGAGTGATGAGCTATCTGAATGCTTC
CTAAAAAGCGAGGTCCCTCCAAACCGTTGTCATTTTTATAAAACTTTGAAATGAGGAAACTTTGATAGGATGTGGA
TTAAGAACTAGGGAGGGGGAAAGAAGGATGGGACTATTACATCCACATGATACCTCTGATCAAGTATTTTTGACA
TTTACTGTGGATAAATTGTTTTTAAGTTTTCATGAATGAATTGCTAAGAAGGGGGGAATTCTTTTGCTTTTTACCCT
CGACTAGCTCGACATGATAAGATACATTGATGAGTTTGGACAAACCACAACTAGAATGCAGTGAAAAAAATGCTT
TATTTGTGAAATTTGTGATGCTATTGCTTTATTTGTGAAATTTGTGATGCTATTGCTTTATTTGTAACCATTATAAGC
TGCAATAAACAAGTTAACAACAACAATTGCATTCATTTTATGTTTCAGGTTCAGGGGGAGGTGTGGGAGGTTTTTT
AAAGCAAGTAAAACCTCTACAAATGTGGTAGATCATTTAAATGTTAATTAAGAACATGTGAGCAAAAGGCCAGCA
AAAGGCCAGGAACCGTAAAAAGGCCGCGTTGCTGGCGTTTTTCCATAGGCTCCGCCCCCCTGACGAGCATCACAA
AAATCGACGCTCAAGTCAGAGGTGGCGAAACCCGACAGGACTATAAAGATACCAGGCGTTTCCCCCTGGAAGCTC
CCTCGTGCGCTCTCCTGTTCCGACCCTGCCGCTTACCGGATACCTGTCCGCCTTTCTCCCTTCGGGAAGCGTGGCGC
TTTCTCAATGCTCACGCTGTAGGTATCTCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTCCAACCCGGTAAGACACGACTTATCGC
CACTGGCAGCAGCCACTGGTAACAGGATTAGCAGAGCGAGGTATGTAGGCGGTGCTACAGAGTTCTTGAAGTGGT
GGCCTAACTACGGCTACACTAGAAGAACAGTATTTGGTATCTGCGCTCTGCTGAAGCCAGTTACCTTCGGAAAAAG
AGTTGGTAGCTCTTGATCCGGCAAACAAACCACCGCTGGTAGCGGTGGTTTTTTTGTTTGCAAGCAGCAGATTACG
CGCAGAAAAAAAGGATCTCAAGAAGATCCTTTGATCTTTTCTACGGGGTCTGACGCTCAGTGGAACGAAAACTCA
CGTTAAGGGATTTTGGTCATGAGATTATCAAAAAGGATCTTCACCTAGATCCTTTTAAATTAAAAATGAAGTTTTA
AATCAATCTAAAGTATATATGAGTAAACTTGGTCTGACAGTTACCAATGCTTAATCAGTGAGGCACCTATCTCAGC
GATCTGTCTATTTCGTTCATCCATAGTTGCCTGACTCCCCGTCGTGTAGATAACTACGATACGGGAGGGCTTACCAT
CTGGCCCCAGTGCTGCAATGATACCGCGAGACCCACGCTCACCGGCTCCAGATTTATCAGCAATAAACCAGCCAG
CCGGAAGGGCCGAGCGCAGAAGTGGTCCTGCAACTTTATCCGCCTCCATCCAGTCTATTAATTGTTGCCGGGAAGC
TAGAGTAAGTAGTTCGCCAGTTAATAGTTTGCGCAACGTTGTTGCCATTGCTACAGGCATCGTGGTGTCACGCTCG
TCGTTTGGTATGGCTTCATTCAGCTCCGGTTCCCAACGATCAAGGCGAGTTACATGATCCCCCATGTTGTGCAAAA
AAGCGGTTAGCTCCTTCGGTCCTCCGATCGTTGTCAGAAGTAAGTTGGCCGCAGTGTTATCACTCATGGTTATGGC
AGCACTGCATAATTCTCTTACTGTCATGCCATCCGTAAGATGCTTTTCTGTGACTGGTGAGTACTCAACCAAGTCAT
TCTGAGAATAGTGTATGCGGCGACCGAGTTGCTCTTGCCCGGCGTCAATACGGGATAATACCGCGCCACATAGCA
GAACTTTAAAAGTGCTCATCATTGGAAAACGTTCTTCGGGGCGAAAACTCTCAAGGATCTTACCGCTGTTGAGATC
CAGTTCGATGTAACCCACTCGTGCACCCAACTGATCTTCAGCATCTTTTACTTTCACCAGCGTTTCTGGGTGAGCAA
AAACAGGAAGGCAAAATGCCGCAAAAAAGGGAATAAGGGCGACACGGAAATGTTGAATACTCATACTCTTCCTTT
TTCAATATTATTGAAGCATTTATCAGGGTTATTGTCTCATGAGCGGATACATATTTGAATGTATTTAGAAAAATAA
ACAAATAGGGGTTCCGCGCACATTTCCCCGAAAAGTGCCACCTGACGTCTAAGAAACCATTATTATCATGACATTA
ACCTATAAAAATAGGCGTATCACGAGGCCCTTTCGTCTCGCGCGTTTCGGTGATGACGGTGAAAACCTCTGACACA
TGCAGCTCCCGGAGACGGTCACAGCTTGTCTGTAAGCGGATGCCGGGAGCAGACAAGCCCGTCAGGGCGCGTCAG
CGGGTGTTGGCGGGTGTCGGGGCTGGCTTAACTATGCGGCATCAGAGCAGATTGTACTGAGAGTGCACCATATGG
ATCTCGAGCGGCCGCAATAAAATATCTTTATTTTCATTACATCTGTGTGTTGGTTTTTTGTGTGAATCGTAACTAAC
ATACGCTCTCCATCAAAACAAAACGAAACAAAACAAACTAGCAAAATAGGCTGTCCCCAGTGCAAGTGCAGGTGC
CAGAACATTTCTCTATCGAA
pORF-mIL-12 (p35p40) sequence (4846 bp).
mIL-12 open reading frame in bold.
Elastin linker sequence is undelined.
GGATCTGCGATCGCTCCGGTGCCCGTCAGTGGGCAGAGCGCACATCGCCCACAGTCCCCGAGAAGTTGGGGGGAG
GGGTCGGCAATTGAACCGGTGCCTAGAGAAGGTGGCGCGGGGTAAACTGGGAAAGTGATGTCGTGTACTGGCTCC
GCCTTTTTCCCGAGGGTGGGGGAGAACCGTATATAAGTGCAGTAGTCGCCGTGAACGTTCTTTTTCGCAACGGGTT
TGCCGCCAGAACACAGCTGAAGCTTCGAGGGGCTCGCATCTCTCCTTCACGCGCCCGCCGCCCTACCTGAGGCCGC
CATCCACGCCGGTTGAGTCGCGTTCTGCCGCCTCCCGCCTGTGGTGCCTCCTGAACTGCGTCCGCCGTCTAGGTAA
GTTTAAAGCTCAGGTCGAGACCGGGCCTTTGTCCGGCGCTCCCTTGGAGCCTACCTAGACTCAGCCGGCTCTCCAC
GCTTTGCCTGACCCTGCTTGCTCAACTCTACGTCTTTGTTTCGTTTTCTGTTCTGCGCCGTTACAGATCCAAGCTGTG
ACCGGCGCCTACgtaagtgatatctactagatttatcaaaaagagtgttgacttgtgagcgctcacaattgatactta
gattcatcgagagggacacgtcgactactaaccttcttact
ttectacagCTGAGATCACCGGCGAAGGAGGGCCACCATGGGTCAATCACGCTACCTCCTCTTTTTGGCCACCCTTG
CCCTCCTAAACCACCTCAGTTTGGCCAGGGTCATTCCAGTCTCTGGACCTGCCAGGTGTCTTAGCCAGTCCC
GAAACCTGCTGAAGACCACAGATGACATGGTGAAGACGGCCAGAGAAAAGCTGAAACATTATTCCTGCACT
GCTGAAGACATCGATCATGAAGACATCACACGGGACCAAACCAGCACATTGAAGACCTGTTTACCACTGGA
ACTACACAAGAACGAGAGTTGCCTGGCTACTAGAGAGACTTCTTCCACAACAAGAGGGAGCTGCCTGCCCC
CACAGAAGACGTCTTTGATGATGACCCTGTGCCTTGGTAGCATCTATGAGGACTTGAAGATGTACCAGACA
GAGTTCCAGGCCATCAACGCAGCACTTCAGAATCACAACCATCAGCAGATCATTCTAGACAAGGGCATGCT
GGTGGCCATCGATGAGCTGATGCAGTCTCTGAATCATAATGGCGAGACTCTGCGCCAGAAACCTCCTGTGG
GAGAAGCAGACCCTTACAGAGTGAAAATGAAGCTCTGCATCCTGCTTCACGCCTTCAGCACCCGCGTCGTG
ACCATCAACAGGGTGATGGGCTATCTGAGCTCCGCCGTTCCTGGAGTAGGGGTACCTGGAGTGGGCGGATC
TATGTGGGAGCTGGAGAAAGACGTTTATGTTGTAGAGGTGGACTGGACTCCCGATGCCCCTGGAGAAACAG
TGAACCTCACCTGTGACACGCCTGAAGAAGATGACATCACCTGGACCTCAGACCAGAGACATGGAGTCATA
GGCTCTGGAAAGACCCTGACCATCACTGTCAAAGAGTTTCTAGATGCTGGCCAGTACACCTGCCACAAAGG
AGGCGAGACTCTGAGCCACTCACATCTGCTGCTCCACAAGAAGGAAAATGGAATTTGGTCCACTGAAATTT
TAAAAAATTTCAAAAACAAGACTTTCCTGAAGTGTGAAGCACCAAATTACTCCGGACGGTTCACGTGCTCAT
GGCTGGTGCAAAGAAACATGGACTTGAAGTTCAACATCAAGAGCAGTAGCAGTCCCCCCGACTCTCGGGCA
GTGACATGTGGAATGGCGTCTCTGTCTGCAGAGAAGGTCACACTGGACCAAAGGGACTATGAGAAGTATTC
AGTGTCCTGCCAGGAGGATGTCACCTGCCCAACTGCCGAGGAGACCCTGCCCATTGAACTGGCGTTGGAAG
CACGGCAGCAGAATAAATATGAGAACTACAGCACCAGCTTCTTCATCAGGGACATCATCAAACCAGACCCG
CCCAAGAACTTGCAGATGAAGCCTTTGAAGAACTCACAGGTGGAGGTCAGCTGGGAGTACCCTGACTCCTG
GAGCACTCCCCATTCCTACTTCTCCCTCAAGTTCTTTGTTCGAATCCAGCGCAAGAAAGAAAAGATGAAGGA
GACAGAGGAGGGGTGTAACCAGAAAGGTGCGTTCCTCGTAGAGAAGACATCTACCGAAGTCCAATGCAAAG
GCGGGAATGTCTGCGTGCAAGCTCAGGATCGCTATTACAATTCCTCATGCAGCAAGTGGGCATGTGTTCCC
TGCAGGGTCCGATCCTAGGATGCAACGGATGCTAGCTCGACATGATAAGATACATTGATGAGTTTGGACAAACC
ACAACTAGAATGCAGTGAAAAAAATGCTTTATTTGTGAAATTTGTGATGCTATTGCTTTATTTGTGAAATTTGTGAT
GCTATTGCTTTATTTGTAACCATTATAAGCTGCAATAAACAAGTTAACAACAACAATTGCATTCATTTTATGTTTCA
GGTTCAGGGGGAGGTGTGGGAGGTTTTTTAAAGCAAGTAAAACCTCTACAAATGTGGTAGATCATTTAAATGTTA
ATTAAGAACATGTGAGCAAAAGGCCAGCAAAAGGCCAGGAACCGTAAAAAGGCCGCGTTGCTGGCGTTTTTCCAT
AGGCTCCGCCCCCCTGACGAGCATCACAAAAATCGACGCTCAAGTCAGAGGTGGCGAAACCCGACAGGACTATAA
AGATACCAGGCGTTTCCCCCTGGAAGCTCCCTCGTGCGCTCTCCTGTTCCGACCCTGCCGCTTACCGGATACCTGTC
CGCCTTTCTCCCTTCGGGAAGCGTGGCGCTTTCTCAATGCTCACGCTGTAGGTATCTCAGTTCGGTGTAGGTCGTTC
GCTCCAAGCTGGGCTGTGTGCACGAACCCCCCGTTCAGCCCGACCGCTGCGCCTTATCCGGTAACTATCGTCTTGA
GTCCAACCCGGTAAGACACGACTTATCGCCACTGGCAGCAGCCACTGGTAACAGGATTAGCAGAGCGAGGTATGT
AGGCGGTGCTACAGAGTTCTTGAAGTGGTGGCCTAACTACGGCTACACTAGAAGAACAGTATTTGGTATCTGCGCT
CTGCTGAAGCCAGTTACCTTCGGAAAAAGAGTTGGTAGCTCTTGATCCGGCAAACAAACCACCGCTGGTAGCGGT
GGTTTTTTTGTTTGCAAGCAGCAGATTACGCGCAGAAAAAAAGGATCTCAAGAAGATCCTTTGATCTTTTCTACGG
GGTCTGACGCTCAGTGGAACGAAAACTCACGTTAAGGGATTTTGGTCATGAGATTATCAAAAAGGATCTTCACCTA
GATCCTTTTAAATTAAAAATGAAGTTTTAAATCAATCTAAAGTATATATGAGTAAACTTGGTCTGACAGTTACCAA
TGCTTAATCAGTGAGGCACCTATCTCAGCGATCTGTCTATTTCGTTCATCCATAGTTGCCTGACTCCCCGTCGTGTA
GATAACTACGATACGGGAGGGCTTACCATCTGGCCCCAGTGCTGCAATGATACCGCGAGACCCACGCTCACCGGC
TCCAGATTTATCAGCAATAAACCAGCCAGCCGGAAGGGCCGAGCGCAGAAGTGGTCCTGCAACTTTATCCGCCTC
CATCCAGTCTATTAATTGTTGCCGGGAAGCTAGAGTAAGTAGTTCGCCAGTTAATAGTTTGCGCAACGTTGTTGCC
ATTGCTACAGGCATCGTGGTGTCACGCTCGTCGTTTGGTATGGCTTCATTCAGCTCCGGTTCCCAACGATCAAGGC
GAGTTACATGATCCCCCATGTTGTGCAAAAAAGCGGTTAGCTCCTTCGGTCCTCCGATCGTTGTCAGAAGTAAGTT
GGCCGCAGTGTTATCACTCATGGTTATGGCAGCACTGCATAATTCTCTTACTGTCATGCCATCCGTAAGATGCTTTT
CTGTGACTGGTGAGTACTCAACCAAGTCATTCTGAGAATAGTGTATGCGGCGACCGAGTTGCTCTTGCCCGGCGTC
AATACGGGATAATACCGCGCCACATAGCAGAACTTTAAAAGTGCTCATCATTGGAAAACGTTCTTCGGGGCGAAA
ACTCTCAAGGATCTTACCGCTGTTGAGATCCAGTTCGATGTAACCCACTCGTGCACCCAACTGATCTTCAGCATCTT
TTACTTTCACCAGCGTTTCTGGGTGAGCAAAAACAGGAAGGCAAAATGCCGCAAAAAAGGGAATAAGGGCGACA
CGGAAATGTTGAATACTCATACTCTTCCTTTTTCAATATTATTGAAGCATTTATCAGGGTTATTGTCTCATGAGCGG
ATACATATTTGAATGTATTTAGAAAAATAAACAAATAGGGGTTCCGCGCACATTTCCCCGAAAAGTGCCACCTGAC
GTCTAAGAAACCATTATTATCATGACATTAACCTATAAAAATAGGCGTATCACGAGGCCCTTTCGTCTCGCGCGTT
TCGGTGATGACGGTGAAAACCTCTGACACATGCAGCTCCCGGAGACGGTCACAGCTTGTCTGTAAGCGGATGCCG
GGAGCAGACAAGCCCGTCAGGGCGCGTCAGCGGGTGTTGGCGGGTGTCGGGGCTGGCTTAACTATGCGGCATCAG
AGCAGATTGTACTGAGAGTGCACCATATGGATCTCGAGCGGCCGCAATAAAATATCTTTATTTTCATTACATCTGT
GTGTTGGTTTTTTGTGTGAATCGTAACTAACATACGCTCTCCATCAAAACAAAACGAAACAAAACAAACTAGCAA
AATAGGCTGTCCCCAGTGCAAGTGCAGGTGCCAGAACATTTCTCTATCGAA
```

1. A composition, vector construct or virus comprising:
a stably integrating delivery vector;
a tumor associated antigen cassette;
a lysomal targeting cassette;
wherein the lysosomal targeting cassette is operatively linked to the tumor associated antigen cassette.

2. The composition, vector construct or virus of claim 1, wherein the delivery vector comprises a retroviral vector, optionally a lentiviral vector, wherein the lentiviral vector comprises one or more of a: 5′-Long terminal repeat (LTR), HIV signal sequence, HIV Psi signal 5′-splice site (SD), delta-GAG element, Rev Responsive Element (RRE), 3′-splice site (SA), Elongation factor (EF) 1-alpha promoter and 3′-Self inactivating LTR (SIN-LTR).

3. (canceled)

4. The composition, vector construct or virus of claim 2, wherein the lentiviral vector comprises a central polypurine tract and/or a woodchuck hepatitis virus post-transcriptional regulatory element; optionally wherein the cPPT comprises SEQ ID NO:2 and/or the WPRE comprises SEQ ID NO:3; or, optionally wherein the cPPT comprises at least 70% sequence identity to SEQ ID NO:2 and/or a the WPRE comprises at least 70% sequence identity to SEQ ID NO:3, wherein the lentiviral vector comprises the nucleotides corresponding to a pHR′ vector backbone.

5. (canceled)

6. (canceled)

7. The composition, vector construct or virus of claim 1, wherein the tumor associated antigen cassette comprises all or part of a carcinoembryonic antigen polynucleotide or wherein the tumor associated antigen cassette comprises all or part of a HER-2/neu polynucleotide.

8-10. (canceled)

11. The composition, vector construct or virus of claim 1, wherein the lysosomal targeting cassette comprises a LAMP1 lysosomal targeting polynucleotide, wherein the LAMP1 lysosomal targeting polynucleotide is selected from the group consisting of SEQ ID NO:1 or a polynucleotide having at least 70% sequence identity to SEQ ID NO:1 which maintains lysosomal targeting activity.

12. (canceled)

13. The composition, vector construct or virus of claim 1, further comprising an activator polynucleotide encoding a polypeptide that converts a prodrug to a drug, optionally a modified tmpk polynucleotide and/or a tmpk polynucleotide with at least 80% sequence identity to a modified tmpk polynucleotide described herein, and optionally further comprising a detection cassette.

14. (canceled)

15. The composition, vector construct or virus of claim 13, wherein the detection cassette is selected from CD19, truncated CD19, CD20, human CD24, murine HSA, human CD25 (huCD25), a truncated form of low affinity nerve growth factor receptor (LNGFR), truncated CD34 or erythropoietin receptor (EpoR) polynucleotides and/or a polynucleotide comprising at least 70% sequence identity to a CD19, truncated CD19, CD20, human CD24, murine HSA, CD25, a truncated form of low affinity nerve growth factor receptor (LNGFR), truncated CD34 or erythropoietin receptor (EpoR)polynucleotide.

16. The composition, vector construct or virus of claims 1-1-5, further comprising an immune modulatory cassette, wherein the immune modulatory cassette comprises a polynucleotide selected from the group comprising IL-12 p35, IL-12 p40, IL-12 fusion, IL-15, RANKL, CD40L, IFNγ and TNFα polynucleotides and combinations thereof, or wherein the immune modulatory cassette encodes a protein that modulates dendritic cells, encodes a protein that modulates T cells, optionally CD4+ T cells.

17-20. (canceled)

21. The composition, vector construct or virus of claim 20, wherein the IL-12 polynucleotide is a mammalian IL-12 polynucleotide, or wherein the IL-12 polynucleotide comprises at least 70% sequence identity to sequences having SEQ ID NO:16-19.

22-25. (canceled)

26. A cell transduced with the composition claim 1 vector construct, or the virus of claim 1, wherein the cell is optionally an antigen presenting cell, a stem cell, immune cell, hematopoietic cell, dendritic cell, or an immature dendritic cell and/or a population of cells comprising the transduced cell.

27. A method of expressing a tumor associated antigen in a mammalian cell comprising contacting the mammalian cell with the composition vector construct, or virus of claim 1, optionally wherein the mammalian cell is selected from a stem cell, an immune cell, a hematopoietic cell, an antigen presenting cell, a cancer cell and a dendritic cell.

28-32. (canceled)

33. The method of claim 27, further comprising a step of treating the transduced cell with a cell maturing agent, wherein the cell maturing agent is TNFα.

34. (canceled)

35. The method of claim 27, further comprising a step of isolating the transduced cells, and/or further comprising a step wherein the isolated mammalian cells are transplanted in a mammal.

36. (canceled)

37. A method of treating a subject in need thereof, optionally a subject with cancer or an increased risk of developing cancer, comprising administering to the subject in need thereof the composition, vector construct, or virus of claim 1.

38. A method of treating a subject in need thereof comprising administering to the subject in need thereof the tranduced cell or population of claim 26.

39. (canceled)

40. A method of reducing cancer burden in a subject having a CEA or HER-2/neu positive cancer comprising administering to the subject the composition of, vector construct, or virus of claim 4, or a transduced cell or population of cells comprising the composition, vector construct or virus.

41. (canceled)

42. (canceled)

43. The method of claim 37, wherein the cancer is colon cancer, rectal cancer, stomach cancer, pancreatic cancer, non-small cell lung cancer, metastatic pancreatic cancer, ovarian cancer or breast cancer.

44. The method of claim 38, wherein the transduced cell is a dendritic cell, an immature dendritic cell, or an autologous dendritic cell.

45. (canceled)

46. (canceled)

47. A method of inducing or enhancing an immune response in a subject in need thereof comprising administering to the subject in need thereof the composition, vector construct, the virus of claim 1, or a transduced cell or population of cells comprising the composition, vector construct or virus.

48. (canceled)

49. (canceled)

50. The method of claim 38 wherein the transduced cell or population is growth arrested or irradiated prior to administering to the subject.

51-68. (canceled)

69. The method of claim 38, wherein the number of cells administered ranges from 10⁵ cells to 10⁹ cells, optionally about 10⁵ cells, about 10⁶ cells, about 10⁷, cells, about 10⁸ cells, or about 10⁹ cells.

70. (canceled)


SEQUENCE LISTING (23 sequences)

SEQ ID NO: 1 - protein, 31 aa, Artificial Sequence
  Synthetic construct - Lysosomal Targeting Sequence
MQLRNPELHLGCALALRFLALVSWDIPGARA

SEQ ID NO: 2 - DNA, 118 bp, Artificial Sequence
  Synthetic construct -  CPPT
ttttaaaagaaaaggggggattggggggtacagtgcaggggaaagaatagtagacataatagcaacagacatacaaactaaagaattacaaaaacaaattacaaaaattcaaaatttt

SEQ ID NO: 3 - DNA, 592 bp, Artificial Sequence
  Synthetic construct - Woodchuck Hepatitus Virus WPRE
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 4 - DNA, 639 bp, Homo sapiens
  misc_feature: tmpk wt
atggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagattggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatccgcactgccacagagaagccgctgggggagctatggaagtga

SEQ ID NO: 5 - protein, 212 aa, Homo sapiens
  MISC_FEATURE: tmpk wt
MAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLQLADAAKRGAFGHERYENGAFQERALRCFHQLMKDTTLNWKMVDASKSIEAVHEDIRVLSEDAIRTATEKPLGELWK

SEQ ID NO: 6 - DNA, 639 bp, Homo sapiens; identical to SEQ ID NO: 4
  misc_feature: tmpk wt

SEQ ID NO: 7 - protein, 212 aa, Homo sapiens; identical to SEQ ID NO: 5
  MISC_FEATURE: tmpk wt

SEQ ID NO: 8 - DNA, 636 bp, Homo sapiens
  misc_feature: tmpk wt
atggcggcccggcgcggggctctcatagtgctggagggcgtggaccgcgccgggaagagcacgcagagccgcaagctggtggaagcgctgtcgcgcgggccaccgcccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagactcgaagctgtccatgaggaactccgcgtgctctctgaggacgccatccgcactgccacagagaagccgctgggggagctatggaagtga

SEQ ID NO: 9 - protein, 211 aa, Homo sapiens
  MISC_FEATURE: tmpk wt
MAARRGALIVLEGVDRAGKSTQSRKLVEALSRGPPPELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLQLADAAKRGAFGHERYENGAFQERALRCFHQLMKDTTLNWKMVDASKRLEAVHEELRVLSEDAIRTATEKPLGELWK

SEQ ID NO: 10 - DNA, 639 bp, Homo sapiens
  misc_feature: tmpk wt (no stop codon)
atggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagattggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatccgcactgccacagagaagccgctgggggagctatggaaggac

SEQ ID NO: 11 - protein, 213 aa, Homo sapiens
  MISC_FEATURE: tmpk wt putative sequence
MAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLQLADAAKRGAFGHERYENGAFQERALRCFHQLMKDTTLNWKMVDASKSIEAVHEDIRVLSEDAIRTATEKPLGELWKD

SEQ ID NO: 12 - DNA, 639 bp, Mus musculus
  misc_feature: tmpk wt
atggcgtcgcgtcggggagcgctcatcgtgctggagggtgtggaccgtgctggcaagaccacgcagggcctcaagctggtgaccgcgctgtgcgcctcgggccacagagcggagctgctgcgtttccccgaaagatcaacggaaatcggcaagcttctgaattcctacttggaaaagaaaacggaactagaggatcactccgtgcacctgctcttctctgcaaaccgctgggaacaagtaccattaattaaggcgaagttgaaccagggtgtgacccttgttttggacagatacgccttttctggggttgccttcactggtgccaaagagaatttttccctggattggtgtaaacaaccggacgtgggccttcccaaacctgacctgatcctgttccttcagttacaattgctggacgctgctgcacggggagagtttggccttgagcgatatgagaccgggactttccaaaagcaggttctgttgtgtttccagcagctcatggaagagaaaaacctcaactggaaggtggttgatgcttccaaaagcattgaggaagtccataaagaaatccgtgcacactctgaggacgccatccgaaacgctgcacagaggccactgggggagctatggaaataa

SEQ ID NO: 13 - protein, 212 aa, Mus musculus
  MISC_FEATURE: tmpk wt
MASRRGALIVLEGVDRAGKTTQGLKLVTALCASGHRAELLRFPERSTEIGKLLNSYLEKKTELEDHSVHLLFSANRWEQVPLIKAKLNQGVTLVLDRYAFSGVAFTGAKENFSLDWCKQPDVGLPKPDLILFLQLQLLDAAARGEFGLERYETGTFQKQVLLCFQQLMEEKNLNWKVVDASKSIEEVHKEIRAHSEDAIRNAAQRPLGELWK

SEQ ID NO: 14 - protein, 212 aa, Homo sapiens
  MISC_FEATURE: tmpk F105Y mutant
MAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAYTGAKENFSLDWCKQPDVGLPKPDLVLFLQLQLADAAKRGAFGHERYENGAFQERALRCFHQLMKDTTLNWKMVDASKSIEAVHEDIRVLSEDAIRTATEKPLGELWK

SEQ ID NO: 15 - protein, 214 aa, Homo sapiens
  MISC_FEATURE: tmpk R16GLL mutant
MAARRGALIVLEGVDGAGKSTQSRKLVEALCAAGHRAELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLTPEVGLKRARARGELDRYENGAFQERALRCFHQLMKDTTLNWKMVDASKSIEAVHEDIRVLSEDAIATATEKPLGELWK

SEQ ID NO: 16 - DNA, 5048 bp, Artificial Sequence
  Synthetic construct - pORF hIL-12 elasti (p40p35)
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagactcgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtccctccaaaccgttgtcatttttataaaactttgaaatgaggaaactttgataggatgtggattaagaactagggagggggaaagaaggatgggactattacatccacatgatacctctgatcaagtatttttgacatttactgtggataaattgtttttaagttttcatgaatgaattgctaagaaggggggaattcttttgctttttaccctcgactagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatcatttaaatgttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggatctcgagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 17 - DNA, 1611 bp, Artificial Sequence
  Synthetic construct - hIL-12 elasti (p40p35) ORF
atgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagactcgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 18 - DNA, 4846 bp, Artificial Sequence
  Synthetic construct - pORF-mIL-12 (p35p40)
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatgggtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccgttcctggagtaggggtacctggagtgggcggatctatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagtccccccgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctaggatgcaacggatgctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatcatttaaatgttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggatctcgagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 19 - DNA, 1623 bp, Artificial Sequence
  Synthetic construct - mIL-12 (p35p40) ORF
atgggtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccgttcctggagtaggggtacctggagtgggcggatctatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagtccccccgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctag

SEQ ID NO: 20 - DNA, 12718 bp, Artificial Sequence
  Synthetic construct - pHR′. cPPT.EF.CD19delta TmpkF105YR200A.WPRE.SIN
aattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggcatcctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttgccggcggggctgcagggatggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggcctacacaggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatcgccactgccacagagaagccgctgggggagctatggaagtgaggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattataatagatttagcaggaattgaactaggagtggagcacacaggcaaagctgcagaagtacttggaagaagccaccagagatactcacgattctgcacatacctggctaatcccagatcctaaggattacattaagtttactaacatttatataatgatttatagtttaaagtataaacttatctaatttactattctgacagatattaattaatcctcaaatatcataagagatgattactattatccccatttaacacaagaggaaactgagagggaaagatgttgaagtaattttcccacaattacagcatccgttagttacgactctatgatcttctgacacaaattccatttactcctcaccctatgactcagtcgaatatatcaaagttatggacattatgctaagtaacaaattacccttttatatagtaaatactgagtagattgagagaagaaattgtttgcaaacctgaatagcttcaagaagaagagaagtgaggataagaataacagttgtcatttaacaagttttaacaagtaacttggttagaaagggattcaaatgcataaagcaagggataaatttttctggcaacaagactatacaatataaccttaaatatgacttcaaataattgttggaacttgataaaactaattaaatattattgaagattatcaatattataaatgtaatttacttttaaaaagggaacatagaaatgtgtatcattagagtagaaaacaatccttattatcacaatttgtcaaaacaagtttgttattaacacaagtagaatactgcattcaattaagttgactgcagattttgtgttttgttaaaattagaaagagataacaacaatttgaattattgaaagtaacatgtaaatagttctacatacgttcttttgacatcttgttcaatcattgatcgaagttctttatcttggaagaatttgttccaaagactctgaaataaggaaaacaatctattatatagtctcacacctttgttttacttttagtgatttcaatttaataatgtaaatggttaaaatttattcttctctgagatcatttcacattgcagatagaaaacctgagactggggtaatttttattaaaatctaatttaatctcagaaacacatctttattctaacatcaatttttccagtttgatattatcatataaagtcagccttcctcatctgcaggttccacaacaaaaatccaaccaactgtggatcaaaaatattgggaaaaaattaaaaatagcaatacaacaataaaaaaatacaaatcagaaaaacagcacagtataacaactttatttagcatttacaatctattaggtattataagtaatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgcc

SEQ ID NO: 21 - DNA, 3513 bp, Artificial Sequence
  Synthetic construct - pHR Backbone
aattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgag

SEQ ID NO: 22 - DNA, 11122 bp, Artificial Sequence
  Synthetic construct - pHR Backbone
aattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattataatagatttagcaggaattgaactaggagtggagcacacaggcaaagctgcagaagtacttggaagaagccaccagagatactcacgattctgcacatacctggctaatcccagatcctaaggattacattaagtttactaacatttatataatgatttatagtttaaagtataaacttatctaatttactattctgacagatattaattaatcctcaaatatcataagagatgattactattatccccatttaacacaagaggaaactgagagggaaagatgttgaagtaattttcccacaattacagcatccgttagttacgactctatgatcttctgacacaaattccatttactcctcaccctatgactcagtcgaatatatcaaagttatggacattatgctaagtaacaaattacccttttatatagtaaatactgagtagattgagagaagaaattgtttgcaaacctgaatagcttcaagaagaagagaagtgaggataagaataacagttgtcatttaacaagttttaacaagtaacttggttagaaagggattcaaatgcataaagcaagggataaatttttctggcaacaagactatacaatataaccttaaatatgacttcaaataattgttggaacttgataaaactaattaaatattattgaagattatcaatattataaatgtaatttacttttaaaaagggaacatagaaatgtgtatcattagagtagaaaacaatccttattatcacaatttgtcaaaacaagtttgttattaacacaagtagaatactgcattcaattaagttgactgcagattttgtgttttgttaaaattagaaagagataacaacaatttgaattattgaaagtaacatgtaaatagttctacatacgttcttttgacatcttgttcaatcattgatcgaagttctttatcttggaagaatttgttccaaagactctgaaataaggaaaacaatctattatatagtctcacacctttgttttacttttagtgatttcaatttaataatgtaaatggttaaaatttattcttctctgagatcatttcacattgcagatagaaaacctgagactggggtaatttttattaaaatctaatttaatctcagaaacacatctttattctaacatcaatttttccagtttgatattatcatataaagtcagccttcctcatctgcaggttccacaacaaaaatccaaccaactgtggatcaaaaatattgggaaaaaattaaaaatagcaatacaacaataaaaaaatacaaatcagaaaaacagcacagtataacaactttatttagcatttacaatctattaggtattataagtaatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgcc

SEQ ID NO: 23 - protein, 8 aa, Artificial Sequence
  Synthetic construct
EAQNTTYL